(19) United States
(12) Patent Application Publication
(10) Pub. No.: US 2019/0345446 A1
(43) Pub. Date: Nov. 14, 2019

(54) COMPOSITIONS AND METHODS FOR MODULATING TH-17 AND TH-1 CELL BALANCE

(71) Applicants: The Broad Institute, Inc., Cambridge, MA (US); Massachusetts Institute of Technology, Cambridge, MA (US); The Brigham and Women's Hospital, Inc., Boston, MA (US)

(72) Inventors: Chao Wang, Boston, MA (US); Vijay K. Kuchroo, Boston, MA (US); Aviv Regev, Cambridge, MA (US); Gerd Meyer zu Hörste, Boston, MA (US)

(21) Appl. No.: 16/394,747

(22) Filed: Apr. 25, 2019

Related U.S. Application Data
(60) Provisional application No. 62/662,579, filed on Apr. 25, 2018.

(51) Int. Cl. C12N 5/0783; A61K 35/17; C12N 15/90; G01N 33/50; C12N 15/11; C12N 9/22; C07K 14/725

(52) U.S. Cl. CPC C12N 5/0636; A61K 35/17; C12N 15/907; G01N 33/505; C12N 2800/80; C12N 9/22; C07K 14/7051; C12N 2310/20; C12N 15/11

(57) ABSTRACT

The subject matter disclosed herein is generally directed to compositions and methods related to FAS-STAT1 interactions. Modulation of FAS-STAT1 interaction can be used to shift Th17-to-Th1 cell balance. The methods and cell compositions can be used for treating autoimmunity in a subject in need thereof. Cell compositions with altered FAS-STAT1 interactions can be used for adoptive cell transfer. The invention also relates to screening for agents capable of modulating FAS-STAT1 interactions.

### CROSS-REFERENCE TO RELATED APPLICATIONS

This application claims the benefit of U.S. Provisional Application No. 62/662,579, filed Apr. 25, 2018. The entire contents of the above-identified application are hereby fully incorporated herein by reference.

### STATEMENT REGARDING FEDERALLY SPONSORED RESEARCH

This invention was made with government support under Grant No.(s) NS30843, A10562999, NS076410, A1039671, AI045757, A1073748 awarded by the National Institutes of Health. The government has certain rights in the invention.

### REFERENCE TO AN ELECTRONIC SEQUENCE LISTING

The contents of the electronic sequence listing (BROD_2510US_ST25.txt”; Size is 10 Kilobytes and it was created on Mar. 19, 2019) is herein incorporated by reference in its entirety.

### TECHNICAL FIELD

The subject matter disclosed herein is generally directed to compositions and methods related to FAS-STAT1 interactions.

### BACKGROUND

Interleukin (IL)-17-producing helper T cells (Th17 cells) have been identified as a distinct lineage of CD4⁺ T helper cells producing IL-17A and IL-17F and are critical drivers of autoimmune tissue inflammation in experimental autoimmune encephalomyelitis (EAE) and in other autoimmune conditions (Korn et al., 2009). In a recent study, Applicants have shown that the Th17 cell differentiation program is regulated through two self-reinforcing and mutually antagonistic modules of positive and negative regulators (Yosef et al., 2013). This model was supported by transcriptional silencing and genetic ablation experiments (Yosef et al., 2013), as well as by chromatin immunoprecipitation (ChIP)-seq data (Xiao et al., 2014). The positive regulators promote the Th17 cell program while inhibiting the transcriptional programs of other T helper (Th) cell lineages. This suggests that there is not only a need for positive regulators to push the differentiation into a positive direction but also for concurrent inhibition of opposing differentiation programs to achieve unidirectional T cell differentiation. Other studies also support such a mutually antagonistic design in other Th lineages (O'Shea and Paul, 2010), however, how this is achieved for Th17 cells has not been elucidated.

One of the key positive regulators identified by this model was the prototypic death receptor Fas (Yosef et al., 2013). The network model predicted that while promoting Th17 cell differentiation, Fas would also negatively impact the differentiation of other T cell subsets. Initial analysis of in vitro derived Th17 cells from Fas deficient mice showed the predicted reduction in Th17 cell related gene expression but the underlying mechanism has not been identified.

Fas has been extensively studied as a trigger of apoptosis in various cell types (Strasser et al., 2009). Indeed, mice carrying a spontaneous mutation of Fas (the lymphoproliferation (lpr) strain (Watanabe-Fukunaga et al., 1992)) or FasL (Takahashi et al., 1994) develop progressive lymphadenopathy and splenomegaly with accumulation of lymphocytes with an atypical TCRαβ⁺CD4⁻CD8⁻B220⁺ phenotype due to a lack of apoptosis of activated T cells (Fortner and Budd, 2005). In addition, Fas signalling has other, non-apoptotic, functions including costimulatory and pro-proliferative effects (Kennedy et al., 1999; Paulsen et al., 2011). T cells deficient in components of the Fas signalling pathway such as FADD (Walsh et al., 1998) or caspase-8 (Kennedy et al., 1999) have impaired T cell activation and proliferation. Fas activation may thus have opposing outcomes that strongly depend on the cellular context, but whether such non-apoptotic Fas signals also control the differentiation of T cells and specifically of Th17 cells has not been elucidated.

In particular, Fas has a suppressive effect on spontaneous systemic autoimmunity, but a seemingly paradoxical promoting effect on induced autoimmunity. Aging lpr mice spontaneously develop disease signs resembling human systemic lupus erythematosus (SLE)-like disease (Cohen and Eisenberg, 1991) and are therefore widely used as an SLE-animal model. Similarly, human patients with loss-of-function or dominant-negative mutations of Fas accumulate lymphocytes and develop various autoimmune phenomena (Holzelova et al., 2004). Fas thus suppresses spontaneous systemic autoimmunity but promotes induced autoimmunity since Fas-deficient 1pr mice are strongly protected from EAE (Sabelko et al., 1997; Waldner et al., 1997). The mechanisms underlying these enigmatic observations have not been elucidated.

### SUMMARY

In certain example embodiments, provided are compositions and methods based on the discovery that FAS-STAT1 interactions control T cell balance. The death receptor Fas removes activated lymphocytes through apoptosis. Previous transcriptional profiling predicted that Fas positively regulates interleukin (IL)-17-producing T helper (Th17) cells. Applicants hypothesized that their model of opposing and mutually antagonistic gene regulation may provide a starting point to identify the mechanism to explain these seemingly paradoxical observations. Applicants here demonstrate that Fas promoted the generation and stability of Th17 cells and prevented their differentiation into Th1 cells. Mice with T cell- and Th17 cell-specific deletion of Fas were protected from induced autoimmunity and Th17 cell differentiation and stability was impaired. Fas deficient Th17 cells instead developed a Th1 cell-like transcriptional profile, which Applicants predicted by a new algorithm to depend on STAT1. Experimentally, Fas indeed bound and sequestered STAT1 and Fas deficiency enhanced IL-6 induced STAT1 activation and nuclear translocation, whereas Fas-STAT1 double-deficiency reversed the transcriptional changes induced by Fas deficiency. Thus, the computational and experimental approach identified Fas as a regulator of the Th17-to-Th1 cell balance by controlling the availability of the opposing STAT1 and STAT3 proteins with direct impact on autoimmunity.

In one aspect, the present invention provides for an isolated T cell modified to comprise altered FAS-STAT1 binding.

In certain embodiments, the T cell is modified to express a recombinant polypeptide capable of antagonizing FAS-STAT1 interaction. In certain embodiments, the polypeptide does not affect the binding of FAS to FAS-L. In certain embodiments, the polypeptide does not affect the binding of FAS to FADD.

In certain embodiments, the T cell is modified to express a recombinant polypeptide that is capable of adopting a FAS ligand bound conformation, is inactivated for apoptotic signaling, and is able to bind to STAT1. Thus, the recombinant polypeptide is only able to antagonize FAS-STAT1 binding. In certain embodiments, the polypeptide does not affect the binding of FAS to FAS-L. In certain embodiments, the polypeptide does not affect the binding of FAS to FADD.

In certain embodiments, the T cell is modified to over-express STAT1. Thus, in certain embodiments, increased expression of STAT1 can saturate binding to FAS and shift T cell balance towards a Th1 phenotype.

In certain embodiments, the T cell is modified to abolish or knockdown expression or activity of STAT1 and is differentiated under Th17 conditions. The Th17 conditions may comprise cultures supplemented with IL-6 and TGF-β1 or supplemented with IL-1β, IL-6 and IL-23. The T cell may comprise a genetic modifying agent targeting STAT1. The genetic modifying agent may comprise a CRISPR system, a zinc finger nuclease system, a TALEN, or a meganuclease. The CRISPR system may comprise Cas9 or Cpf1 and target the STAT1 gene. The CRISPR system may comprise a Cas13 system and target STAT1 mRNA. The Cas13 system may comprise Cas13-ADAR.

In certain embodiments, the T cell is modified to comprise a non-silent mutation in FAS and/or STAT1, wherein the mutation inhibits FAS-STAT1 binding. The mutation may alter a post-translational modification site in FAS and/or STAT1 that alters FAS-STAT1 binding. The mutation may not inhibit FAS apoptotic signaling. The T cell may comprise a genetic modifying agent targeting FAS and/or STAT1. The genetic modifying agent may comprise a CRISPR system, a zinc finger nuclease system, a TALEN, or a meganuclease. The CRISPR system may comprise a Cas13 system and target FAS and/or STAT1 mRNA. The Cas13 system may comprise Cas13-ADAR.

In certain embodiments, the T cell is modified to decrease, but not eliminate expression or activity of FAS. The T cell may be differentiated under Th17 conditions. The Th17 conditions may comprise cultures supplemented with IL-6 and TGF-β1 or supplemented with IL-1β, IL-6 and IL-23. The T cell may comprise a genetic modifying agent targeting FAS. The genetic modifying agent may comprise a CRISPR system, a zinc finger nuclease system, a TALEN, or a meganuclease. The CRISPR system may comprise a Cas13 system and target FAS mRNA. The Cas13 system may comprise Cas13-ADAR.

In certain embodiments, the isolated T cell of any embodiment is a Th17 cell. In certain embodiments, the T cell is a naïve Th0 cell. In certain embodiments, the T cell is a tumor infiltrating lymphocyte (TIL). In certain embodiments, the T cell expresses an endogenous T cell receptor (TCR) or chimeric antigen receptor (CAR) specific for a tumor antigen. In certain embodiments, the T cell is expanded. In certain embodiments, the T cell is modified to express a suicide gene, wherein the modified T cell can be eliminated upon administration of a drug.

In another aspect, the present invention provides for a pharmaceutical composition comprising the isolated T cell of any of claims 2 to 4.

In another aspect, the present invention provides for a pharmaceutical composition comprising the isolated T cell of claim 8.

In another aspect, the present invention provides for a pharmaceutical composition comprising the isolated T cell of any of claims 16 to 22.

In another aspect, the present invention provides for a pharmaceutical composition comprising the isolated T cell of any of claims 23 to 29.

In another aspect, the present invention provides for a pharmaceutical composition comprising the isolated T cell of any of claims 5 to 7.

In another aspect, the present invention provides for a pharmaceutical composition comprising the isolated T cell of any of claims 9 to 15.

In another aspect, the present invention provides for a method of treating cancer comprising administering the pharmaceutical composition of claim 40 or 41 to a subject in need thereof, whereby a Th17 response is enhanced.

In another aspect, the present invention provides for a method of treating cancer comprising administering the pharmaceutical composition of any of claims 36 to 39 to a subject in need thereof, whereby a Th1 response is enhanced.

In another aspect, the present invention provides for a method of treating an inflammatory or autoimmune disease comprising administering the pharmaceutical composition of any of claims 36 to 39 to a subject in need thereof.

In another aspect, the present invention provides for a method of modulating T cell balance, the method comprising perturbing FAS-STAT1 binding in a T cell or a population of T cells. In certain embodiments, perturbing comprises introducing a genetic modifying agent targeting FAS and/or STAT1 to the T cell or population of T cells. The genetic modifying agent may comprise a CRISPR system, a zinc finger nuclease system, a TALEN, or a meganuclease. The CRISPR system may comprise a Cas13 system and target FAS and/or STAT1 mRNA. The Cas13 system may comprise Cas13-ADAR.

In certain embodiments, the T cell or population of T cells may be modified to comprise a non-silent mutation in FAS and/or STAT1, wherein the mutation inhibits FAS-STAT1 binding. The mutation may alter a post-translational modification site in FAS and/or STAT1. In certain embodiments, T cell differentiation is shifted towards Th1 cells and/or is shifted away from Th17 cells.

In certain embodiments, the T cell or population of T cells is modified to comprise a decrease or knockout in expression of STAT1. In certain embodiments, T cell differentiation is shifted towards Th17 cells and/or is shifted away from Th1 cells.

In certain embodiments, the T cell or population of T cells is modified to comprise a decrease in expression of FAS. In certain embodiments, FAS mRNA is targeted and the decrease is temporary. In certain embodiments, T cell differentiation is shifted towards Th1 cells and/or is shifted away from Th17 cells.

In certain embodiments, the CRISPR system is administered as a ribonucleoprotein (RNP) complex.

In certain embodiments, modulating T cell balance comprises contacting the T cell or population of T cells with an inhibitor of FAS-STAT1 binding. In certain embodiments, modulating T cell balance comprises increasing expression of STAT1 in the T cell or population of T cells. In certain embodiments, T cell differentiation is shifted towards Th1 cells and/or is shifted away from Th17 cells.

In certain embodiments, the T cell or population of T cells comprise naïve Th0 T cells. The cells may be cultured under Th1 or Th17 conditions. The Th17 conditions may comprise cultures supplemented with IL-6 and TGF-β1 or supplemented with IL-1β, IL-6 and IL-23. In certain embodiments, FAS is bound by FAS ligand.

In another aspect, the present invention provides for a method of modulating an immune response in a subject in need thereof, the method comprising administering to the subject a therapeutically effective amount of an inhibitor of FAS-STAT1 binding. The method may be for treating an aberrant immune response in said subject. The method may be for treating an autoimmune disease. The autoimmune disease may be selected from Multiple Sclerosis (MS), Irritable Bowel Disease (IBD), Crohn's disease, spondyloarthritides, Systemic Lupus Erythematosus (SLE), Vitiligo, rheumatoid arthritis, psoriasis, Sjögren's syndrome, and diabetes. The method may be for treating an inflammatory disorder. The inflammatory disorder may be selected from psoriasis, inflammatory bowel diseases (IBD), allergic asthma, food allergies and rheumatoid arthritis.

In certain embodiments, the inhibitor of FAS-STAT1 binding does not affect the binding of FAS to FAS-L. In certain embodiments, the inhibitor of FAS-STAT1 binding does not affect the binding of FAS to FADD. In certain embodiments, the inhibitor binds to the cytoplasmic domain of FAS. In certain embodiments, the inhibitor does not bind to the extracellular domain of FAS.

In certain embodiments, the inhibitor is an antibody, antibody fragment, intrabody, antibody-like protein scaffold, polypeptide, genetic modifying agent, or small molecule. The genetic modifying agent may comprise a CRISPR system, a zinc finger nuclease system, a TALEN, or a meganuclease.

In another aspect, the present invention provides for a pharmaceutical composition comprising an inhibitor of FAS-STAT1 binding. The inhibitor of FAS-STAT1 binding may not affect the binding of FAS to FAS-L. The inhibitor of FAS-STAT1 binding may not affect the binding of FAS to FADD. The inhibitor may bind to the cytoplasmic domain of FAS. The inhibitor may not bind to the extracellular domain of FAS. In certain embodiments, the inhibitor is an antibody, antibody fragment, intrabody, antibody-like protein scaffold, polypeptide, genetic modifying agent, or small molecule.

In certain embodiments, modulating T cell balance comprises providing the T cell or population of T cells with a FAS polypeptide, wherein said polypeptide is able to bind to STAT1. The polypeptide may adopt a FAS ligand bound conformation and may be inactivated for apoptotic signaling. In certain embodiments, T cell differentiation is shifted towards Th17 cells and/or is shifted away from Th1 cells. In certain embodiments, providing a FAS polypeptide comprises providing a nucleic acid encoding the polypeptide. The nucleic acid may be provided as a vector. The polypeptide may be a membrane bound polypeptide. The polypeptide may not bind to FAS-L. The polypeptide may not comprise the extracellular domain of FAS. Binding of the polypeptide to STAT1 may not lead to phosphorylation of STAT1. Binding of the polypeptide to STAT1 may prevent or reduce nuclear translocation of STAT1.

In another aspect, the present invention provides for a method of treating cancer or an infectious disease in a subject in need thereof comprising: isolating Th17 cells from the blood of the subject; transforming the isolated Th17 cells with one or more vectors encoding: (i) a CAR or endogenous TCR directed against a tumor antigen or an infectious disease antigen, and (ii) a CRISPR system targeting STAT1; and administering the transformed Th17 cells to the subject.

In another aspect, the present invention provides for a method of treating autoimmunity in a subject in need thereof comprising: isolating Th17 cells from the blood of the subject; transforming the isolated Th17 cells with one or more vectors encoding a CRISPR system targeting FAS; and administering the Th17 FAS mutant cells to the subject.

In another aspect, the present invention provides for a method of screening for agents capable of modulating FAS-STAT1 interaction comprising: differentiating naïve Th0 T cells under Th17 conditions in the presence of one or more agents that specifically bind to FAS and/or STAT1; and detecting one or more Th17 or Th1 markers, wherein decreased Th17 markers or increased Th1 markers indicates an agent that modulates the interaction. In certain embodiments, the cells are differentiated under pathogenic Th17 conditions. In certain embodiments, the one or more Th17 markers comprises IL-17A.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1G—Fas promotes Th17 cell responses and represses Th1 cell differentiation. (FIG. 1A) Th17 cell transcriptional model adapted from (Yosef et al., 2013), consists of two self reinforcing and mutually antagonistic modules of positive (red nodes) and negative (blue nodes) regulators with opposite effects on Th17 cell signature genes (grey nodes, bottom) and signature genes of other CD4⁺ T cells (grey nodes, top). A blue edge from node A to B indicates that silencing of A downregulates B; a red edge indicates that silencing of A upregulates B. (FIG. 1B) Naïve CD4⁺CD62L^{high}CD44^{low}CD25⁻ T cells were sorted from Fas^{fl/fl} mice (WT) and Cd4^{cre}Fas^{fl/fl} mice, differentiated with TGF-β1, IL-6 or IL-1β, IL-6, IL-23 or IL-12 and analyzed by intracellular cytokine staining after 4 days. (FIG. 1C) Supernatants of cultures described in B were analyzed by ELISA. One out of 5 independent experiments is shown in A and B. (FIG. 1D) Cytokine positive cells averaged between technical replicates in individual experiments were quantified. (FIG. 1E) EAE was induced in WT (n=15) and Cd4^{cre}Fas^{fl/fl} (n=12) mice by subcutaneous immunization with 100 μg of MOG₃₅₋₅₅ peptide. Mice were monitored daily for EAE signs. (FIG. 1F) At day 28 after immunization, inflammatory lesions in the CNS parenchyma and in the meninges were quantified histologically. Data in E, F are summed from three independent experiments. (FIG. 1G) CNS infiltrating lymphocytes were extracted at peak of EAE and stained for intracellular cytokines. One out of six independent experiments is shown in G. *p<0.05, **p<0.01. See also FIG. 51.

FIGS. 2A-2F—Fas is required in Th17 cells to promote their differentiation and in vivo function. (FIG. 2A) Naïve CD4⁺CD62L^{high}CD44^{low}CD25⁻ T cells were sorted from wildtype Il17a^{cre}Fas^{fl/wt} (WT) and Il17a^{cre}Fas^{fl/fl} mice, differentiated with TGF-β1, IL-6 or IL-1β, IL-6, IL-23 or IL-12 and analyzed for intracellular cytokines after 4 days. (FIG. 2B) Supernatants of cell cultures described in A were analyzed by ELISA. (FIG. 2C) Cytokine positive cells were averaged between technical replicates. (FIG. 2D) EAE was induced in WT (n=28) and Il17a^{cre}Fas^{fl/fl} (n=15) mice by subcutaneous immunization with 100 μg of MOG₃₅₋₅₅ peptide. Mice were monitored daily for clinical EAE signs. (FIG. 2E) At day 28 after immunization, inflammatory lesions in the CNS parenchyma and in the meninges was quantified histologically. Data in D and E are summed from three experiments. (FIG. 2F) CNS infiltrating lymphocytes were extracted at peak of EAE and stained for intracellular cytokines. One out of four experiments is shown in A and B and one out of five experiments is shown in F. *p<0.05, **p<0.01, NS not significant, ND not detected. See also Figure S2.

FIGS. 3A-3K—Fas promotes the encephalitogenicity and stability of Th17 cells. (FIG. 3A) Naïve CD4⁺CD62L^{high}CD44^{low}CD25⁻ T cells were sorted from wildtype (WT) 2D2 and Fas^{-/-}2D2 mice and differentiated with TGF-β1, IL-6 and IL-23 (Methods). After 7 days, T cells were intravenously injected into C57BL/6 WT recipients. EAE score was assessed daily. One out of 3 independent experiments is shown. (FIG. 3B) At day 28 after transfer, inflammatory lesions in the CNS parenchyma and in the meninges were quantified histologically. (FIG. 3C) CNS infiltrating lymphocytes from score matched mice at peak of EAE were stained for intracellular cytokines. Gating was on live CD4+Vα3.2⁺Vβ11⁺ cells. (FIG. 3D) CNS-infiltrating leukocytes and the proportion of cytokine producing CD4+Vα3.2⁺Vβ11⁺ cells were quantified. Data in B, D are merged from 3 independent experiments. (FIG. 3E) Th17 cells were differentiated in vitro from CD45.1⁺ WT-2D2 and CD45.2⁺ Fas^{-/-}2D2 mice, mixed at equal ratios and injected intravenously into wildtype CD90.1 recipients. (FIG. 3F) The relative proportion of CD45.1⁺ and CD90.1⁺ CD4⁺ T cells was analyzed by flow cytometry before transfer and after transfer at peak of EAE. (G) At peak of EAE, CNS infiltrating leukocytes were stained for intracellular cytokines. Gating was on either CD4⁺CD45.1⁻CD90.1⁺ host cells (left), CD4⁺CD45.1⁺CD90.1⁻ WT donor cells (middle), and CD4⁺CD45.1⁻CD90.1⁻ Fas^{-/-} donor cells (right). (FIG. 3H) EAE was induced by immunization with 100 μg of MOG₃₅₋₅₅ peptide in Il17a^{cre}R26^{RFP} (squares, n=5) and Il17a^{cre}R26^{RFP}Fas^{-/-} (triangles, n=4) mice and the proportion of RFP⁺CD4⁺ was analyzed by flow cytometry in the peripheral blood. (FIG. 3I) At peak of EAE, leukocytes were extracted from inguinal lymph nodes (iLN), spleen and the CNS of score-matched mice and analyzed for intracellular cytokine production. (FIG. 3J) The proportion of RFP⁺CD4⁺ cells was analyzed by flow cytometry in the peripheral blood as in A in Il17a^{cre}R26^{RFP}Fas^{fl/wt} (squares, n=11) and Il17a^{cre}R26^{RFP}Fas^{fl/fl} (inverted triangles, n=7) mice. (FIG. 3K) Leukocytes from iLN, spleen and the CNS of Il17a^{cre}R26^{RFP}Fas^{fl/wt} and Il17a^{cre}R26^{RFP}Fas^{fl/fl} mice were analyzed by intracellular cytokine staining at the peak of EAE as in I. Data in G, I are merged from 2 and 3 independent experiments, respectively. One of 3 independent experiments is shown in E-G, I, K. See also Figure S3.

FIGS. 4A-4F—Fas deficient Th17 cells exhibit a Th1 cell-like expression profile that is predicted to be partially controlled by STAT1. (FIG. 4A) Naïve CD4⁺CD62L^{high}CD44^{low}CD25⁻ T cells from wildtype (WT) and Fas^{-/-} mice, were differentiated in the presence of TGF-β1, IL-6 (Th17) or no cytokines (Th0) for 48 hours. Total RNA from the cells was analyzed by RNA-sequencing (Methods). A heatmap of fold change (log base 2) for differentially expressed genes (FDR<0.05 in Th17 or Th0) is shown. (FIG. 4B) Gene set enrichment analysis (GSEA) identified most significant enrichment of the interferon-γ signalling pathway. Genes ranked by differential expression (Fas^{-/-} vs WT) in Th17 cell conditions were queried against Reactome pathway datasets in MSigDB (Methods). (FIG. 4C) The expression of selected transcripts was quantified in an independent set of Th17 cell samples (n=5 per genotype) differentiated using TGF-β1, IL-6 from naïve CD4⁺ T cells for 72 hours by quantitative real-time PCR. ND not detected. NS not significant. (FIG. 4D) A network algorithm that considered evidence from protein-protein interaction databases (PPI), the RNA-seq expression data, and transcription factor motif enrichment was used to rank TFs (yellow) that were most likely influenced by Fas (purple). Intermediate nodes (grey) had to be expressed in Th17 or Th0 conditions (FPKM>3). Arrows indicate directed interactions, such as phosphorylation. Stat1 received most evidence (green) among network paths containing at most one intermediate node (Methods). (FIG. 4E) Close up of the PPI network indicating paths from Fas (purple) to Stat1 (green). (FIG. 4F) Naïve T cells were differentiated with TGF-β1 and IL-6 for 72 hours and RNA was collected at several time points. The expression of Stat1 and Irf1 was analyzed by qPCR. The first time point is derived from naïve, pre-differentiated cells (i.e. 0 hours) despite the logarithmic scale. See also Figure S4.

FIGS. 5A-5E—Fas interacts with STAT1 and inhibits STAT1 phosphorylation and nuclear translocation. (FIG. 5A) Sorted naïve CD4⁺CD62L^{high}CD44^{low}CD25⁻ T cells from wildtype (WT) and Fas^{-/-} mice (5*10⁵ per timepoint) were stimulated with IL-6 as indicated. Cells were lysed and analyzed by Western blotting using the indicated antibodies (Ab). Phospho-STAT1 (S727) was visualized first; the membrane was then stripped, and re-probed with anti-STAT1, and anti-βActin. (FIG. 5B) Sorted naïve CD4⁺ T cells (5*10⁵ per timepoint) from WT and Fas^{-/-} mice were stimulated as in A. Nuclear fractions were purified (Methods) and analyzed as in A. (FIG. 5C) Sorted naïve CD4⁺ T cells from WT and Fas^{-/-} mice were incubated with IL-6 for 30 minutes and stained for phospho-STAT1 (S727). Mean fluorescence of phospho-STAT1 staining per cell (left) and per nucleus (right) was quantified by confocal microscopy in >50 randomly chosen cells. (FIG. 5D) EL4 cells (1*10⁸/lane) were stimulated with biotinylated hamster IgG (DISCiso) or biotinylated anti-Fas Ab (DISC+) and streptavidin for 15 minutes or left untreated on ice for 15 minutes (DISC−). Anti-Fas Ab was then added to the DISC-sample. Co-immunoprecipitation (Co-IP) was then performed using Protein G magnetic beads. Input and elute fractions from the Co-IP were analyzed using the indicated Ab. (FIG. 5E) Primary CD4⁺ T cells were MACS purified (5*10⁷/lane) and either left unstimulated on ice for 15 minutes (DISC−) or stimulated with recombinant multimerized FasL (DISC+). Anti-Fas Ab or armenian hamster IgG was then added to the lysate and co-IP was performed using Protein G magnetic beads. Western blotting was performed using the indicated antibodies. One of 3 independent experiments is depicted in A, B, C, and E. One of 6 independent experiments is depicted in D, min minutes, exp exposure. See also Figure S5.

FIGS. 6A-6F—STAT1 deficiency rescues the Th17 cell defect of Fas deficient CD4⁺ T cells. (FIG. 6A) Naïve CD4⁺CD62L^{high}CD44^{low}CD25⁻ T cells were sorted from wildtype (WT), Stat1 deficient (Stat1^{-/-}), Fas^{-/-}, or Stat1^{-/-}Fas^{-/-} mice and differentiated with TGF-β1, IL-6 or IL-1β, IL-6, IL-23 or IL-12 for 4 days and analyzed by intracellular cytokine staining. (FIG. 6B) The concentrations of IL-17A and IFN-γ in supernatants of cultures described in A were analyzed by ELISA. *p<0.05, **p<0.01, NS not significant. (FIG. 6C) Sorted naïve CD4⁺ T cells were differentiated in the presence of TGF-β1, IL-6 for 72 hours and subjected to nanostring analysis. Transcripts with >1.25 fold regulation in Fas^{-/-} vs. WT conditions were sorted based on the Fas^{-/-} vs. WT fold-regulation (dark blue). The Stat1^{-/-}Fas^{-/-} vs. WT fold-regulation was then plotted for these transcripts (light blue). (FIG. 6D) The fold expression change in nanostring in Fas^{-/-} versus WT samples (x-axis) was plotted against Stat1^{-/-}Fas^{-/-} vs. Fas^{-/-} samples (y-axis) on a log₂ scale. Transcripts differentially expressed in the RNA-seq dataset (FIGS. 3A-3K) are highlighted in red. Data in C-D are merged from three independent experiments with one biological replicate in each experiment. (FIG. 6E) 4*10⁶ MACS purified CD4⁺ T cells from WT, Stat1^{-/-}, Fas^{-/-}, or Stat1^{-/-}Fas^{-/-} donors were intravenously injected into Rag1^{-/-} mice (n=5 per group) and 10-12 days later recipients were immunized with 100 μg MOG₃₅₋₅₅ peptide. Mice were monitored daily for clinical EAE signs. (FIG. 6F) At day 35 after immunization, inflammatory lesions in the CNS parenchyma and in the meninges were analyzed histologically. One representative of three independent experiments is shown in E and F. See also Figure S6.

FIGS. 7A-7G—Fas^{-/-}CD4⁺ T cells show impaired Th17 and Th2 cell responses. (FIG. 7A) Naïve CD4⁺CD62L^{high}CD44^{low}CD25⁻ T cells were sorted from peripheral lymphoid organs of wildtype (WT) and Fas^{-/-} mice, differentiated in the presence of TGF-β1, IL-6 or IL-1β, IL-6, IL-23 or IL-12 for 4 days and analyzed by intracellular cytokine staining. (FIG. 7B) The concentrations of IL-17A and IFN-γ in the cell culture supernatants of T cell cultures described in A were analyzed by ELISA. One representative out of 12 independent experiments is depicted in A and B. (FIG. 7C) Sorted naïve CD4⁺ T cells from WT and Fas^{-/-} mice were differentiated as in A in the presence of anti-IFN-γ (clone XMG1.2) or rat IgG1κ isotype control at 5 μg/ml and analyzed by intracellular cytokine staining. (FIG. 7D) Sorted naïve CD4⁺ T cells from WT and Fas^{-/-} mice were cultured in the presence of IL-4 for 4 days and analyzed by intracellular cytokine staining for the proportion of IL-5⁺ and IL-13⁺ cells. (FIG. 7E) Sorted naïve CD4⁺ T cells from WT and Fas^{-/-} mice were cultured in the presence of TGF-β1 and the proportion of FoxP3⁺CD4⁺ cells was quantified by intracellular staining. One representative out of three independent experiments is depicted in C-E. (FIG. 7F) Naïve CD4⁺ T cells from WT mice were cultured in the presence of TGF-β1 and RNA was extracted and expression of Fas was quantified by qPCR at various time-points as in B. (FIG. 7G) Cells cultured as in F were analyzed for Fas surface expression by flow cytometry. Please note that the scaling of the axes is identical between panels S1F and S3A and between S1G and S3B, respectively. NS not significant, *p<0.05, **p<0.01.

FIGS. 8A-8G—In vivo Th17 cell responses are impaired in the absence of Fas. (FIG. 8A) Active EAE was induced in wildtype (n=19) and Fas^{-/-} (n=15) mice by subcutaneous immunization with 100 μg of MOG₃₅₋₅₅ peptide emulsified in complete Freund's adjuvant (CFA) together with intraperitoneal injection of pertussis toxin on day 0 and 2. Mice were monitored daily for clinical EAE signs. Data are summed from three independent experiments. (FIG. 8B) CNS infiltrating lymphocytes were extracted from score matched mice with EAE and stained for intracellular cytokines at peak of EAE. (FIG. 8C) WT and Fas^{-/-} mice were immunized with 100 μg MOG₃₅₋₅₅ in CFA without pertussis toxin. After 7 days draining lymph node cells were restimulated with increasing amounts of MOG₃₅₋₅₅ together with IL-23. After 5 days in culture, cytokine production in CD4⁺ cells was quantified by intracellular cytokine staining. One out of three experiments is shown. (FIG. 8D) At peak of EAE, CNS infiltrating leukocytes were extracted from WT and Cd4^{cre}Fas^{fl/fl} mice, counted manually, stained for intracellular cytokines, and the proportion of cytokine producing cells of CD4⁺ cells was quantified. (FIG. 8E) At peak of EAE, CNS infiltrating leukocytes were extracted from wildtype Il17a^{cre}Fas^{fl/wt} (WT) and Il17a^{cre}Fas^{fl/fl} mice, counted manually, stained for intracellular cytokines, and the proportion of cytokine producing cells of CD4⁺ cells was quantified. Data in D and E are each summed from three independent experiments. (FIG. 8F) EAE was induced by immunization with 100 μg of MOG₃₅₋₅₅ peptide in complete Freund's adjuvant and intraperitoneal injection of pertussis on day 0 and 2 toxin in Il17a^{cre}R26^{RFP} and Il17a^{cre}R26^{RFP}Fas^{-/-} mice. At the peak of EAE, leukocytes were extracted from inguinal lymph nodes (iLN), spleen and the CNS of score-matched mice and analyzed by intracellular cytokine staining. (FIG. 8G) EAE was induced as in F and at the peak of EAE leukocytes from the iLN, spleen, and the CNS of score-matched Il17a^{cre}R26^{RFP}Fas^{fl/wt} and Il17a^{cre}R26^{RFP}Fas^{fl/fl} mice were analyzed by intracellular cytokine staining. One representative of 3 independent experiments is depicted in F-G. NS not significant, *p<0.05.

FIGS. 9A-9J—Fas is expressed by all T helper cell lineages and Caspase-inhibition or -deficiency do not replicate the Fas^{-/-} phenotype. (FIG. 9A) Naïve CD4⁺CD62L^{high}CD44^{low}CD25⁻ T cells were sorted from WT mice, differentiated in the presence of the indicated cytokines and RNA was extracted before and after 1, 2, 4, 6, 8, 12, 16, 24, 36, 48, 72, 96 hours of differentiation and analyzed for the expression of Fas by qPCR using Gapdh as housekeeping gene. (FIG. 9B) Sorted naïve WT CD4⁺ T cells were cultured as in A and the proportion of Fas⁺CD4⁺ live T cells was quantified by surface staining and flow cytometry before and after 24, 48, 96 hours of culturing. (C, D) Sorted naïve WT CD4⁺ T cells were cultured in the presence of different combinations of IL-12, TGF-β1, IL-1β, IL-6, IL-23, and TNFα. Fas expression was quantified after 96 hours by qPCR (FIG. 9C) and the proportion of Fas⁺CD4⁺ live T cells was quantified by flow cyometry (FIG. 9D). (FIG. 9E) Naïve CD4⁺CD62L^{high}CD44^{low}CD25⁻ T cells were sorted from wildtype (WT) mice, cultured in the presence of TGF-β1, IL-6 or IL-1β, IL-6, IL-23 or IL-12 for 4 days and analyzed by staining for Annexin V⁺ and 7AAD. (FIG. 9F) The proportion of Annexin V⁺ cells was quantified as in E. (FIG. 9G) Naïve T cell cultures described in A were TUNEL stained after 96 hours of differentiation. One representative out of 4 independent experiments is depicted in A-C. (FIG. 9H) Naïve CD4⁺ T cells were sorted from WT mice and cultured in the presence of TGF-β1, IL-6 or IL-1β, IL-6, IL-23 or IL-12 for 4 days and in the presence of DMSO vehicle only (top row), Caspase 1 inhibitor (C1 Inh, Z-YVAD-FMK, second row), Caspase 3 inhibitor (C3 Inh, Z-DEVD-FMK, third row), or Caspase 8 inhibitor (C8 Inh, Z-IETD-FMK, fourth row). The cells were analyzed by intracellular cytokine staining and flow cytometry. (FIG. 9I) Supernatants from T cell cultures described in H were analyzed by ELISA. (FIG. 9J) Naïve CD4⁺ T cells were sorted from WT, Fas^{-/-}, Casp3^{-/-}, and Fas^{-/-}Casp3^{-/-} mice and differentiated in the presence of TGF-β1, IL-6 or IL-1β, IL-6, IL-23 or IL-12 for 4 days and intracellular cytokine staining was performed. One representative out of three independent experiments is shown in C and K. NS not significant, *p<0.05, **p<0.01.

FIGS. 10A-10B—Expression of STAT-family proteins in Fas-deficient Th17 cells. (FIG. 10A) Fragments Per Kilobase of transcript per Million mapped reads (FPKM) values of STAT-protein family transcripts were extracted from the RNA-sequencing dataset depicted in FIG. 4 (n=3 per genotype) and compared between WT and Fas^{-/-} samples. The Student's t-test for unrelated samples was used to test for significant differences. Please note that differences between Stat1 FPKM values did not reach the significance threshold when adjusting for multiple hypothesis testing (q-value (i.e. adjusted p-value)>0.05), but did reach the significance threshold when using Student's t-test. (FIG. 10B) Naïve CD4⁺CD62L^{high}CD44^{low}CD25⁻ T cells were sorted from wildtype (WT) and Fas^{-/-} mice and cultured in the presence of TGF-β1, IL-6 for 72 hours. The expression of genes encoding members of the STAT-protein family was quantified by quantitative real-time PCR using Gapdh as housekeeping gene. *p<0.05, NS not significant.

FIGS. 11A-11G—Fas inversely regulates the IL-6 induced activation of STAT1 and STAT3. (FIG. 11A) Sorted naïve CD4⁺CD62L^{high}CD44^{low}CD25⁻ T cells from wildtype (WT) and Fas^{-/-} mice (5*10⁵ per timepoint) were stimulated with IFN-γ for the indicated times. Cells were then lysed and analyzed by Western blotting using the indicated antibodies. Phospho-(p)STAT1 (S727) was visualized first; the membrane was then stripped, and reprobed with anti-STAT1, and anti-βActin. (FIG. 11B) Sorted naïve CD4⁺ T cells from WT and Fas^{-/-} mice were stimulated with IL-6 for different durations, fixed and stained for intracellular pSTAT1 (S727) and pSTAT3 (Y705). The proportion of pSTAT1⁺ and pSTAT3⁺ cells was quantified. (FIG. 11C) Sorted naïve CD4⁺ T cells from WT and Fas^{-/-} mice were incubated with IL-6 for 30 minutes, stained for pSTAT1 (S727) and pSTAT3 (Y705), and analyzed by confocal microscopy. Scale bar represents 5 μm. (FIG. 11D) The mean fluorescence of pSTAT1 (S727) staining as shown in C was quantified in >50 randomly chosen cells in the whole cell (left panel) and in the nucleus (right panel) under baseline conditions (unstimulated) and after stimulation with IL-6 for 30 minutes (min). Fluorescence intensity was quantified on a scale from zero (i.e. no intensity) to 255 (i.e. maximum intensity). (FIG. 11E) The mean fluorescence of pSTAT3 (Y705) staining as shown in C was quantified in >50 randomly chosen cells in the whole cell (left panel) and in the nucleus (right panel) under baseline conditions (unstimulated) and after stimulation with IL-6 for 30 minutes (min) as in D. (FIG. 11F) EL4 cells were co-transfected with pGL3-Il17a and Renilla luciferase reporter constructs and different combinations of pCMV-Stat3, pCMV-Stat1, pCMV-Fas. Luciferase activity was read after 48 hours of culture and normalized to Renilla activity. Data were normalized between experiments as fold-change versus empty vector control and are merged from seven independent experiments. (FIG. 11G) Sorted naïve CD4⁺ T cells were activated with plate-bound anti-CD3 and anti-CD28 antibodies (each at 2 μg/ml) in the presence of multimerized recombinant Fas ligand (100 ng/ml) or vehicle. The expression of Stat1 was quantified by qPCR. One of three independent experiments with quadruplicate wells is shown. NS not significant, *p<0.05, **p<0.01, ***p<0.005.

FIGS. 12A-12F—Deficiency or inhibition of Fas ligand partially replicates the phenotype induced by Fas deficiency. (FIG. 12A) Sorted naïve CD4⁺CD62L^{high}CD44^{low}CD25⁻ T cells from wildtype (WT) mice were stimulated with the indicated cytokines for various durations and the expression of the Fasl gene encoding Fas ligand was measured by quantitative PCR (qPCR). (B, C) Sorted naïve WT CD4⁺ T cells were cultured in the presence of the indicated cytokines for 96 hours and the expression of Fasl was quantified by qPCR (FIG. 12B) and the proportion of Fas⁺CD4⁺ cells was quantified by flow cytometry (FIG. 12C). (FIG. 12D) Sorted naïve CD4⁺ T cells from WT and Fast mice were cultured in the presence of TGF-β1, IL-6 or IL-1β, IL-6, IL-23 or IL-12 for 4 days and analyzed by intracellular cytokine staining. (FIG. 12E) Sorted naïve CD4⁺ T cells from WT mice were differentiated as in D while adding either H₂O or Kp7-6 (1 mM) dissolved in H2O. Cells were analyzed by intracellular cytokine staining. Data in D-E are representative of 4 independent experiments. (FIG. 12F) Scheme illustrating the proposed mechanistic link between Fas and STAT signalling.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to Meyer Zu Horste, et al., Fas Promotes T Helper 17 Cell Differentiation and Inhibits T Helper 1 Cell Development by Binding and Sequestering Transcription Factor STAT1, Immunity. 2018 Mar. 20; 48(3):556-569.e7. doi: 10.1016/j.immuni.2018.03.008. All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compositions and methods related to FAS-STAT1 interactions. Specifically, provided are cells modulated to have altered FAS-STAT1 and methods of treatment that take advantage of the discovery that FAS-STAT1 interactions can regulate T cell balance. Here, Applicants have demonstrated that Fas promoted the differentiation of Th17 cells and their ability to induce autoimmunity not by affecting apoptosis, but by promoting their stability and preventing their differentiation into Th1-like cells. Applicants used a computational network analysis on expression profiles from wildtype and Fas-deficient cells to nominate STAT1 as the downstream transcription factor mediating effects of Fas in Th17 cells. Applicants experimentally validated that Fas indeed directly bound to STAT1 and thus prevented its excessive activation in response to the Th17 cell-differentiating cytokine IL-6 and enhanced Th17 cell stability. Fas thus reciprocally regulates the balance between competing differentiation programs of T helper cells by regulating the availability of the opposing STAT1 vs. STAT3 proteins. This elucidates an apoptosis-independent function by which Fas controls antagonistic T cell differentiation programs and autoimmunity.

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

Depending on the cytokines used for differentiation, in vitro polarized Th17 cells can either cause severe autoimmune responses upon adoptive transfer (‘pathogenic Th17 cells’) or have little or no effect in inducing autoimmune disease (‘non-pathogenic cells’) (Ghoreschi et al., 2010; and Lee et al., 2012 “Induction and molecular signature of pathogenic Th17 cells,” Nature Immunology, vol. 13(10): 991-999). In vitro differentiation of naïve CD4 T cells in the presence of TGF-β1+IL-6 induces an IL-17A and IL-10 producing population of Th17 cells, that are generally nonpathogenic, whereas activation of naïve T cells in the presence of IL-1β+IL-6+IL-23 or TGF-β3+IL-6 induces a T cell population that produces IL-17A and IFN-γ, and are potent inducers of autoimmune disease induction (Ghoreschi et al., 2010, Lee et al., 2012).

As used herein, terms such as “pathogenic Th17 cell” and/or “pathogenic Th17 phenotype” and all grammatical variations thereof refer to Th17 cells that, exhibit a distinct pathogenic signature where one or more of the following genes or products of these genes is upregulated in Th17 cells: Cxc13, Il22, Il3, Cc14, Gzmb, Lrmp, Cc15, Casp1, Csf2, Cc13, Tbx21, Icos, I17r, Stat4, Lgals3 or Lag3. In certain embodiments, the pathogenic signature is elevated in TGF-β3-induced Th17 cells as compared to TGF-β1-induced Th17 cells. As used herein, terms such as “non-pathogenic Th17 cell” and/or “non-pathogenic Th17 phenotype” and all grammatical variations thereof refer to Th17 cells that exhibit a distinct non-pathogenic signature where one or more of the following genes or products of these genes is up-regulated in Th17 cells: Il6st, Il1rn, lkzf3, Maf, Ahr, 119 or 1110. In certain embodiments, the non-pathogenic signature is elevated in TGF-β1-induced Th17 cells as compared to TGF-β3-induced Th17 cells. In certain embodiments, when induced in the presence of TGF-β3, Th17 cells express a decreased level of one or more genes selected from IL6st, ILlrn, Ikzf3, Maf, Ahr, IL9 and IL10, as compared to the level of expression in TGF-β1-induced Th17 cells.

A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p 1413-1427, 3 Dec. 2015; Gaublomme et al., Single-Cell Genomics Unveils Critical Regulators of Th17 Cell Pathogenicity, Cell Volume 163, Issue 6, p 1400-1412, 3 Dec. 2015; and Internationational publication numbers WO2016138488A2, WO2015130968, WO/2012/048265, WO/2014/145631 and WO/2014/134351, the contents of which are hereby incorporated by reference in their entirety).

As used herein, Fas cell surface death receptor (FAS) may refer to the gene or polypeptide according to the Refseq identifiers NM_152871.3, NM_000043.5, NM_001320619.1 and NM_152872.3.

As used herein, signal transducer and activator of transcription 1 (STAT1) may refer to the gene or polypeptide according to the Refseq identifiers NM_007315.3 and NM_139266.2.

Although the exemplary gene sequences set forth above are for the human genes, and thus are best suited for use in human cells, one of skill in the art could readily identify mammalian homologs using database searches (for known sequences) or routine molecular biological techniques (to identify additional sequences). In general, genes are considered homologs if they show at least 80%, e.g., 90%, 95%, or more, identity in conserved regions (e.g., biologically important regions).

As used herein, “modulating” or “to modulate” generally means either reducing or inhibiting the expression or activity of, or alternatively increasing the expression or activity of a target or antigen (e.g., FAS, STAT1). In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen, such as FAS and/or STAT1 binding. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that targets expression of a gene, such as but not limited to a DNA targeting agent (e.g., CRISPR system, TALE, Zinc finger protein) or RNA targeting agent (e.g., CRISPR system, inhibitory nucleic acid molecules such as RNAi, miRNA, ribozyme).

### Adoptive Cell Transfer

In certain embodiments, T cells used for adoptive cell therapy are modified to comprise altered FAS-STAT1 binding. In certain embodiments, altering FAS-STAT1 binding can provide for less pathogenic T cells, more pathogenic T cells, or suppressive T cells. Not being bound by a theory, non-pathogenic or suppressive T cells may dampen an immune response when transferred to a subject in need thereof (e.g., to treat an autoimmune or inflammatory disease). Not being bound by a theory, pathogenic T cells may target and eliminate tumor cells when transferred to a subject in need thereof. In certain embodiments, T cells used for adoptive cell therapy are modified to comprise altered FAS-STAT1 binding in addition to any modifications described herein. In certain embodiments, the T cells are specific for an antigen as described further herein. In certain embodiments, T cells are modified to express an endogenous TCR or CAR specific for a tumor antigen as described herein.

As used herein, “ACT”, “adoptive cell therapy” and “adoptive cell transfer” may be used interchangeably. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

Aspects of the invention involve the adoptive transfer of immune system cells, such as T cells, specific for selected antigens, such as tumor associated antigens or tumor specific neoantigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDG1cp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p 190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WTI), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia. For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer.

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3):

(SEQ ID NO: 1))
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS.

Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO:2) and continuing all the way to the carboxy-terminus of the protein. The sequence is reproduced herein:

(SEQ ID NO: 3)
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex isdestabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3ζ chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CART cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2016, Multiplex genome editing to generate universal CAR T cells resistant to PD1 inhibition, Clin Cancer Res. 2016 Nov. 4; and Qasim et al., 2017, Molecular remission of infant B-ALL after infusion of universal TALEN gene-edited CAR T cells, Sci Transl Med. 2017 Jan. 25; 9(374)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CART cells or any cells used for adoptive cell transfer may be edited. Editing may be performed for example to insert or knock-in an exogenous gene, such as an exogenous gene encoding a CAR or a TCR, at a preselected locus in a cell; to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

By inactivating a gene it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci.

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment T cells that have infiltrated a tumor are isolated. T cells may be removed during surgery. T cells may be isolated after removal of tumor tissue by biopsy. T cells may be isolated by any means known in the art. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MEW tetramers (Altman, et al., Science. 1996 Oct. 4; 274(5284):94-6). In another embodiment the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-WIC tetramers can be generated using techniques known in the art and can be made with any WIC molecule of interest and any antigen of interest as described herein. Specific epitopes to be used in this context can be identified using numerous assays known in the art. For example, the ability of a polypeptide to bind to WIC class I may be evaluated indirectly by monitoring the ability to promote incorporation of 125I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one T cells are isolated by contacting the T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Genetic Modifying Agents

In certain embodiments, the one or more modulating agents may be a genetic modifying agent. The genetic modifying agent may comprise a CRISPR system, a zinc finger nuclease system, a TALEN, or a meganuclease.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters—especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemicially modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemicially modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the compelementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programming of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, 02 concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/r52), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extented length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g., Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient. In one embodiment, the modified cell for cell therapy is a CAR-T cell capable of recognizing and/or attacking a tumor cell.

### Cytosine Deaminase

Programmable deamination of cytosine has been reported and may be used for correction of A→G and T→C point mutations. For example, Komor et al., Nature (2016) 533:420-424 reports targeted deamination of cytosine by APOBEC1 cytidine deaminase in a non-targeted DNA stranded displaced by the binding of a Cas9-guide RNA complex to a targeted DNA strand, which results in conversion of cytosine to uracil. See also Kim et al., Nature Biotechnology (2017) 35:371-376; Shimatani et al., Nature Biotechnology (2017) doi:10.1038/nbt.3833; Zong et al., Nature Biotechnology (2017) doi:10.1038/nbt.3811; Yang Nature Communication (2016) doi:10.1038/ncomms13330.

### Adenosine Deaminase

The term “adenosine deaminase” or “adenosine deaminase protein” as used herein refers to a protein, a polypeptide, or one or more functional domain(s) of a protein or a polypeptide that is capable of catalyzing a hydrolytic deamination reaction that converts an adenine (or an adenine moiety of a molecule) to a hypoxanthine (or a hypoxanthine moiety of a molecule), as shown below. In some embodiments, the adenine-containing molecule is an adenosine (A), and the hypoxanthine-containing molecule is an inosine (I). The adenine-containing molecule can be deoxyribonucleic acid (DNA) or ribonucleic acid (RNA).

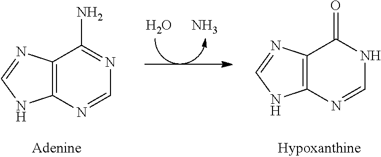

According to the present disclosure, adenosine deaminases that can be used in connection with the present disclosure include, but are not limited to, members of the enzyme family known as adenosine deaminases that act on RNA (ADARs), members of the enzyme family known as adenosine deaminases that act on tRNA (ADATs), and other adenosine deaminase domain-containing (ADAD) family members. According to the present disclosure, the adenosine deaminase is capable of targeting adenine in a RNA/DNA heteroduplex. Indeed, Zheng et al. (Nucleic Acids Res. 2017, 45(6): 3369-3377) has demonstrated that ADARs can carry out adenosine to inosine editing reactions on RNA/DNA heteroduplexes. In particular embodiments, the adenosine deaminase has been modified to increase its ability to edit DNA in a RNA/DNA heteroduplex as detailed herein.

In some embodiments, the adenosine deaminase is derived from one or more metazoa species, including but not limited to, mammals, birds, frogs, squids, fish, flies and worms. In some embodiments, the adenosine deaminase is a human, squid or Drosophila adenosine deaminase.

In some embodiments, the adenosine deaminase is a human ADAR, including hADAR1, hADAR2, hADAR3. In some embodiments, the adenosine deaminase is a Caenorhabditis elegans ADAR protein, including ADR-1 and ADR-2. In some embodiments, the adenosine deaminase is a Drosophila ADAR protein, including dAdar. In some embodiments, the adenosine deaminase is a squid Loligo pealeii ADAR protein, including sqADAR2a and sqADAR2b. In some embodiments, the adenosine deaminase is a human ADAT protein. In some embodiments, the adenosine deaminase is a Drosophila ADAT protein. In some embodiments, the adenosine deaminase is a human ADAD protein, including TENR (hADAD1) and TENRL (hADAD2).

In some embodiments, the adenosine deaminase protein recognizes and converts one or more target adenosine residue(s) in a double-stranded nucleic acid substrate into inosine residues (s). In some embodiments, the double-stranded nucleic acid substrate is a RNA-DNA hybrid duplex. In some embodiments, the adenosine deaminase protein recognizes a binding window on the double-stranded substrate. In some embodiments, the binding window contains at least one target adenosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the adenosine deaminase protein comprises one or more deaminase domains. Not intended to be bound by theory, it is contemplated that the deaminase domain functions to recognize and convert one or more target adenosine (A) residue(s) contained in a double-stranded nucleic acid substrate into inosine (I) residues (s). In some embodiments, the deaminase domain comprises an active center. In some embodiments, the active center comprises a zinc ion. In some embodiments, during the A-to-I editing process, base pairing at the target adenosine residue is disrupted, and the target adenosine residue is “flipped” out of the double helix to become accessible by the adenosine deaminase. In some embodiments, amino acid residues in or near the active center interact with one or more nucleotide(s) 5′ to a target adenosine residue. In some embodiments, amino acid residues in or near the active center interact with one or more nucleotide(s) 3′ to a target adenosine residue. In some embodiments, amino acid residues in or near the active center further interact with the nucleotide complementary to the target adenosine residue on the opposite strand. In some embodiments, the amino acid residues form hydrogen bonds with the 2′ hydroxyl group of the nucleotides.

In some embodiments, the adenosine deaminase comprises human ADAR2 full protein (hADAR2) or the deaminase domain thereof (hADAR2-D). In some embodiments, the adenosine deaminase is an ADAR family member that is homologous to hADAR2 or hADAR2-D.

Particularly, in some embodiments, the homologous ADAR protein is human ADAR1 (hADAR1) or the deaminase domain thereof (hADAR1-D). In some embodiments, glycine 1007 of hADAR1-D corresponds to glycine⁴⁸⁷ hADAR2-D, and glutamic Acid¹⁰⁰⁸ of hADAR1-D corresponds to glutamic acid⁴⁸⁸ of hADAR2-D.

In some embodiments, the adenosine deaminase comprises the wild-type amino acid sequence of hADAR2-D. In some embodiments, the adenosine deaminase comprises one or more mutations in the hADAR2-D sequence, such that the editing efficiency, and/or substrate editing preference of hADAR2-D is changed according to specific needs.

Certain mutations of hADAR1 and hADAR2 proteins have been described in Kuttan et al., Proc Natl Acad Sci USA. (2012) 109(48):E3295-304; Want et al. ACS Chem Biol. (2015) 10(11):2512-9; and Zheng et al. Nucleic Acids Res. (2017) 45(6):3369-337, each of which is incorporated herein by reference in its entirety.

In some embodiments, the adenosine deaminase comprises a mutation at glycine³³⁶ of the hADAR2-D amino acid sequence, or a corresponding position in a homologous ADAR protein. In some embodiments, the glycine residue at position 336 is replaced by an aspartic acid residue (G336D).

In some embodiments, the adenosine deaminase comprises a mutation at Glycine⁴⁸⁷ of the hADAR2-D amino acid sequence, or a corresponding position in a homologous ADAR protein. In some embodiments, the glycine residue at position 487 is replaced by a non-polar amino acid residue with relatively small side chains. For example, in some embodiments, the glycine residue at position 487 is replaced by an alanine residue (G487A). In some embodiments, the glycine residue at position 487 is replaced by a valine residue (G487V). In some embodiments, the glycine residue at position 487 is replaced by an amino acid residue with relatively large side chains. In some embodiments, the glycine residue at position 487 is replaced by a arginine residue (G487R). In some embodiments, the glycine residue at position 487 is replaced by a lysine residue (G487K). In some embodiments, the glycine residue at position 487 is replaced by a tryptophan residue (G487W). In some embodiments, the glycine residue at position 487 is replaced by a tyrosine residue (G487Y).

In some embodiments, the adenosine deaminase comprises a mutation at glutamic acid⁴⁸⁸ of the hADAR2-D amino acid sequence, or a corresponding position in a homologous ADAR protein. In some embodiments, the glutamic acid residue at position 488 is replaced by a glutamine residue (E488Q). In some embodiments, the glutamic acid residue at position 488 is replaced by a histidine residue (E488H). In some embodiments, the glutamic acid residue at position 488 is replace by an arginine residue (E488R). In some embodiments, the glutamic acid residue at position 488 is replace by a lysine residue (E488K). In some embodiments, the glutamic acid residue at position 488 is replace by an asparagine residue (E488N). In some embodiments, the glutamic acid residue at position 488 is replace by an alanine residue (E488A). In some embodiments, the glutamic acid residue at position 488 is replace by a Methionine residue (E488M). In some embodiments, the glutamic acid residue at position 488 is replace by a serine residue (E488S). In some embodiments, the glutamic acid residue at position 488 is replace by a phenylalanine residue (E488F). In some embodiments, the glutamic acid residue at position 488 is replace by a lysine residue (E488L). In some embodiments, the glutamic acid residue at position 488 is replace by a tryptophan residue (E488W).

In some embodiments, the adenosine deaminase comprises a mutation at threonine⁴⁹⁰ of the hADAR2-D amino acid sequence, or a corresponding position in a homologous ADAR protein. In some embodiments, the threonine residue at position 490 is replaced by a cysteine residue (T490C). In some embodiments, the threonine residue at position 490 is replaced by a serine residue (T490S). In some embodiments, the threonine residue at position 490 is replaced by an alanine residue (T490A). In some embodiments, the threonine residue at position 490 is replaced by a phenylalanine residue (T490F). In some embodiments, the threonine residue at position 490 is replaced by a tyrosine residue (T490Y). In some embodiments, the threonine residue at position 490 is replaced by a serine residue (T490R). In some embodiments, the threonine residue at position 490 is replaced by an alanine residue (T490K). In some embodiments, the threonine residue at position 490 is replaced by a phenylalanine residue (T490P). In some embodiments, the threonine residue at position 490 is replaced by a tyrosine residue (T490E).

In some embodiments, the adenosine deaminase comprises a mutation at valine⁴⁹³ of the hADAR2-D amino acid sequence, or a corresponding position in a homologous ADAR protein. In some embodiments, the valine residue at position 493 is replaced by an alanine residue (V493A). In some embodiments, the valine residue at position 493 is replaced by a serine residue (V493S). In some embodiments, the valine residue at position 493 is replaced by a threonine residue (V493T). In some embodiments, the valine residue at position 493 is replaced by an arginine residue (V493R). In some embodiments, the valine residue at position 493 is replaced by an aspartic acid residue (V493D). In some embodiments, the valine residue at position 493 is replaced by a proline residue (V493P). In some embodiments, the valine residue at position 493 is replaced by a glycine residue (V493G).

In some embodiments, the adenosine deaminase comprises a mutation at alanine⁵⁸⁹ of the hADAR2-D amino acid sequence, or a corresponding position in a homologous ADAR protein. In some embodiments, the alanine residue at position 589 is replaced by a valine residue (A589V).

In some embodiments, the adenosine deaminase comprises a mutation at asparagine⁵⁹⁷ of the hADAR2-D amino acid sequence, or a corresponding position in a homologous ADAR protein. In some embodiments, the asparagine residue at position 597 is replaced by a lysine residue (N597K). In some embodiments, the adenosine deaminase comprises a mutation at position 597 of the amino acid sequence, which has an asparagine residue in the wild type sequence. In some embodiments, the asparagine residue at position 597 is replaced by an arginine residue (N597R). In some embodiments, the adenosine deaminase comprises a mutation at position 597 of the amino acid sequence, which has an asparagine residue in the wild type sequence. In some embodiments, the asparagine residue at position 597 is replaced by an alanine residue (N597A). In some embodiments, the adenosine deaminase comprises a mutation at position 597 of the amino acid sequence, which has an asparagine residue in the wild type sequence. In some embodiments, the asparagine residue at position 597 is replaced by a glutamic acid residue (N597E). In some embodiments, the adenosine deaminase comprises a mutation at position 597 of the amino acid sequence, which has an asparagine residue in the wild type sequence. In some embodiments, the asparagine residue at position 597 is replaced by a histidine residue (N597H). In some embodiments, the adenosine deaminase comprises a mutation at position 597 of the amino acid sequence, which has an asparagine residue in the wild type sequence. In some embodiments, the asparagine residue at position 597 is replaced by a glycine residue (N597G). In some embodiments, the adenosine deaminase comprises a mutation at position 597 of the amino acid sequence, which has an asparagine residue in the wild type sequence. In some embodiments, the asparagine residue at position 597 is replaced by a tyrosine residue (N597Y). In some embodiments, the asparagine residue at position 597 is replaced by a phenylalanine residue (N597F).

In some embodiments, the adenosine deaminase comprises a mutation at serine⁵⁹⁹ of the hADAR2-D amino acid sequence, or a corresponding position in a homologous ADAR protein. In some embodiments, the serine residue at position 599 is replaced by a threonine residue (S599T).

In some embodiments, the adenosine deaminase comprises a mutation at asparagine⁶¹³ of the hADAR2-D amino acid sequence, or a corresponding position in a homologous ADAR protein. In some embodiments, the asparagine residue at position 613 is replaced by a lysine residue (N613K). In some embodiments, the adenosine deaminase comprises a mutation at position 613 of the amino acid sequence, which has an asparagine residue in the wild type sequence. In some embodiments, the asparagine residue at position 613 is replaced by an arginine residue (N613R). In some embodiments, the adenosine deaminase comprises a mutation at position 613 of the amino acid sequence, which has an asparagine residue in the wild type sequence. In some embodiments, the asparagine residue at position 613 is replaced by an alanine residue (N613A) In some embodiments, the adenosine deaminase comprises a mutation at position 613 of the amino acid sequence, which has an asparagine residue in the wild type sequence. In some embodiments, the asparagine residue at position 613 is replaced by a glutamic acid residue (N613E).

In some embodiments, to improve editing efficiency, the adenosine deaminase may comprise one or more of the mutations: G336D, G487A, G487V, E488Q, E488H, E488R, E488N, E488A, E488S, E488M, T490C, T490S, V493T, V493S, V493A, V493R, V493D, V493P, V493G, N597K, N597R, N597A, N597E, N597H, N597G, N597Y, A589V, S599T, N613K, N613R, N613A, N613E, based on amino acid sequence positions of hADAR2-D, and mutations in a homologous ADAR protein corresponding to the above.

In some embodiments, to reduce editing efficiency, the adenosine deaminase may comprise one or more of the mutations: E488F, E488L, E488W, T490A, T490F, T490Y, T490R, T490K, T490P, T490E, N597F, based on amino acid sequence positions of hADAR2-D, and mutations in a homologous ADAR protein corresponding to the above. In particular embodiments, it can be of interest to use an adenosine deaminase enzyme with reduced efficacy to reduce off-target effects.

The terms “editing specificity” and “editing preference” are used interchangeably herein to refer to the extent of A-to-I editing at a particular adenosine site in a double-stranded substrate. In some embodiment, the substrate editing preference is determined by the 5′ nearest neighbor and/or the 3′ nearest neighbor of the target adenosine residue. In some embodiments, the adenosine deaminase has preference for the 5′ nearest neighbor of the substrate ranked as U>A>C>G (“>” indicates greater preference). In some embodiments, the adenosine deaminase has preference for the 3′ nearest neighbor of the substrate ranked as G>C˜A>U (“>” indicates greater preference; “˜” indicates similar preference). In some embodiments, the adenosine deaminase has preference for the 3′ nearest neighbor of the substrate ranked as G>C>U˜A (“>” indicates greater preference; “˜” indicates similar preference). In some embodiments, the adenosine deaminase has preference for the 3′ nearest neighbor of the substrate ranked as G>C>A>U (“>” indicates greater preference). In some embodiments, the adenosine deaminase has preference for the 3′ nearest neighbor of the substrate ranked as C˜G˜A>U (“>” indicates greater preference; “˜” indicates similar preference). In some embodiments, the adenosine deaminase has preference for a triplet sequence containing the target adenosine residue ranked as TAG>AAG>CAC>AAT>GAA>GAC (“>” indicates greater preference), the center A being the target adenosine residue.

In some embodiments, the substrate editing preference of an adenosine deaminase is affected by the presence or absence of a nucleic acid binding domain in the adenosine deaminase protein. In some embodiments, to modify substrate editing preference, the deaminase domain is connected with a double-strand RNA binding domain (dsRBD) or a double-strand RNA binding motif (dsRBM). In some embodiments, the dsRBD or dsRBM may be derived from an ADAR protein, such as hADAR1 or hADAR2. In some embodiments, a full length ADAR protein that comprises at least one dsRBD and a deaminase domain is used. In some embodiments, the one or more dsRBM or dsRBD is at the N-terminus of the deaminase domain. In other embodiments, the one or more dsRBM or dsRBD is at the C-terminus of the deaminase domain.

In some embodiments, the substrate editing preference of an adenosine deaminase is affected by amino acid residues near or in the active center of the enzyme. In some embodiments, to modify substrate editing preference, the adenosine deaminase may comprise one or more of the mutations: G336D, G487R, G487K, G487W, G487Y, E488Q, E488N, T490A, V493A, V493T, V493S, N597K, N597R, A589V, S599T, N613K, N613R, based on amino acid sequence positions of hADAR2-D, and mutations in a homologous ADAR protein corresponding to the above.

Particularly, in some embodiments, to reduce editing specificity, the adenosine deaminase can comprise one or more of mutations E488Q, V493A, N597K, N613K, based on amino acid sequence positions of hADAR2-D, and mutations in a homologous ADAR protein corresponding to the above. In some embodiments, to increase editing specificity, the adenosine deaminase can comprise mutation T490A.

In some embodiments, to increase editing preference for target adenosine (A) with an immediate 5′ G, such as substrates comprising the triplet sequence GAC, the center A being the target adenosine residue, the adenosine deaminase can comprise one or more of mutations G336D, E488Q, E488N, V493T, V493S, V493A, A589V, N597K, N597R, S599T, N613K, N613R, based on amino acid sequence positions of hADAR2-D, and mutations in a homologous ADAR protein corresponding to the above.

Particularly, in some embodiments, the adenosine deaminase comprises mutation E488Q or a corresponding mutation in a homologous ADAR protein for editing substrates comprising the following triplet sequences: GAC, GAA, GAU, GAG, CAU, AAU, UAC, the center A being the target adenosine residue.

In some embodiments, to reduce off-target effects, the adenosine deaminase comprises one or more of mutations at R348, V351, T375, K376, E396, C451, R455, N473, R474, K475, R477, R481, S486, E488, T490, S495, R510, based on amino acid sequence positions of hADAR2-D, and mutations in a homologous ADAR protein corresponding to the above. In some embodiments, the adenosine deaminase comprises mutation at E488 and one or more additional positions selected from R348, V351, T375, K376, E396, C451, R455, N473, R474, K475, R477, R481, S486, T490, S495, R510. In some embodiments, the adenosine deaminase comprises mutation at T375, and optionally at one or more additional positions. In some embodiments, the adenosine deaminase comprises mutation at N473, and optionally at one or more additional positions. In some embodiments, the adenosine deaminase comprises mutation at V351, and optionally at one or more additional positions. In some embodiments, the adenosine deaminase comprises mutation at E488 and T375, and optionally at one or more additional positions. In some embodiments, the adenosine deaminase comprises mutation at E488 and N473, and optionally at one or more additional positions. In some embodiments, the adenosine deaminase comprises mutation E488 and V351, and optionally at one or more additional positions. In some embodiments, the adenosine deaminase comprises mutation at E488 and one or more of T375, N473, and V351.

In some embodiments, to reduce off-target effects, the adenosine deaminase comprises one or more of mutations selected from R348E, V351L, T375G, T375S, R455G, R455S, R455E, N473D, R474E, K475Q, R477E, R481E, S486T, E488Q, T490A, T490S, S495T, and R510E, based on amino acid sequence positions of hADAR2-D, and mutations in a homologous ADAR protein corresponding to the above. In some embodiments, the adenosine deaminase comprises mutation E488Q and one or more additional mutations selected from R348E, V351L, T375G, T375S, R455G, R455S, R455E, N473D, R474E, K475Q, R477E, R481E, S486T, T490A, T490S, S495T, and R510E. In some embodiments, the adenosine deaminase comprises mutation T375G or T375S, and optionally one or more additional mutations. In some embodiments, the adenosine deaminase comprises mutation N473D, and optionally one or more additional mutations. In some embodiments, the adenosine deaminase comprises mutation V351L, and optionally one or more additional mutations. In some embodiments, the adenosine deaminase comprises mutation E488Q, and T375G or T375G, and optionally one or more additional mutations. In some embodiments, the adenosine deaminase comprises mutation E488Q and N473D, and optionally one or more additional mutations. In some embodiments, the adenosine deaminase comprises mutation E488Q and V351L, and optionally one or more additional mutations. In some embodiments, the adenosine deaminase comprises mutation E488Q and one or more of T375G/S, N473D and V351L.

Crystal structures of the human ADAR2 deaminase domain bound to duplex RNA reveal a protein loop that binds the RNA on the 5′ side of the modification site. This 5′ binding loop is one contributor to substrate specificity differences between ADAR family members. See Wang et al., Nucleic Acids Res., 44(20):9872-9880 (2016), the content of which is incorporated herein by reference in its entirety. In addition, an ADAR2-specific RNA-binding loop was identified near the enzyme active site. See Mathews et al., Nat. Struct. Mol. Biol., 23(5):426-33 (2016), the content of which is incorporated herein by reference in its entirety. In some embodiments, the adenosine deaminase comprises one or more mutations in the RNA binding loop to improve editing specificity and/or efficiency.

In some embodiments, the adenosine deaminase comprises a mutation at alanine⁴⁵⁴ of the hADAR2-D amino acid sequence, or a corresponding position in a homologous ADAR protein. In some embodiments, the alanine residue at position 454 is replaced by a serine residue (A4545). In some embodiments, the alanine residue at position 454 is replaced by a cysteine residue (A454C). In some embodiments, the alanine residue at position 454 is replaced by an aspartic acid residue (A454D).

In some embodiments, the adenosine deaminase comprises a mutation at arginine⁴⁵⁵ of the hADAR2-D amino acid sequence, or a corresponding position in a homologous ADAR protein. In some embodiments, the arginine residue at position 455 is replaced by an alanine residue (R455A). In some embodiments, the arginine residue at position 455 is replaced by a valine residue (R455V). In some embodiments, the arginine residue at position 455 is replaced by a histidine residue (R455H). In some embodiments, the arginine residue at position 455 is replaced by a glycine residue (R455G). In some embodiments, the arginine residue at position 455 is replaced by a serine residue (R455S). In some embodiments, the arginine residue at position 455 is replaced by a glutamic acid residue (R455E).

In some embodiments, the adenosine deaminase comprises a mutation at isoleucine⁴⁵⁶ of the hADAR2-D amino acid sequence, or a corresponding position in a homologous ADAR protein. In some embodiments, the isoleucine residue at position 456 is replaced by a valine residue (I456V). In some embodiments, the isoleucine residue at position 456 is replaced by a leucine residue (I456L). In some embodiments, the isoleucine residue at position 456 is replaced by an aspartic acid residue (I456D).

In some embodiments, the adenosine deaminase comprises a mutation at phenylalanine⁴⁵⁷ of the hADAR2-D amino acid sequence, or a corresponding position in a homologous ADAR protein. In some embodiments, the phenylalanine residue at position 457 is replaced by a tyrosine residue (F457Y). In some embodiments, the phenylalanine residue at position 457 is replaced by an arginine residue (F457R). In some embodiments, the phenylalanine residue at position 457 is replaced by a glutamic acid residue (F457E).

In some embodiments, the adenosine deaminase comprises a mutation at serine⁴⁵⁸ of the hADAR2-D amino acid sequence, or a corresponding position in a homologous ADAR protein. In some embodiments, the serine residue at position 458 is replaced by a valine residue (S458V). In some embodiments, the serine residue at position 458 is replaced by a phenylalanine residue (S458F). In some embodiments, the serine residue at position 458 is replaced by a proline residue (S458P).

In some embodiments, the adenosine deaminase comprises a mutation at proline⁴⁵⁹ of the hADAR2-D amino acid sequence, or a corresponding position in a homologous ADAR protein. In some embodiments, the proline residue at position 459 is replaced by a cysteine residue (P459C). In some embodiments, the proline residue at position 459 is replaced by a histidine residue (P459H). In some embodiments, the proline residue at position 459 is replaced by a tryptophan residue (P459W).

In some embodiments, the adenosine deaminase comprises a mutation at histidine⁴⁶⁰ of the hADAR2-D amino acid sequence, or a corresponding position in a homologous ADAR protein. In some embodiments, the histidine residue at position 460 is replaced by an arginine residue (H460R). In some embodiments, the histidine residue at position 460 is replaced by an isoleucine residue (H460I). In some embodiments, the histidine residue at position 460 is replaced by a proline residue (H460P).

In some embodiments, the adenosine deaminase comprises a mutation at proline⁴⁶² of the hADAR2-D amino acid sequence, or a corresponding position in a homologous ADAR protein. In some embodiments, the proline residue at position 462 is replaced by a serine residue (P462S). In some embodiments, the proline residue at position 462 is replaced by a tryptophan residue (P462W). In some embodiments, the proline residue at position 462 is replaced by a glutamic acid residue (P462E).

In some embodiments, the adenosine deaminase comprises a mutation at aspartic acid⁴⁶⁹ of the hADAR2-D amino acid sequence, or a corresponding position in a homologous ADAR protein. In some embodiments, the aspartic acid residue at position 469 is replaced by a glutamine residue (D469Q). In some embodiments, the aspartic acid residue at position 469 is replaced by a serine residue (D469S). In some embodiments, the aspartic acid residue at position 469 is replaced by a tyrosine residue (D469Y).

In some embodiments, the adenosine deaminase comprises a mutation at arginine⁴⁷⁰ of the hADAR2-D amino acid sequence, or a corresponding position in a homologous ADAR protein. In some embodiments, the arginine residue at position 470 is replaced by an alanine residue (R470A). In some embodiments, the arginine residue at position 470 is replaced by an isoleucine residue (R470I). In some embodiments, the arginine residue at position 470 is replaced by an aspartic acid residue (R470D).

In some embodiments, the adenosine deaminase comprises a mutation at histidine⁴⁷¹ of the hADAR2-D amino acid sequence, or a corresponding position in a homologous ADAR protein. In some embodiments, the histidine residue at position 471 is replaced by a lysine residue (H471K). In some embodiments, the histidine residue at position 471 is replaced by a threonine residue (H471T). In some embodiments, the histidine residue at position 471 is replaced by a valine residue (H471V).

In some embodiments, the adenosine deaminase comprises a mutation at proline⁴⁷² of the hADAR2-D amino acid sequence, or a corresponding position in a homologous ADAR protein. In some embodiments, the proline residue at position 472 is replaced by a lysine residue (P472K). In some embodiments, the proline residue at position 472 is replaced by a threonine residue (P472T). In some embodiments, the proline residue at position 472 is replaced by an aspartic acid residue (P472D).

In some embodiments, the adenosine deaminase comprises a mutation at asparagine⁴⁷³ of the hADAR2-D amino acid sequence, or a corresponding position in a homologous ADAR protein. In some embodiments, the asparagine residue at position 473 is replaced by an arginine residue (N473R). In some embodiments, the asparagine residue at position 473 is replaced by a tryptophan residue (N473W). In some embodiments, the asparagine residue at position 473 is replaced by a proline residue (N473P). In some embodiments, the asparagine residue at position 473 is replaced by an aspartic acid residue (N473D).

In some embodiments, the adenosine deaminase comprises a mutation at arginine⁴⁷⁴ of the hADAR2-D amino acid sequence, or a corresponding position in a homologous ADAR protein. In some embodiments, the arginine residue at position 474 is replaced by a lysine residue (R474K). In some embodiments, the arginine residue at position 474 is replaced by a glycine residue (R474G). In some embodiments, the arginine residue at position 474 is replaced by an aspartic acid residue (R474D). In some embodiments, the arginine residue at position 474 is replaced by a glutamic acid residue (R474E).

In some embodiments, the adenosine deaminase comprises a mutation at lysine⁴⁷⁵ of the hADAR2-D amino acid sequence, or a corresponding position in a homologous ADAR protein. In some embodiments, the lysine residue at position 475 is replaced by a glutamine residue (K475Q). In some embodiments, the lysine residue at position 475 is replaced by an asparagine residue (K475N). In some embodiments, the lysine residue at position 475 is replaced by an aspartic acid residue (K475D).

In some embodiments, the adenosine deaminase comprises a mutation at alanine⁴⁷⁶ of the hADAR2-D amino acid sequence, or a corresponding position in a homologous ADAR protein. In some embodiments, the alanine residue at position 476 is replaced by a serine residue (A476S). In some embodiments, the alanine residue at position 476 is replaced by an arginine residue (A476R). In some embodiments, the alanine residue at position 476 is replaced by a glutamic acid residue (A476E).

In some embodiments, the adenosine deaminase comprises a mutation at arginine⁴⁷⁷ of the hADAR2-D amino acid sequence, or a corresponding position in a homologous ADAR protein. In some embodiments, the arginine residue at position 477 is replaced by a lysine residue (R477K). In some embodiments, the arginine residue at position 477 is replaced by a threonine residue (R477T). In some embodiments, the arginine residue at position 477 is replaced by a phenylalanine residue (R477F). In some embodiments, the arginine residue at position 474 is replaced by a glutamic acid residue (R477E).

In some embodiments, the adenosine deaminase comprises a mutation at glycine⁴⁷⁸ of the hADAR2-D amino acid sequence, or a corresponding position in a homologous ADAR protein. In some embodiments, the glycine residue at position 478 is replaced by an alanine residue (G478A). In some embodiments, the glycine residue at position 478 is replaced by an arginine residue (G478R). In some embodiments, the glycine residue at position 478 is replaced by a tyrosine residue (G478Y).

In some embodiments, the adenosine deaminase comprises a mutation at glutamine⁴⁷⁹ of the hADAR2-D amino acid sequence, or a corresponding position in a homologous ADAR protein. In some embodiments, the glutamine residue at position 479 is replaced by an asparagine residue (Q479N). In some embodiments, the glutamine residue at position 479 is replaced by a serine residue (Q479S). In some embodiments, the glutamine residue at position 479 is replaced by a proline residue (Q479P).

In some embodiments, the adenosine deaminase comprises a mutation at arginine³⁴⁸ of the hADAR2-D amino acid sequence, or a corresponding position in a homologous ADAR protein. In some embodiments, the arginine residue at position 348 is replaced by an alanine residue (R348A). In some embodiments, the arginine residue at position 348 is replaced by a glutamic acid residue (R348E).

In some embodiments, the adenosine deaminase comprises a mutation at valine³⁵¹ of the hADAR2-D amino acid sequence, or a corresponding position in a homologous ADAR protein. In some embodiments, the valine residue at position 351 is replaced by a leucine residue (V351L).

In some embodiments, the adenosine deaminase comprises a mutation at threonine³⁷⁵ of the hADAR2-D amino acid sequence, or a corresponding position in a homologous ADAR protein. In some embodiments, the threonine residue at position 375 is replaced by a glycine residue (T375G). In some embodiments, the threonine residue at position 375 is replaced by a serine residue (T375S).

In some embodiments, the adenosine deaminase comprises a mutation at arginine⁴⁸¹ of the hADAR2-D amino acid sequence, or a corresponding position in a homologous ADAR protein. In some embodiments, the arginine residue at position 481 is replaced by a glutamic acid residue (R481E).

In some embodiments, the adenosine deaminase comprises a mutation at serine⁴⁸⁶ of the hADAR2-D amino acid sequence, or a corresponding position in a homologous ADAR protein. In some embodiments, the serine residue at position 486 is replaced by a threonine residue (S486T).

In some embodiments, the adenosine deaminase comprises a mutation at threonine⁴⁹⁰ of the hADAR2-D amino acid sequence, or a corresponding position in a homologous ADAR protein. In some embodiments, the threonine residue at position 490 is replaced by an alanine residue (T490A). In some embodiments, the threonine residue at position 490 is replaced by a serine residue (T490S).

In some embodiments, the adenosine deaminase comprises a mutation at serine⁴⁹⁵ of the hADAR2-D amino acid sequence, or a corresponding position in a homologous ADAR protein. In some embodiments, the serine residue at position 495 is replaced by a threonine residue (S495T).

In some embodiments, the adenosine deaminase comprises a mutation at arginine⁵¹⁰ of the hADAR2-D amino acid sequence, or a corresponding position in a homologous ADAR protein. In some embodiments, the arginine residue at position 510 is replaced by a glutamine residue (R510Q). In some embodiments, the arginine residue at position 510 is replaced by an alanine residue (R510A). In some embodiments, the arginine residue at position 510 is replaced by a glutamic acid residue (R510E).

In some embodiments, the adenosine deaminase comprises a mutation at glycine⁵⁹³ of the hADAR2-D amino acid sequence, or a corresponding position in a homologous ADAR protein. In some embodiments, the glycine residue at position 593 is replaced by an alanine residue (G593A). In some embodiments, the glycine residue at position 593 is replaced by a glutamic acid residue (G593E).

In some embodiments, the adenosine deaminase comprises a mutation at lysine⁵⁹⁴ of the hADAR2-D amino acid sequence, or a corresponding position in a homologous ADAR protein. In some embodiments, the lysine residue at position 594 is replaced by an alanine residue (K594A).

In some embodiments, the adenosine deaminase comprises the wild-type amino acid sequence of hADAR1-D (e.g., MGSGGGGSEGAPKKKRKVGSSLGTGNRCVKGDSLSLKGE TVNDCHAEIISRRGFIRFLYSELMKYNSQTAKDSIFEPAKGGEKLQIKKTVSFHLYISTAP CGDGALFDKSCSDRAMESTESRHYPVFENPKQGKLRTKVENGQGTIPVESSDIVPTWDG IRLGERLRTMSCSDKILRWNVLGLQGALLTHFLQPIYLKSVTLGYLFSQGHLTRAICCRV TRDGSAFEDGLRHPFIVNHPKVGRVSIYDSKRQSGKTKETSVNWCLADGYDLEILDGTR GTVDGPRNELSRVSKKNIFLLFKKLCSFRYRRDLLRLSYGEAKKAARDYETAKNYFKKG LKDMGYGNWISKPQEEKNF* (SEQ ID NO:4)). In some embodiments, the adenosine deaminase comprises one or more mutations in the hADAR1-D sequence, such that the editing efficiency, and/or substrate editing preference of hADAR1-D is changed according to specific needs.

In some embodiments, the adenosine deaminase comprises a mutation at Glycine¹⁰⁰⁷ of the hADAR1-D amino acid sequence, or a corresponding position in a homologous ADAR protein. In some embodiments, the glycine residue at position 1007 is replaced by a non-polar amino acid residue with relatively small side chains. For example, in some embodiments, the glycine residue at position 1007 is replaced by an alanine residue (G1007A). In some embodiments, the glycine residue at position 1007 is replaced by a valine residue (G1007V). In some embodiments, the glycine residue at position 1007 is replaced by an amino acid residue with relatively large side chains. In some embodiments, the glycine residue at position 1007 is replaced by an arginine residue (G1007R). In some embodiments, the glycine residue at position 1007 is replaced by a lysine residue (G1007K). In some embodiments, the glycine residue at position 1007 is replaced by a tryptophan residue (G1007W). In some embodiments, the glycine residue at position 1007 is replaced by a tyrosine residue (G1007Y). Additionally, in other embodiments, the glycine residue at position 1007 is replaced by a leucine residue (G1007L). In other embodiments, the glycine residue at position 1007 is replaced by a threonine residue (G1007T). In other embodiments, the glycine residue at position 1007 is replaced by a serine residue (G1007S).

In some embodiments, the adenosine deaminase comprises a mutation at glutamic acid¹⁰⁰⁸ of the hADAR1-D amino acid sequence, or a corresponding position in a homologous ADAR protein. In some embodiments, the glutamic acid residue at position 1008 is replaced by a polar amino acid residue having a relatively large side chain. In some embodiments, the glutamic acid residue at position 1008 is replaced by a glutamine residue (E1008Q). In some embodiments, the glutamic acid residue at position 1008 is replaced by a histidine residue (E1008H). In some embodiments, the glutamic acid residue at position 1008 is replaced by an arginine residue (E1008R). In some embodiments, the glutamic acid residue at position 1008 is replaced by a lysine residue (E1008K). In some embodiments, the glutamic acid residue at position 1008 is replaced by a nonpolar or small polar amino acid residue. In some embodiments, the glutamic acid residue at position 1008 is replaced by a phenylalanine residue (E1008F). In some embodiments, the glutamic acid residue at position 1008 is replaced by a tryptophan residue (E1008W). In some embodiments, the glutamic acid residue at position 1008 is replaced by a glycine residue (E1008G). In some embodiments, the glutamic acid residue at position 1008 is replaced by an isoleucine residue (E1008I). In some embodiments, the glutamic acid residue at position 1008 is replaced by a valine residue (E1008V). In some embodiments, the glutamic acid residue at position 1008 is replaced by a proline residue (E1008P). In some embodiments, the glutamic acid residue at position 1008 is replaced by a serine residue (E1008S). In other embodiments, the glutamic acid residue at position 1008 is replaced by an asparagine residue (E1008N). In other embodiments, the glutamic acid residue at position 1008 is replaced by an alanine residue (E1008A). In other embodiments, the glutamic acid residue at position 1008 is replaced by a Methionine residue (E1008M). In some embodiments, the glutamic acid residue at position 1008 is replaced by a leucine residue (E1008L).

In some embodiments, to improve editing efficiency, the adenosine deaminase may comprise one or more of the mutations: E1007S, E1007A, E1007V, E1008Q, E1008R, E1008H, E1008M, E1008N, E1008K, based on amino acid sequence positions of hADAR1-D, and mutations in a homologous ADAR protein corresponding to the above.

In some embodiments, to reduce editing efficiency, the adenosine deaminase may comprise one or more of the mutations: E1007R, E1007K, E1007Y, E1007L, E1007T, E1008G, E1008I, E1008P, E1008V, E1008F, E1008W, E1008S, E1008N, E1008K, based on amino acid sequence positions of hADAR1-D, and mutations in a homologous ADAR protein corresponding to the above.

In some embodiments, the substrate editing preference, efficiency and/or selectivity of an adenosine deaminase is affected by amino acid residues near or in the active center of the enzyme. In some embodiments, the adenosine deaminase comprises a mutation at the glutamic acid 1008 position in hADAR1-D sequence, or a corresponding position in a homologous ADAR protein. In some embodiments, the mutation is E1008R, or a corresponding mutation in a homologous ADAR protein. In some embodiments, the E1008R mutant has an increased editing efficiency for target adenosine residue that has a mismatched G residue on the opposite strand.

In some embodiments, the adenosine deaminase protein further comprises or is connected to one or more double-stranded RNA (dsRNA) binding motifs (dsRBMs) or domains (dsRBDs) for recognizing and binding to double-stranded nucleic acid substrates. In some embodiments, the interaction between the adenosine deaminase and the double-stranded substrate is mediated by one or more additional protein factor(s), including a CRISPR/CAS protein factor. In some embodiments, the interaction between the adenosine deaminase and the double-stranded substrate is further mediated by one or more nucleic acid component(s), including a guide RNA.

According to the present invention, the substrate of the adenosine deaminase is an RNA/DNA heteroduplex formed upon binding of the guide molecule to its DNA target which then forms the CRISPR-Cas complex with the CRISPR-Cas enzyme. The RNA/DNA or DNA/RNA heteroduplex is also referred to herein as the “RNA/DNA hybrid”, “DNA/RNA hybrid” or “double-stranded substrate”. The particular features of the guide molecule and CRISPR-Cas enzyme are detailed below.

The term “editing selectivity” as used herein refers to the fraction of all sites on a double-stranded substrate that is edited by an adenosine deaminase. Without being bound by theory, it is contemplated that editing selectivity of an adenosine deaminase is affected by the double-stranded substrate's length and secondary structures, such as the presence of mismatched bases, bulges and/or internal loops.

In some embodiments, when the substrate is a perfectly base-paired duplex longer than 50 bp, the adenosine deaminase may be able to deaminate multiple adenosine residues within the duplex (e.g., 50% of all adenosine residues). In some embodiments, when the substrate is shorter than 50 bp, the editing selectivity of an adenosine deaminase is affected by the presence of a mismatch at the target adenosine site. Particularly, in some embodiments, adenosine (A) residue having a mismatched cytidine (C) residue on the opposite strand is deaminated with high efficiency. In some embodiments, adenosine (A) residue having a mismatched guanosine (G) residue on the opposite strand is skipped without editing.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)—associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided Fold Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appin cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appin cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

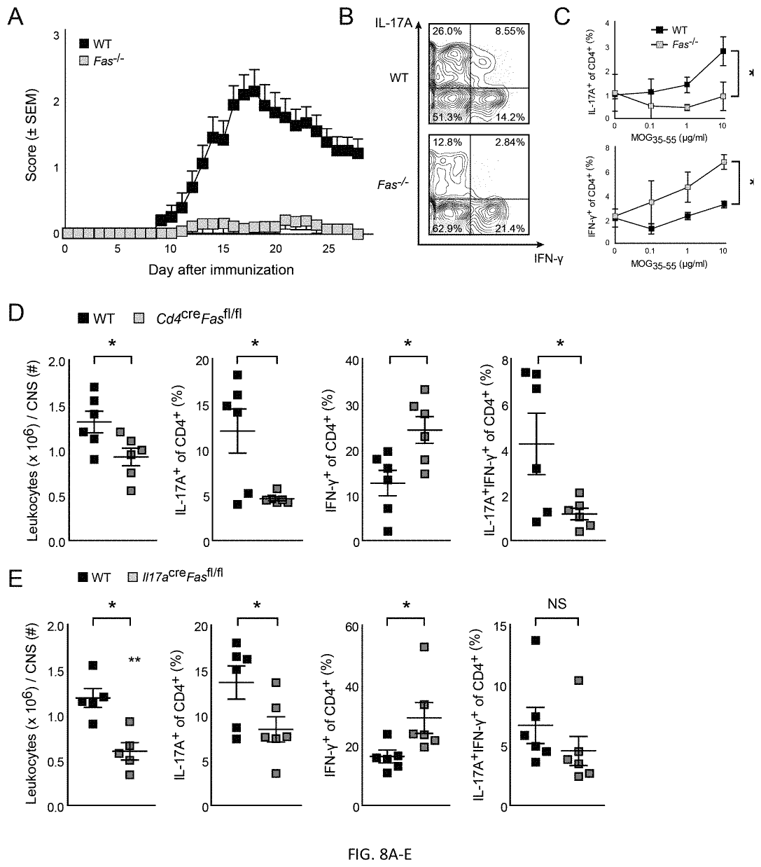

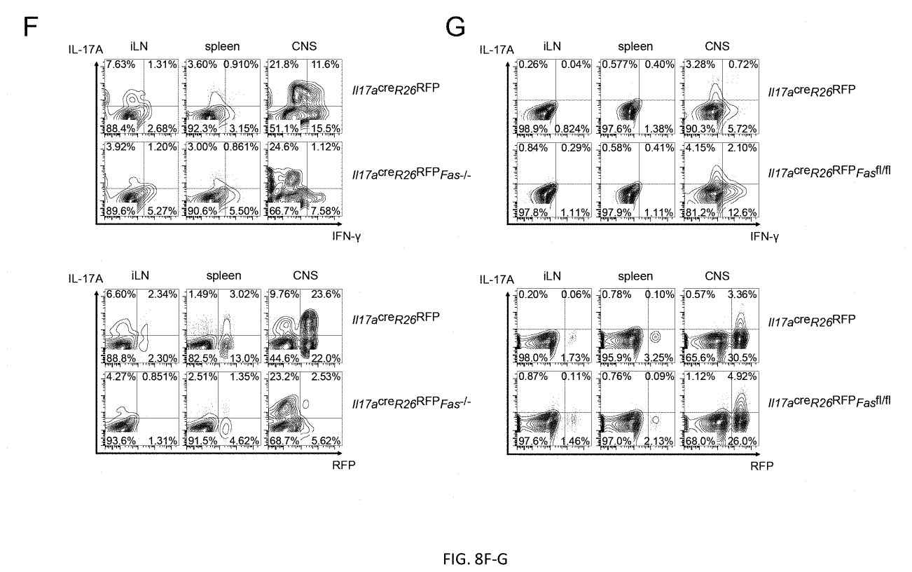

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ. I.D. No. 5)
MDPIRSRTPSPARELLSGPQPDGVQPTADRGVSP
PAGGPLDGLPARRTMSRTRLPSPPAPSPAFSADS
FSDLLRQFDPSLFNTSLFDSLPPFGAHHTEAATG
EWDEVQSGLRAADAPPPTMRVAVTAARPPRAKPA
PRRRAAQPSDASPAAQVDLRTLGYSQQQQEKIKP
KVRSTVAQHHEALVGHGFTHAHIVALSQHPAALG
TVAVKYQDMIAALPEATHEAIVGVGKQWSGARAL
EALLTVAGELRGPPLQLDTGQLLKIAKRGGVTAV
EAVHAWRNALTGAPLN

An exemplary amino acid sequence of a C-terminal capping region is:

(SEQ. I.D. No. 6)
RPALESIVAQLSRPDPALAALTNDHLVALACLG
GRPALDAVKKGLPHAPALIKRTNRRIPERTSHR
VADHAQVVRVLGFFQCHSHPAQAFDDAMTQFGM
SRHGLLQLFRRVGVTELEARSGTLPPASQRWDR
ILQASGMKRAKPSPTSTQTPDQASLHAFADSLE
RDLDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an Sin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p 65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### Binding Agents

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1gM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” (e.g., of an antibody means) that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may inhibit the ability of FAS to bind to STAT1. In certain embodiments, the blocking antibodies or antagonist antibodies or portions thereof described herein completely or partially inhibit the biological activity of the antigen(s).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., FAS and STAT1). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of proteins is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibit FAS-STAT1 interaction. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

Diseases It will be understood by the skilled person that treating as referred to herein encompasses enhancing treatment, or improving treatment efficacy. Treatment may include inhibition of an inflammatory response, tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a cellular composition, drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, in the case of cancer the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

In certain example embodiments, the pharmaceutical compositions and adoptive cell transfer strategies may be used to treat various cancers. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

In certain example embodiments, the pharmaceutical compositions and adoptive cell transfer strategies may be used to treat various autoimmune diseases. As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behcet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; S\ddot{j}ogren's syndrome; systemic lupus erythematosus; Takayasu's arteritis; temporal arteritis; vitiligo; warm autoimmune hemolytic anemia; or Wegener's granulomatosis.

Examples of inflammatory diseases or disorders include, but are not limited to, asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis.

### Administration

A “pharmaceutical composition” refers to a composition that usually contains an excipient, such as a pharmaceutically acceptable carrier that is conventional in the art and that is suitable for administration to cells or to a subject.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infusion. The skilled person will understand that compositions which are to be administered orally or topically will usually not comprise cells, although it may be envisioned for oral compositions to also comprise cells, for example when gastro-intestinal tract indications are treated. Each of the cells or active components (e.g., immunomodulants) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified immune cells and/or other active components (e.g., immunomodulants). The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

The invention is further described in the following examples, which do not limit the scope of the invention described in the claims.

### EXAMPLES

### Example 1—Fas Promotes the Differentiation and Function of Th17 Cells

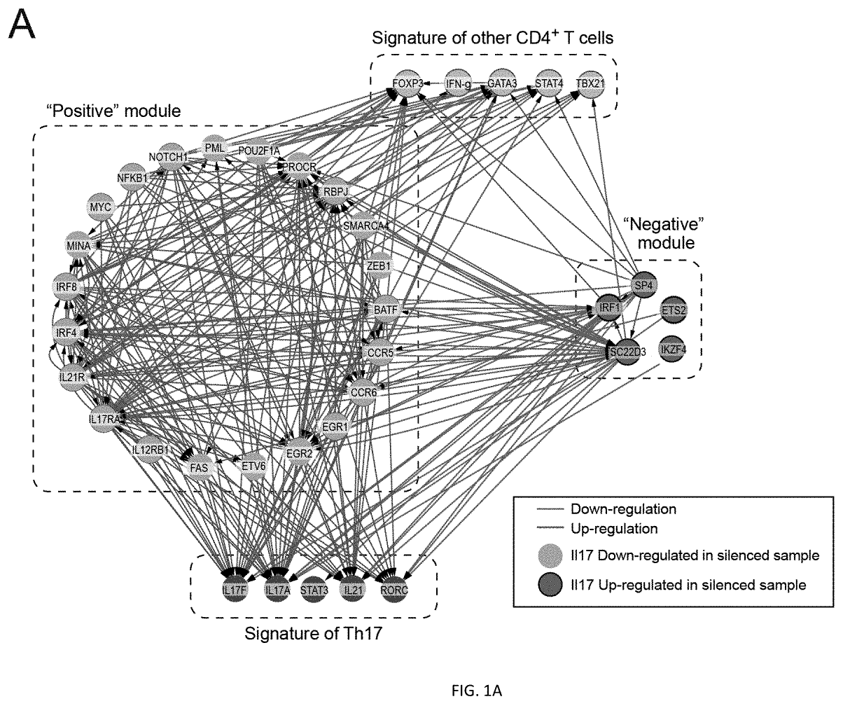

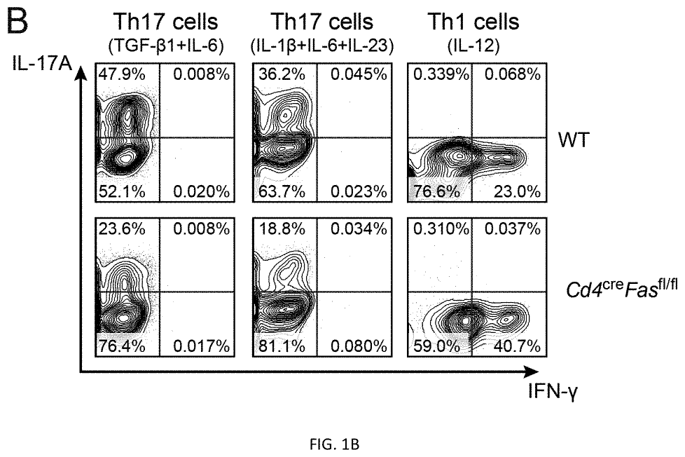

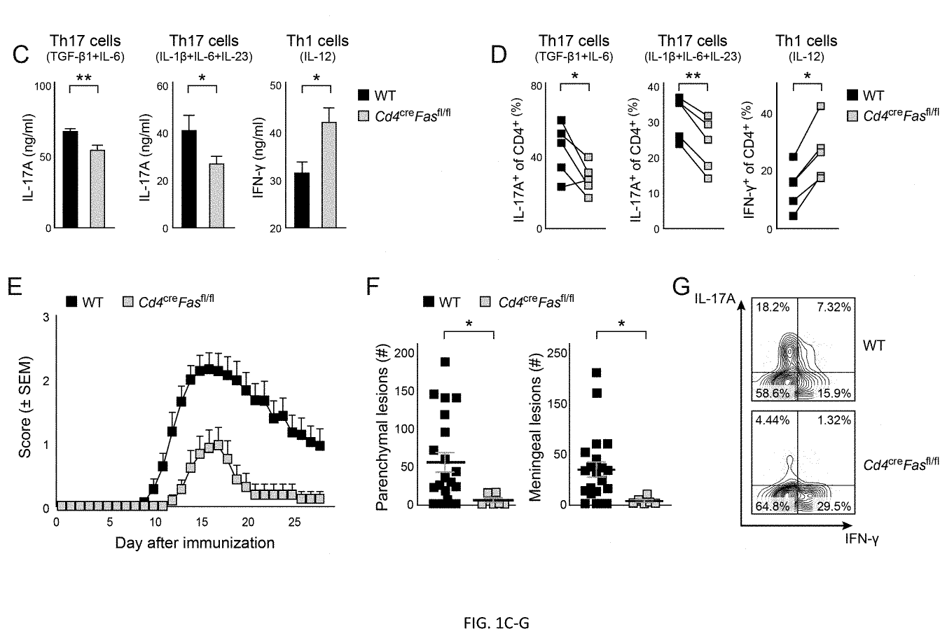

Applicants had previously generated a transcriptional network of differentiating Th17 cells that predicted Fas to be a positive regulator of the Th17 cell differentiation program (Yosef et al., 2013) and a repressor of competing T helper cell differentiation programs (FIG. 1A).

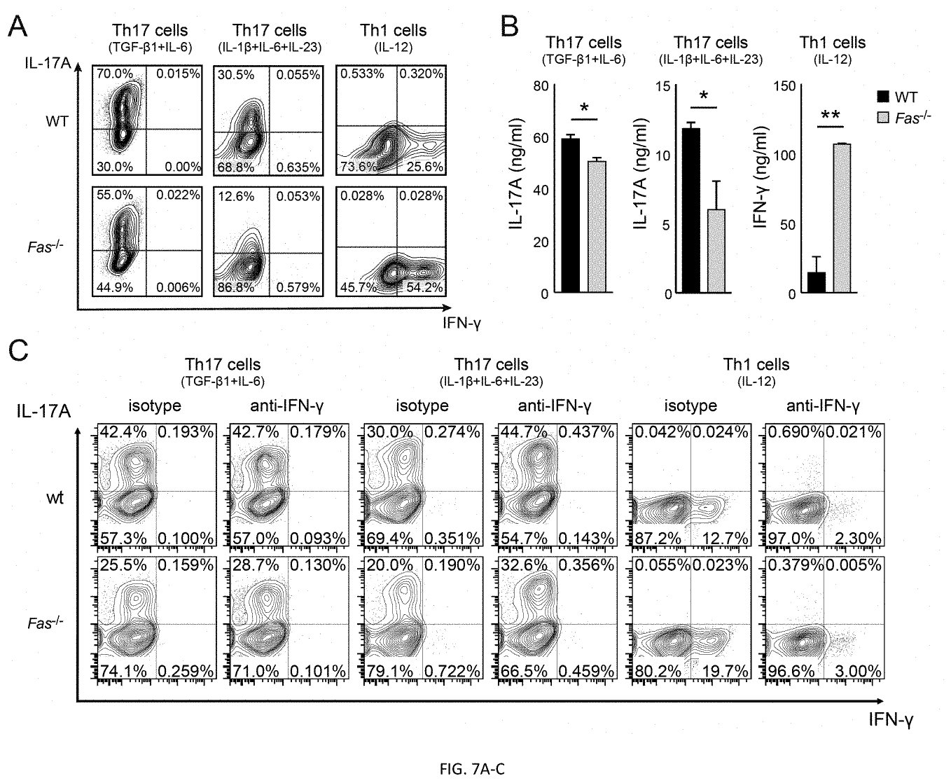

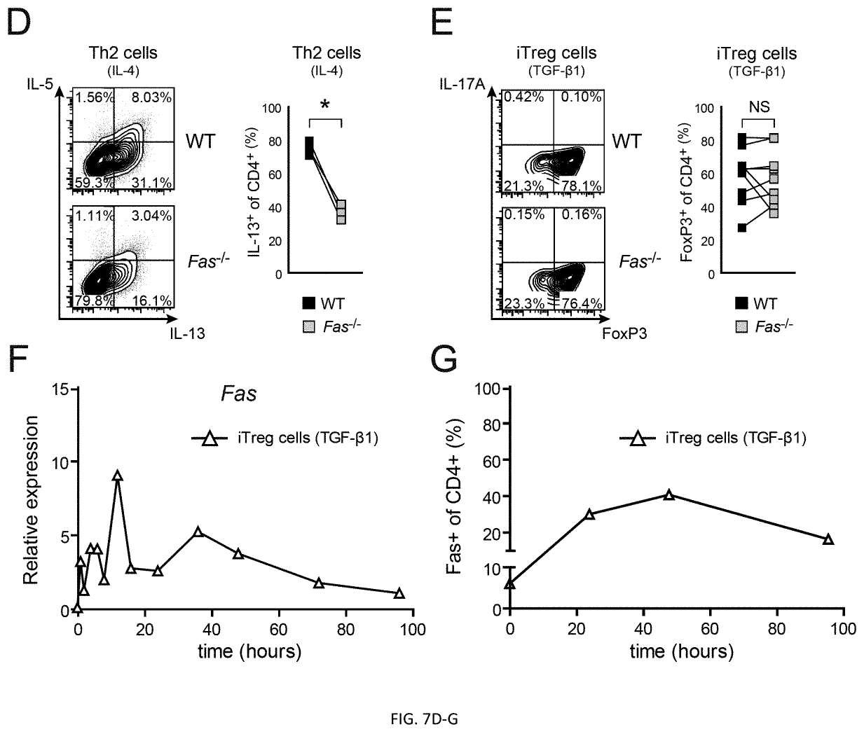

To test these predictions, Applicants first differentiated CD4⁺ T cells from wildtype (WT) and Fas-deficient mice (termed Fas^{-/-}) in vitro, using two Th17 cell polarizing conditions TGF-β1 and IL-6 (Bettelli et al., 2006b) and IL-1β, IL-6, and IL-23 (Ghoreschi et al., 2010)). Fas^{-/-} T cells produced less IL-17A under Th17 cell culture conditions, while IFN-γ production under Th1 cell culture conditions was enhanced both by intracellular cytokine staining (FIG. 7A) and by ELISA (FIG. 7B). Blocking antibodies against IFN-γ promoted Th17 cell and inhibited Th1 cell differentiation, but did not revert the difference between WT and Fas^{-/-} cells (FIG. 7C) suggesting a propensity to develop into Th1 cell over Th17 cell responses in the absence of Fas. The differentiation of Th2 cells was also impaired in Fas^{-/-} cells (FIG. 7D) while TGF-β1 induced FoxP3⁺ regulatory T (iTreg) cells were unaffected (FIG. 7E). Fas expression was downregulated in the presence of TGF-β in differentiating iTreg cells both at mRNA (FIG. 7F) and protein level (FIG. 7G). Next, Applicants induced experimental autoimmune encephalomyelitis (EAE) in the mice using myelin oligodendrocyte protein (MOG) peptide MOG₃₅₋₅₅, which requires Th17 cells for its induction (Cua et al., 2003; Lee et al., 2012). Consistent with previous studies (Waldner et al., 2004), Fas^{-/-} mice were almost completely protected from EAE (FIG. 8A). Furthermore, Fas^{-/-} mice had reduced proportions of CD4⁺ T cells producing IL-17 and co-producing IL-17 and IFN-γ and increased proportions of IFN-γ⁺CD4⁺ T cells in their central nervous system (CNS) during EAE compared to WT (FIG. 8B). The antigen-induced generation of IL-17A⁺ cells was reduced and IFN-γ⁺ T cells were increased also in draining lymph nodes in Fas^{-/-} mice in response to IL-23 stimulation (FIG. 8C).

Next, Applicants tested whether Fas-deficiency specifically affected Th cells, and for this purpose Applicants generated Cd4^{cre}Fas^{fl/fl} mice (Methods). Similar to Fas^{-/-} cells, production of IL-17A under Th17 cell differentiation conditions was reduced in Cd4^{cre}Fas^{fl/fl} cells and production of IFN-γ under Th1 cell conditions was enhanced compared to Cd4^{cre} negative littermate controls (FIG. 1B-D). Cd4^{cre}Fas^{fl/fl} mice were also strongly protected from MOG₃₅₋₅₅ induced EAE as quantified both by clinical scores (FIG. 1E) and by the number of inflammatory CNS lesions (FIG. 1F). The number of CNS-infiltrating leukocytes was lower (FIG. 8D) and CNS-infiltrating CD4⁺ T cells in Cd4^{cre}Fas^{fl/fl} mice showed a reduced frequency of IL-17 single producing and IL-17 and IFN-γ co-producing cells, while IFN-γ⁺ cells were increased (FIG. 1G and FIG. 8D). This indicates that Fas is required in T cells to promote EAE and enhances Th17 cell differentiation and represses Th1 cell-responses and thus affects multiple Th lineages.

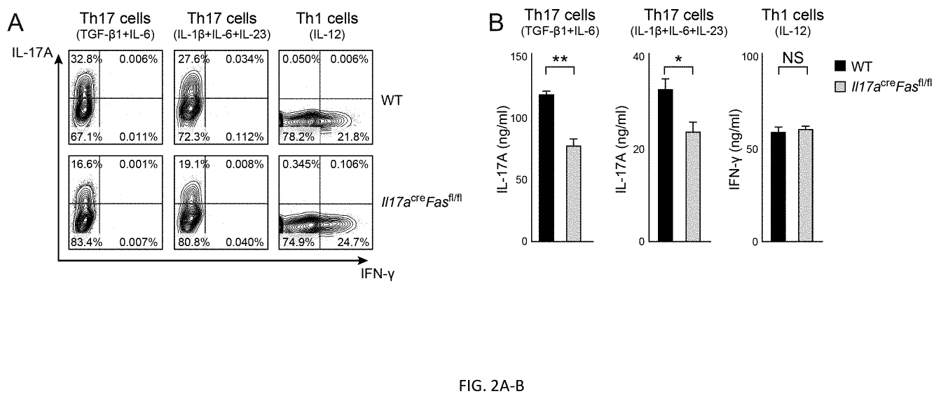

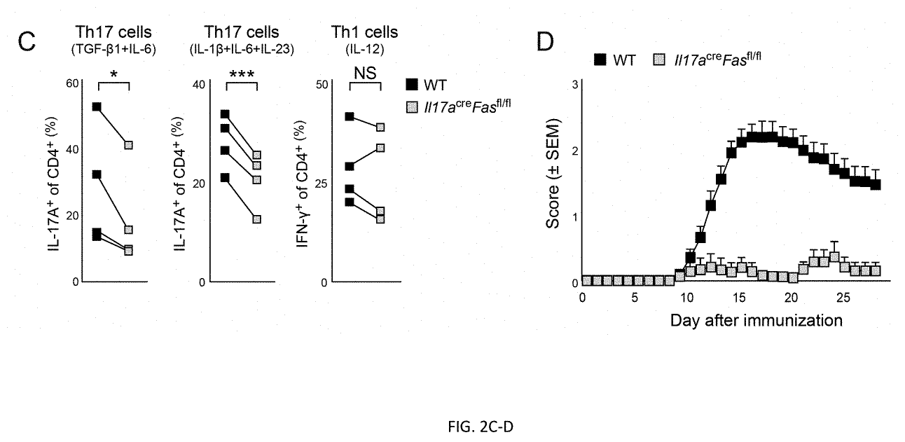

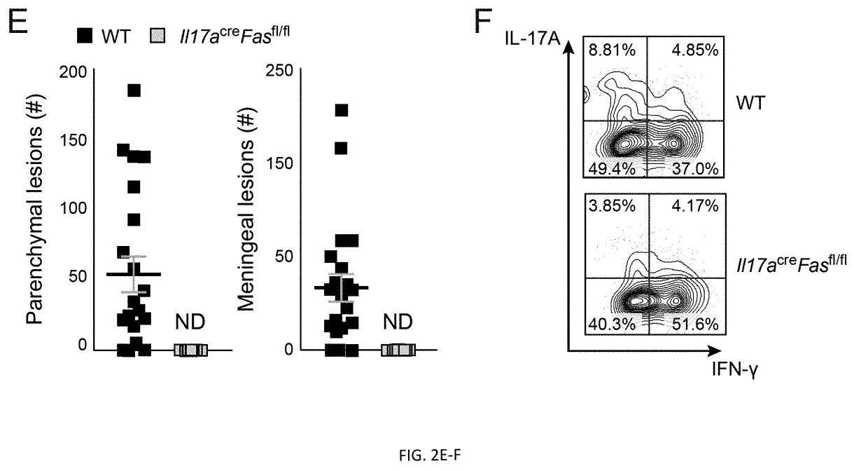

Next, Applicants tested the function of Fas specifically in Th17 cells, by generating Il17a^{cre}Fas^{fl/fl} mice, where Fas was deleted only in IL-17A producing cells (Hirota et al., 2011). T cells differentiated from these mice under Th17 cell polarizing conditions (TGF-β1, IL-6 or IL-1β, IL-6, IL-23) showed reduced IL-17A production compared to Fas-competent Il17a^{cre}Fas^{fl/wt} cells. Production of IFN-γ under Th1 cell culture conditions was unchanged in Il17a^{cre}Fas^{fl/fl} cells (FIG. 2A-C). This validated the genetic targeting because Cre expression and Fas-deletion are limited to IL-17A⁺ cells in this mouse line (Hirota et al., 2011) thus leaving Fas-expression in Th1 cells intact. In vivo, Il17a^{cre}Fas^{fl/fl} mice were almost completely protected from EAE both by clinical and histological measures (FIG. 2D, FIG. 2E). The number of CNS-infiltrating leukocytes was reduced (FIG. 8E), and CNS-infiltrating CD4⁺ T cells produced less IL-17A and more IFN-γ compared to Il17a^{cre}Fas^{fl/wt} mice (FIG. 2F and FIG. 8E). Fas is thus required specifically in Th17 cells to promote their differentiation and their in vivo function in EAE even when other lineages are Fas-competent.

### Example 2—Fas Promotes the Encephalitogenicity of Th17 Cells by Preventing Th17-to-Th1 Cell Conversion

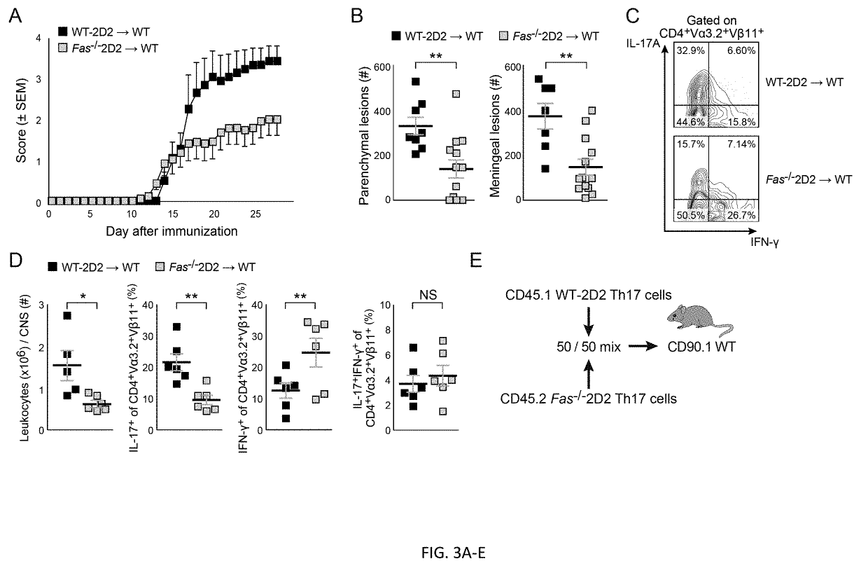

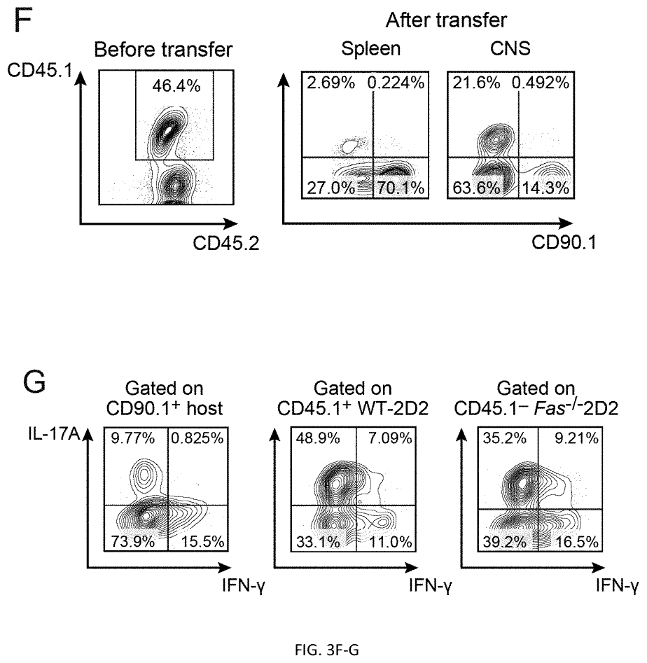

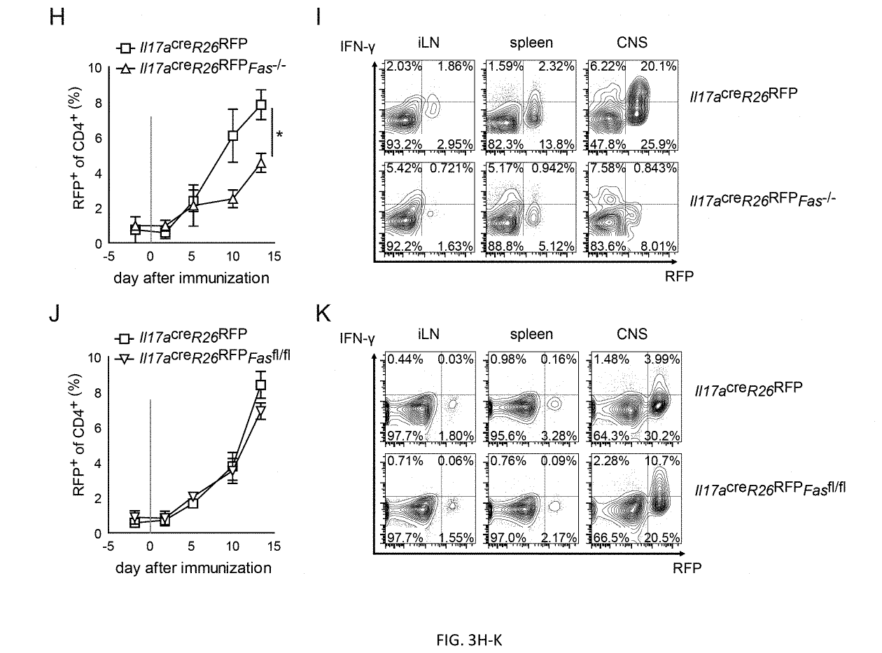

To address Th17 cell stability in vivo, Applicants next crossed Fas^{-/-} mice to 2D2 mice, which express a MOG₃₅₋₅₅-specific T cell receptor (TCR) transgene (Bettelli et al., 2003). Th17 cells differentiated in vitro from Fas^{-/-}2D2 mice induced less severe EAE compared to WT-2D2 cells after transfer into syngenic C57BL/6 WT hosts as quantified by clinical and histological measures (FIG. 3A, FIG. 3B). After transfer, CNS-infiltrating 2D2 T cells, which were identified by the Vα3.2 and Vβ11 chains of the 2D2 TCR, produced less IL-17A and more IFN-γ, while co-producers of IL-17A and IFN-γ were unchanged (FIG. 3C, FIG. 3D) despite comparable pre-transfer cytokine production (data not shown). Fewer leukocytes infiltrated the CNS after transfer of Fas^{-/-}2D2 than WT-2D2 donor cells (FIG. 3D). Together this indicates that in the absence of Fas, differentiated Th17 cells lose their encephalitogenicity in vivo with increased accumulation of Th1-like cells.

Applicants next tested for a T cell intrinsic effect of Fas-deficiency in vivo. Applicants crossed CD45.1 congenic C57BL/6 mice with 2D2 mice (Methods) to transfer EAE with defined antigen specificity (Jager et al., 2009). Applicants differentiated and transferred a 50 to 50 mixture of CD4⁺CD45.1⁺WT-2D2 and CD4⁺CD45.2⁺ Fas^{-/-}2D2 Th17 cells into CD90.1⁺ WT hosts (FIG. 3E). After transfer, the proportion of Fas^{-/-}2D2 cells (CD4⁺CD45.1⁻CD90.1⁻) was approximately 4-fold greater than WT-2D2 (CD4⁺CD45.1⁺CD90.1⁻) cells in the periphery and in the CNS of recipients developing EAE (FIG. 3F) consistent with the known enhanced homeostatic expansion of Fas-deficient T cells in vivo (Fortner and Budd, 2005). At the peak of EAE, Fas^{-/-}2D2 cells (CD4⁺CD45.1⁻CD90.1⁻) produced less IL-17A and more IFN-γ than WT-2D2 cells (CD4⁺CD45.1⁺CD90.1⁻) (FIG. 3G), indicating that the increased propensity for IFN-γ production in the absence of Fas is a cell intrinsic phenomenon. Of note, Applicants transferred CD4⁺ cells arguing against a contribution of CD4 negative TCRαβ⁺CD4⁻CD8⁻B220⁺ atypical lymphocytes in these experiments. Furthermore, Fas^{-/-}2D2 Th17 cells relatively expand more in this mixed transfer setting (FIG. 3F), although they are less pathogenic and produce less IL-17A. This argues for a reduced encephalitogenicity on a per cell basis and suggested that the influence of Fas-deficiency on apoptosis and encephalitogenicity may be independent phenomena.

To further delineate the lineage ontogeny of the IFN-γ⁺ T cells increased in Fas-deficient mice, Applicants next generated Il17a^{cre}R26^{RFP} mice crossed to Fas^{-/-} mice (Hirota et al., 2011; Meyer Zu Horste et al., 2016). Applicants found that the continuous accumulation of CD4⁺RFP⁺ cells in the periphery after EAE induction was reduced in Il17a^{cre}R26^{RFP}Fas^{-/-} mice compared to Il17a^{cre}R26^{RFP} mice (FIG. 311). Instead, Il17a^{cre}R26^{RFP}Fas^{-/-} mice showed an expansion of CD4⁺RFP⁻IFN-γ⁺ (i.e. non-Th17) cells both in the periphery and in the CNS at peak of EAE (FIG. 3I and FIG. 8F). This indicates that global Fas deficiency in Fas^{-/-} mice impairs the initial lineage commitment of Th17 cells causing more cells to deviate to a Th1 cell phenotype before RFP expression and Th17 cell differentiation.

Applicants next generated Il17a^{cre}R26^{RFP}Fas^{fl/fl} mice to lineage trace Th17 cells while at the same time restricting Fas deletion only to IL-17A⁺ cells. The generation of CD4⁺RFP⁺ cells in the periphery after EAE induction was unchanged in Il17a^{cre}R26^{RFP}Fas^{fl/fl} mice (FIG. 3J). However, IFN-γ⁺ cells in Il17a^{cre}R26^{RFP}Fas^{fl/fl} mice were increased at peak of EAE and almost all IFN-γ⁺ cells were also RFP⁺ and IFN-γ⁺RFP⁺ cells were increased 2-2.5 fold in Il17a^{cre}R26^{RFP}Fas^{fl/fl} mice (i.e. IFN-γ⁺Th17 cells) compared to Il17a^{cre}R26^{RFP} mice (FIG. 3K and FIG. 8G) while IL-17A⁺RFP⁺ cells were unchanged (FIG. 8G). Th17 cell-restricted Fas deficiency thus causes instability of Th17 cells and their deviation towards a Th1 cell-like phenotype after successful initial commitment to the Th17 cell lineage. This indicates that Fas is persistently required to maintain the Th17 cell differentiation program in vivo.

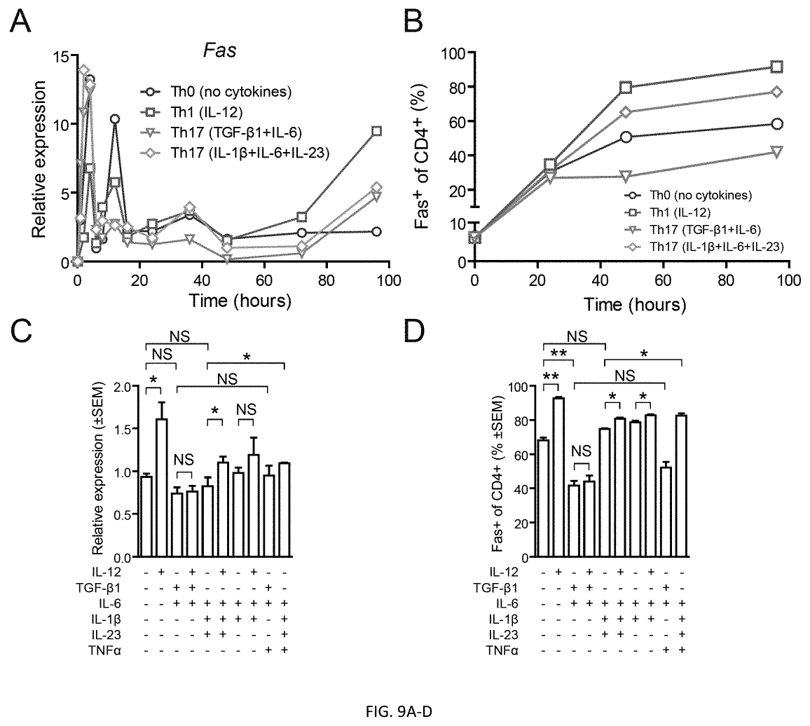

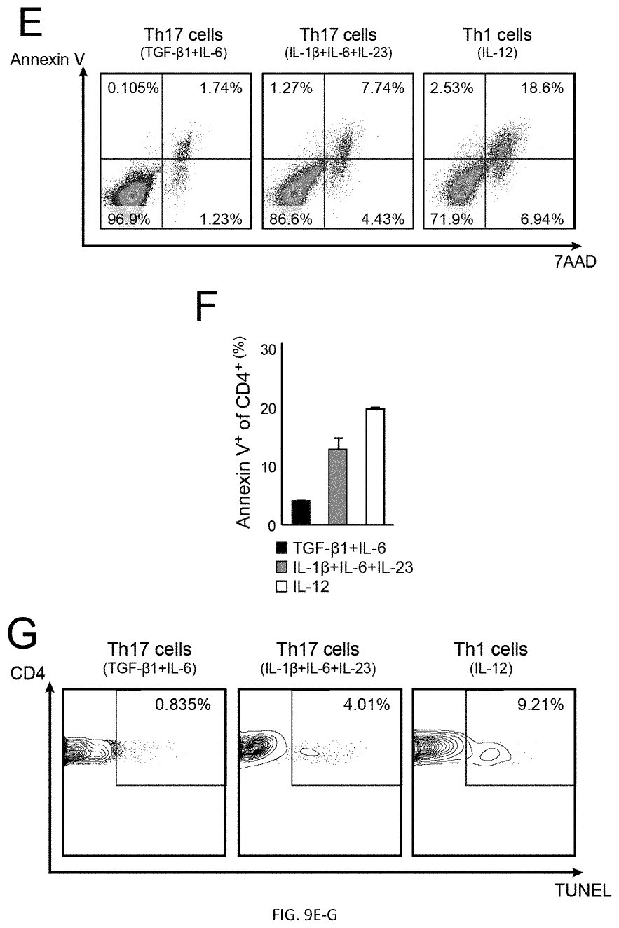

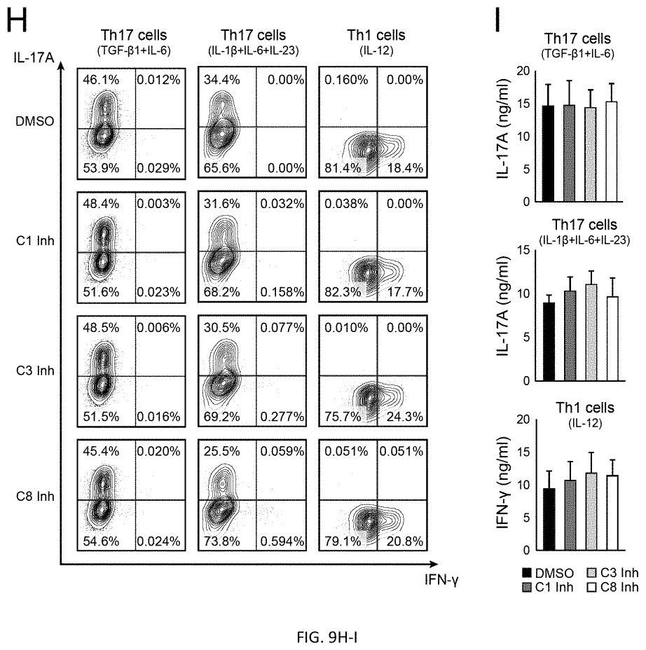

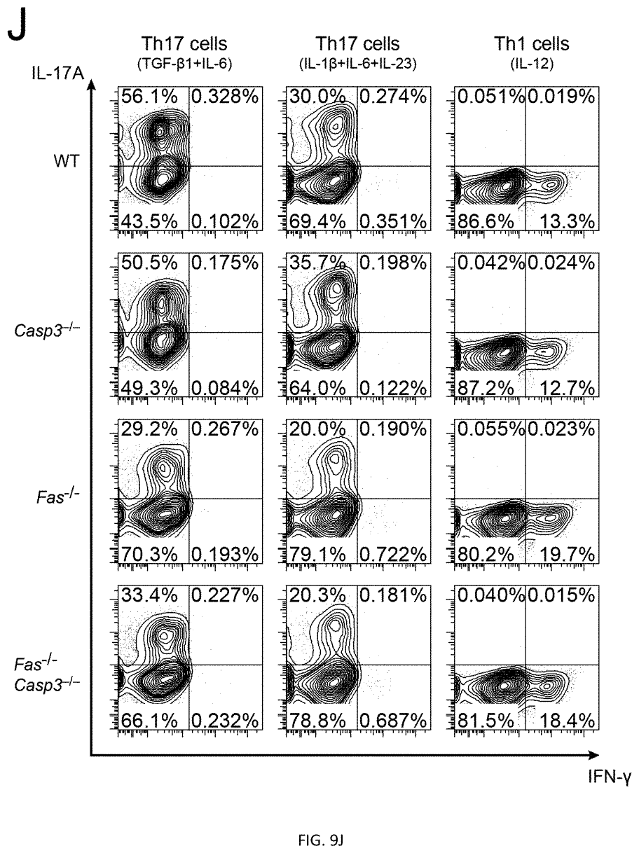

Applicants next tested how Fas expression is regulated during T cell differentiation beyond its known induction by TCR engagement and T cell activation (Zheng et al., 2001). In all Th cell subsets, Fas mRNA expression exhibited two early peaks at 4 and 12 hours that were highest in Th0 cells and Fas was then continuously upregulated between 48 and 96 hours with final expression being highest under Th1 cell and lower under Th0 cell and Th17 cell conditions (FIG. 9A). Cell surface Fas protein amounts increased in all subsets with culture duration and were highest in Th1 cell and lowest under TGF-β with IL-6 induced Th17 cell conditions (FIG. 9B). Applicants next tested whether Th1 cell-inducing IL-12 controlled Fas expression in Th17 cells. Applicants found that IL-12 (and TNFα) enhanced Fas expression on the mRNA and protein level under IL-1β, IL-6, IL-23 conditions, but not under TGF-β1 with IL-6 conditions (FIG. 9C, FIG. 9D). IFN-γ and IL-4 had no effect on Fas mRNA expression (data not shown). In accordance with its role in activation induced cell death, Fas is thus upregulated by activation in all Th cell lineages and is strongly upregulated by Th1 cell inducing cytokines. Induction of Fas by IL-12 may form an inhibitory feed back loop to limit excessive Th1 cell differentiation.

### Example 3—Caspase-Inhibition or -Deficiency do not Replicate the Fas-Deficiency Phenotype

To address the role of apoptosis in the Th17 cell phenotype in Fas-deficient cells, Applicants measured hallmarks of apoptosis during in vitro T cell differentiation. Apoptosis rates were overall lower under Th17 cell culture conditions than under Th1 cell conditions, and lower in Th17 cells differentiated with TGF-β1 and IL-6 than in Th17 cells generated with IL-1β, IL-6, IL-23 (FIG. 9E, FIG. 9G). A higher propensity of Th1 cells than Th17 cells for Fas-dependent apoptosis has been described previously (Cencioni et al., 2015), but did not explain the reduced lineage stability triggered by Th17-cell restricted Fas deletion. Next, Applicants blocked Caspase activation during T cell differentiation. Applicants did not observe differences in the production of IL-17A or IFN-γ under Th17 cell or Th1 cell culture conditions in the presence of Caspase-1, -3, or -8 inhibitors (FIG. 9H,J), or in Caspase-3 deficient (Casp3^{-/-}) or Fas^{-/-}Casp3^{-/-} double-deficient cells compared to WT cells (FIG. 9J). Thus, inhibiting the canonical death receptor pathway thus does not phenocopy Fas-deficiency, suggesting that apoptosis-independent mechanisms underlie the Th17-to-Th1 cell conversion in the absence of Fas.

### Example 4—A STAT1 Dependent Program is Activated in Fas-Deficient Th17 Cells

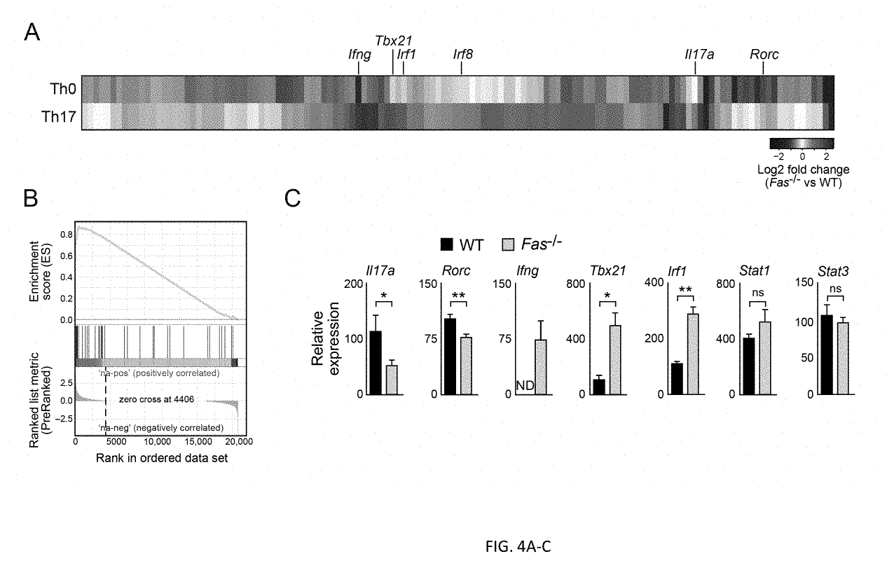

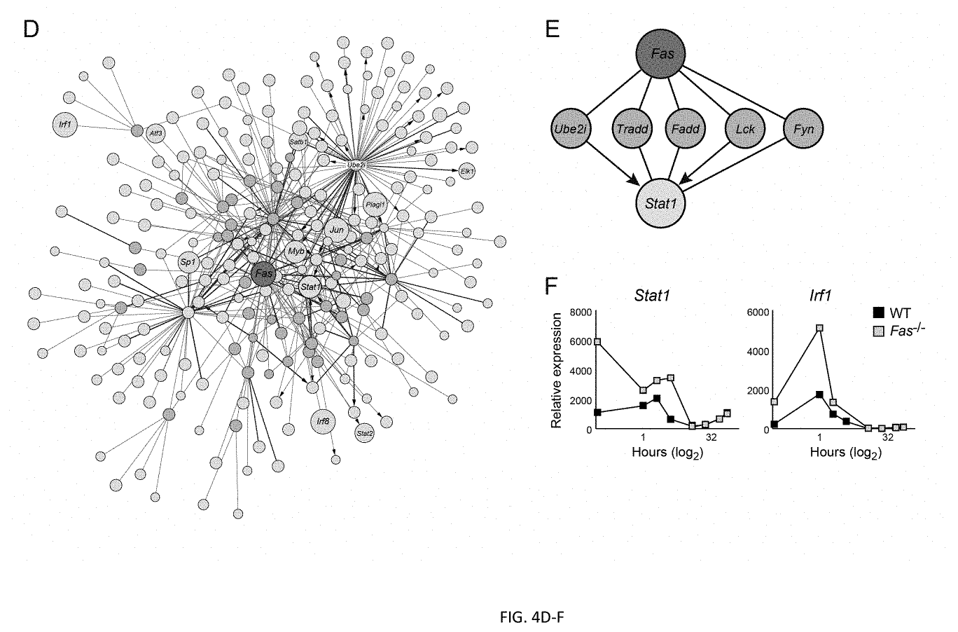

To identify potential alternative mechanisms by which Fas controls Th17 cell differentiation and stability, Applicants performed RNA-sequencing (Methods). A signature of key Th17 cell-related transcripts (e.g., Il17f, Il6ra, Rorc) was significantly downregulated in Fas-deficient Th17 cells (Cuffdiff p-values 9.5×10⁻⁴, 2.1×10⁻², 4.8×10⁻², test described in (Trapnell et al., 2010)), including the Th17 cell signature cytokine Il17a (Cuffdiff p-value_ENREF_41<5×10⁻⁵, False Discovery Rate (FDR)=0.01; FIG. 4A), whereas key Th1 cell-related transcripts and transcripts controlled by IFN signalling were up-regulated (e.g., Irf1, Irf8, Tbx21, Gbp2, Gbp4, Gbp5, Tap1; FIG. 4A). Notably, a signature of genes related to IFN-γ signalling was upregulated in Fas^{-/-} cells under both Th17 cell conditions (Rank 1 (FDR=0.051)) (FIG. 4B) and Th0 cell conditions (Rank 3 (FDR=0.066)) (data not shown) in gene set enrichment analysis (GSEA) (Methods). This suggested that enhanced IFN-signalling is involved in the Th1 cell-like transcriptional profile of Fas-deficient CD4⁺ T cells even under non-polarizing conditions. Testing key transcripts by qPCR confirmed that Il17a and Rorc were significantly reduced and Ifng, Tbx21, and Irf1 increased in Fas-deficient Th17 cells (FIG. 4C).

To find transcription factors (TFs) that were most likely responsible for the Fas-deficiency phenotype, Applicants performed an integrative network analysis (Methods). Briefly, the analysis aimed to find potential paths from the perturbed receptor (Fas) through protein-protein interaction (PPI) to TFs controlling differentially expressed genes, by integrating PPI, TF motif binding, and the RNA-seq expression data.

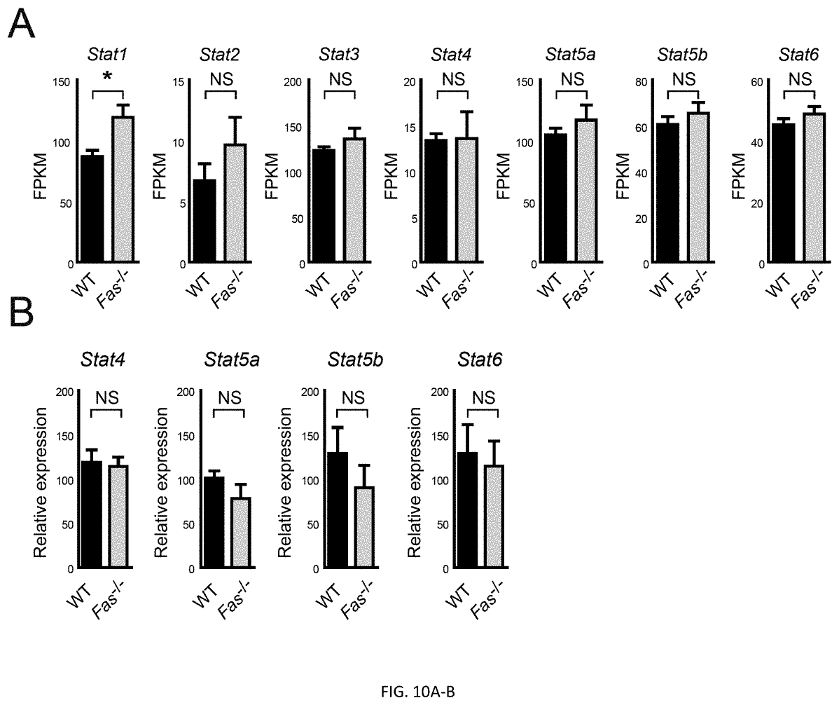

Applicants found Stat1 to have the strongest integrated evidence as both being regulated by Fas and being the cause of the observed phenotype (bootstrap p-value <10′) (FIG. 4D). Additional analyses corroborated this prediction. First, one of the Stat1 DNA-binding motifs was among the eight TF promoter binding motifs that were enriched in the promoters of differentially expressed genes (Irf1, Sp1, Atf3, Irf7, Irf8, Stat1, Elk1, Irf2) (Suppl. Tab. 1). Second, Stat1 also contributed to the enrichment score of the interferon-γ-signalling set in GSEA (FIG. 4B), along with the TFs Irf1, Irf8, and Sp100. Third, querying the regulatory modules from the Ontogenet model of mouse hematopoiesis in the Immunological Genome Project (Methods) implicated Dtx31, and Stat1 as regulators of two of the most significantly enriched expression modules in the dataset (FDR<0.05). Two of the network paths between Fas and STAT1 in the model (FIG. 4E) passed through TRADD and FADD, which physically interact with STAT1 (Wang et al., 2000), providing a potential mechanism for Fas-dependent regulation of STAT1. Stat1 itself was not differentially expressed in the RNA-seq dataset obtained at 48 hours, but signalling through STAT-proteins occurs rapidly. In a time-course expression analysis Stat1 was indeed upregulated in naïve Fas^{-/-} cells between 0 and 4 hours under Th17 cell differentiation conditions (FIG. 4F), which may reflect ongoing self-induction of STAT1 in naïve Fas^{-/-} cells before sorting. Irf1, which is part of the Th1 cell-transcriptional program and is transcriptionally controlled by STAT1 (Kano et al., 2008), was also upregulated in Fas^{-/-} Th17 cells and its peak expression followed Stat1 expression (FIG. 4F). Up-regulation of STAT1 thus precedes Irf1, Tbx21, and Ifng, consistent with STAT1 being the most proximal TF controlled by Fas and controlling the IFN-signalling signature in the dataset. Other members of the STAT-protein family were not differentially expressed in the RNA-seq dataset (FIG. 10A) or by qPCR (FIG. 10B) suggesting a Stat1-specific effect of Fas-deficiency.

### Example 5—Fas Binds STAT1 and Reduces its Phosphorylation and Nuclear Translocation

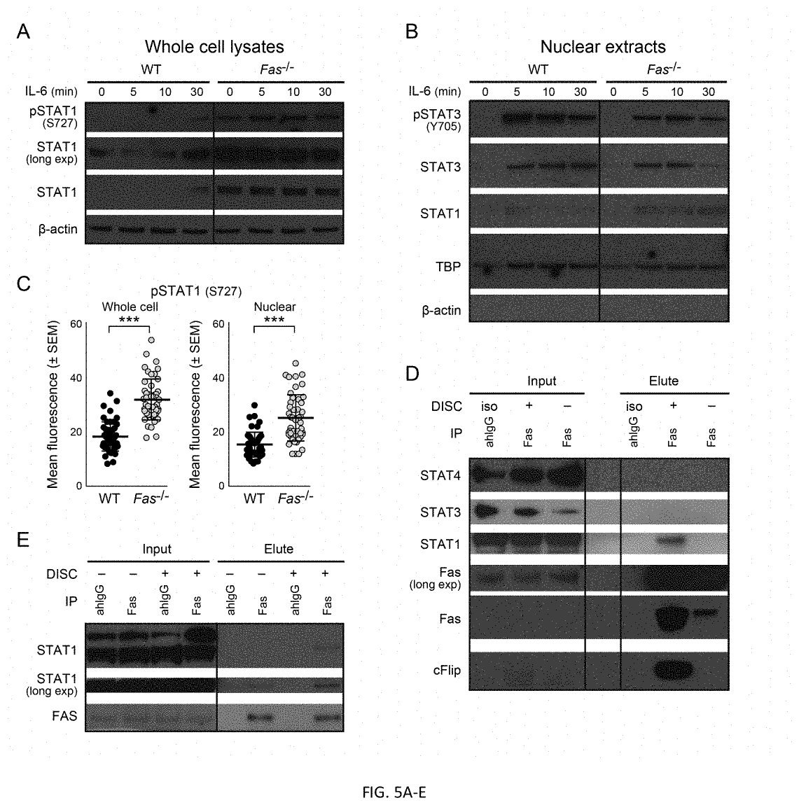

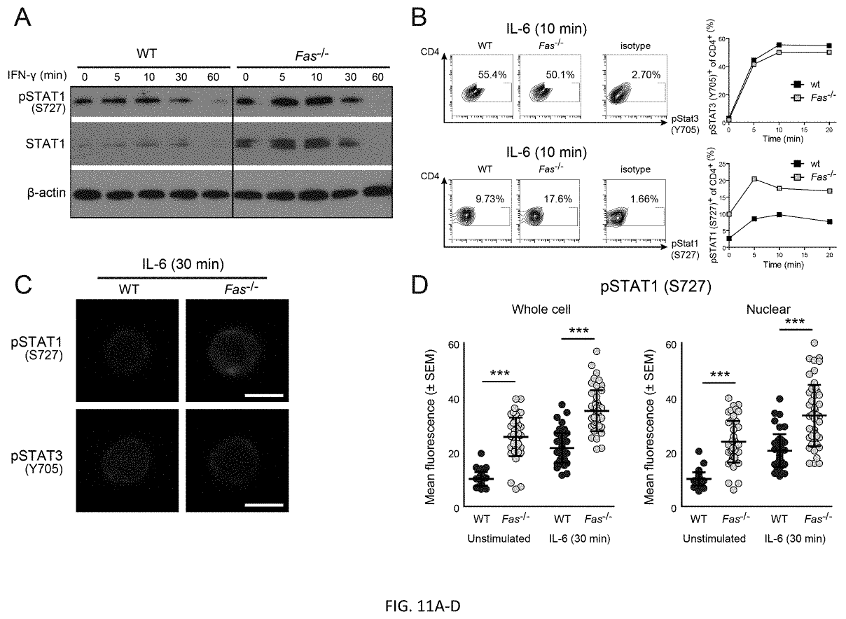

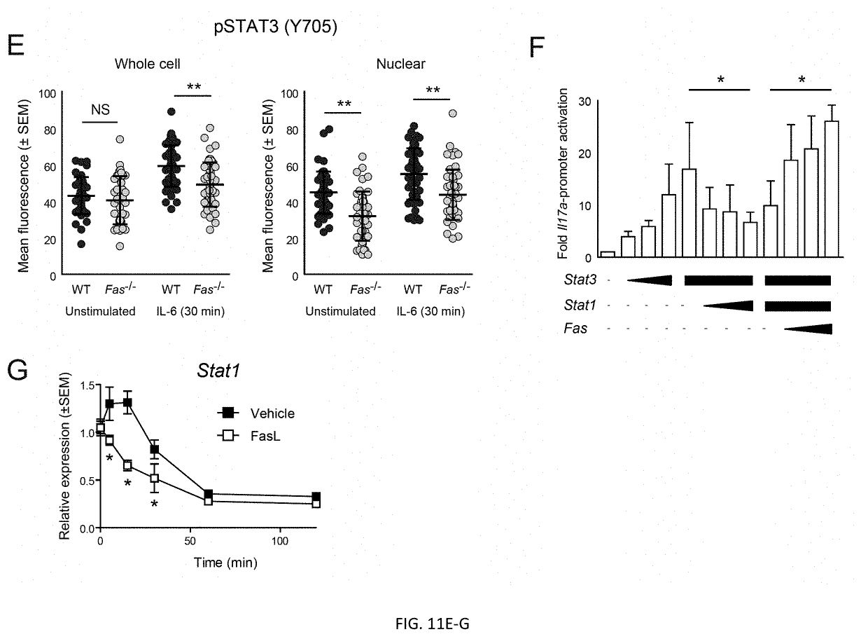

Applicants next tested how Fas impacted the expression and phosphorylation of the STAT1 protein. Fas-deficient naïve CD4⁺ T cells contained higher amounts of both total STAT1 and pSTAT1 under baseline conditions and upon activation by IFN-γ (FIG. 11A). The increased total STAT1 protein amounts may reflect ongoing self-induction of STAT1 transcription even in naïve cells. Th17 cell differentiation does not usually involve IFN-γ dependent signals, but essentially depends on IL-6 activating STAT3 (Harris et al., 2007). The amounts of both total STAT1 and pSTAT1 at baseline and following IL-6 stimulation were higher in Fas-deficient cells compared to WT cells by Western blotting (FIG. 5A) and by flow cytometry (FIG. 11B). STAT1 activity depends on translocation of pSTAT1 to the nucleus. Applicants found that STAT1 nuclear translocation was enhanced in response to IL-6 stimulation in Fas^{-/-} CD4⁺ T cells compared to WT both in nuclear extracts (FIG. 5B) and by cell imaging (FIG. 5C and FIG. 11C, FIG. 11D). The absence of β-actin signal in FIG. 5B supports that the nuclear fractions were not contaminated by cytoplasmic contents. IL-6 induces and phosphorylates STAT3 which induces Th17 cell differentiation (Harris et al., 2007; Yang et al., 2007). Notably, STAT1 is known to heterodimerize with STAT3 and inhibit STAT3-dependent transcription (Hu and Ivashkiv, 2009; Villarino et al., 2017). Indeed, the nuclear translocation of STAT3 and pSTAT3 was concomitantly reduced after IL-6 stimulation in Fas^{-/-} T cells compared to WT (FIG. 5B and FIG. 11C, FIG. 11E). Thus, Fas deficiency triggers an excessive activation of STAT1 in response to the Th17 cell-inducing cytokine IL-6, which in turn may inhibit the activation of STAT3 and the expression of STAT3-dependent transcripts (e.g. Il17a, Rorc), while promoting the expression of STAT1-dependent transcripts. In accordance, Applicants found that STAT1 overexpression inhibited the STAT3-driven activation of the Il17a promoter and that this STAT1-dependent cross-inhibition of a known STAT3 target gene was partially reverted by overexpression of Fas in promoter activation studies (FIG. 11F). Fas thus controls the balance between two opposing STAT-proteins.

Next, Applicants tested the protein-protein interaction predicted in the network model between Fas and STAT1 (FIG. 4E). In a mouse T cell line, STAT1 co-immunoprecipitated with Fas after cross-linking of the DISC (FIG. 5D), while neither STAT3 nor STAT4 co-immunoprecipitated with Fas (FIG. 5D). STAT1 also physically interacted with Fas in primary mouse CD4 cells and this was enhanced by DISC assembly (FIG. 5E). Thus, STAT1, but not STAT3 or STAT4, physically interacts with Fas, and this binding is further enhanced by binding of Fas to its ligand. Consistent with this observation, Applicants also found that binding of Fas to its ligand, repressed Stat1 expression early during Th cell activation (FIG. 11G). Taken together, the findings support the idea that Fas binds and inhibits STAT1 activation and nuclear translocation (FIG. 5A-5C) by sequestering STAT1 at the cell membrane and thus indirectly promotes STAT3 dependent transcription required for Th17 cell differentiation.

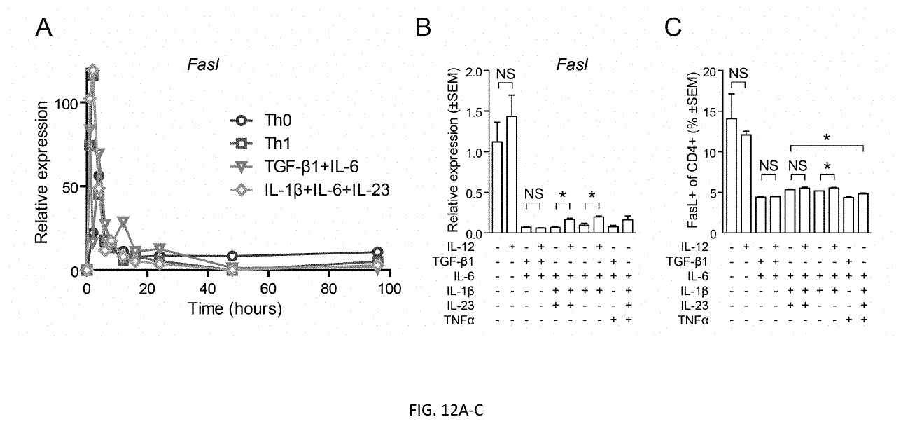

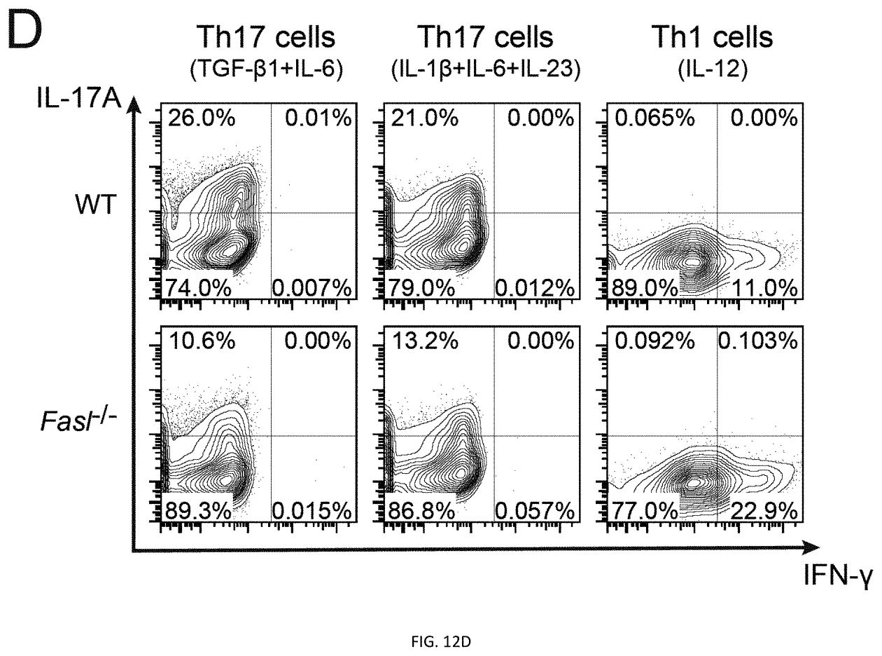

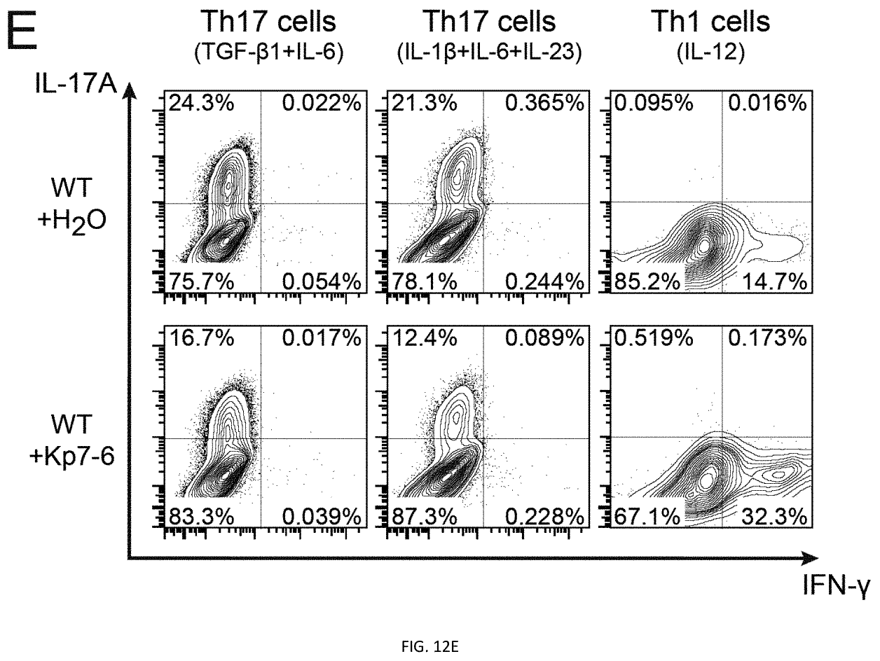

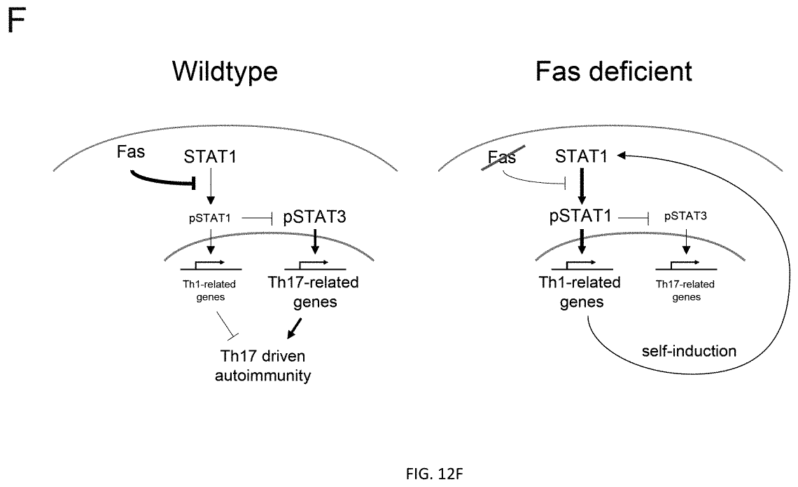

Next, Applicants studied the source and requirement of the Fas ligand (FasL) signal in vitro. On mRNA level, Fasl expression showed an early peak at 1-4 hours under all differentiation conditions and was subsequently downregulated but expressed in all subsets (FIG. 12A). After 96 hours of culture, Th0 and Th1 cell-differentiated cultures expressed most Fasl while Th17 cell-inducing cytokines repressed FasL expression quantified both by mRNA (FIG. 12B) and protein (FIG. 12C) measurements. Fasl expression was higher in Th1 cells than in Th17 cells and IL-12 was unable to induce FasL expression in Th17 cells (FIG. 12B, FIG. 12C). Similar to Fas^{-/-} cells, Fasl^{-/-} cells showed an increase of IFN-γ and a decrease of IL-17A production under Th1 cell and Th17 cell culture conditions, respectively (FIG. 12D). Addition of the small molecule compound Kp7-6, that blocks FasL with Fas interaction, also enhanced Th1 cell and reduced Th17 cell differentiation (FIG. 12E). Overall, these findings suggest that the decrease in Th17 cell differentiation and increase in Th1 cell phenotype in Fas-deficient mice depends on Fas with FasL interaction and that the ligand is present at early stages of T helper cell differentiation.

### Example 6—Stat1 Co-Deficiency Rescues the Fas-Deficiency Mediated Effect on Th17 Cell Differentiation

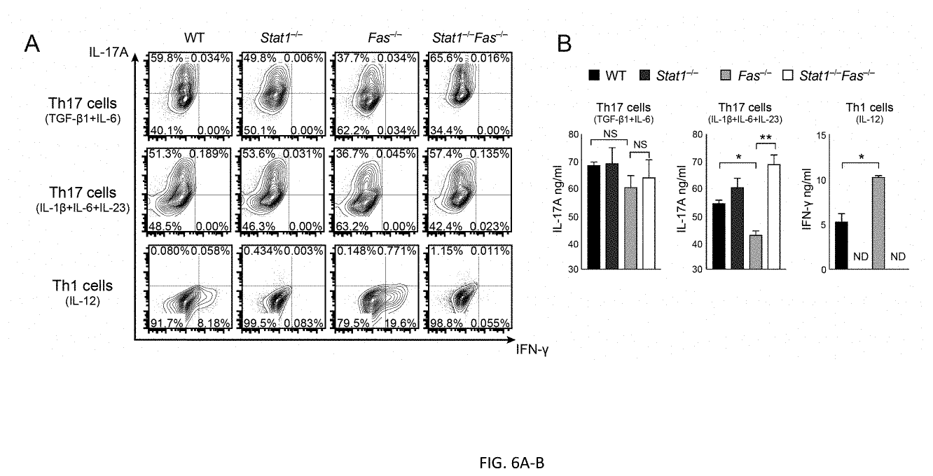

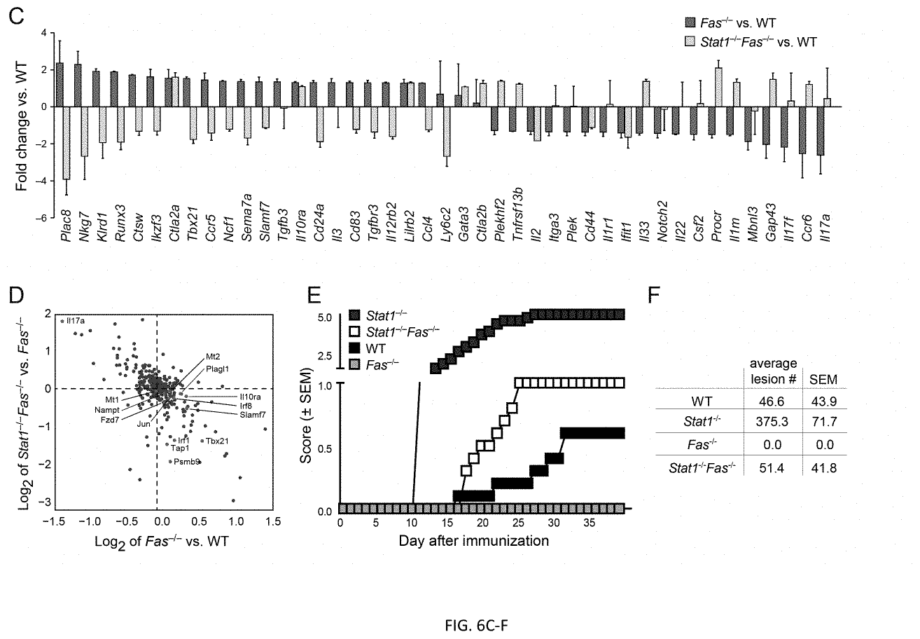

To directly test the role of STAT1 in Fas-deficient T cells, Applicants generated mice double-deficient for Stat1 and Fas. As previously described (Peters et al., 2015), Th17 cell differentiation was unaffected by Stat1 deficiency (FIG. 6A, FIG. 6B). The defect in IL-17A production caused by Fas-deficiency was fully rescued in Stat1^{-/-}Fas^{-/-} cells, under both Th17 cell differentiation culture conditions, although the effect was more pronounced under IL-1β, IL-6, IL-23 than TGF-β1 with IL-6 conditions (FIG. 6A, FIG. 6B). Consistent with the known role of STAT1 in Th1 cell differentiation (Afkarian et al., 2002), IFN-γ production under Th1 cell differentiation conditions was abolished in Stat1^{-/-} and Stat1^{-/-}Fas^{-/-} cells (FIG. 6A, FIG. 6B).

Applicants next analyzed how Stat1-deficiency impacted the gene expression defect observed in Fas^{-/-} Th17 cells. Analyzing a pre-defined 250 gene signature of Th17 cell-related transcripts (Yosef et al., 2013) showed that Fas/STAT1 double-deficiency reverted many of the expression changes induced by Fas deficiency under Th17 cell culture conditions. Applicants found that the reduced expression of Il17a, IL17f and increased expression of Tbx21, Irf1 in Fas-single deficient cells were all rescued in Stat1^{-/-}Fas^{-/-} cells (FIG. 6C, FIG. 6D). Of note, Ifng overexpression was also rescued, but did not reach the expression cut-off for detection in Th17 cells.

Finally, Applicants tested the role of STAT1 in the protection from EAE in Fas-deficient mice. To specifically focus on STAT1 in CD4 cells and circumvent the known susceptibility of global Stat1^{-/-} mice to viral infections, Applicants reconstituted Rag1^{-/-} mice with purified CD4⁺ T cells with wild type or Fas-deficient CD4 T cells. While mice reconstituted with Fas-deficient T cells were protected from EAE, the recipients of Stat1^{-/-}Fas^{-/-} cells developed clinical signs of EAE (FIG. 6E) and histologically detectable CNS lesions (FIG. 6F). In line with the exacerbated EAE developing in Stat1^{-/-} mice (Bettelli et al., 2004) recipients of Stat1^{-/-} cells developed severe EAE (FIG. 6E), a high number of histological lesions (FIG. 6F) and had to be sacrificed early. The defect in Th17 cell differentiation in Fas-deficient T cells thus dominantly depends on the excessive presence of STAT1 and can be rescued by Stat1 deletion in vivo. This identifies a mechanistic model whereby Fas controls the Th17-to-Th1 cell balance and autoimmunity through STAT1.

### Example 7—Discussion

In this study Applicants demonstrated that Fas-deficiency impaired Th17 cell differentiation and grossly attenuated EAE accompanied by a shift towards enhanced Th1 cell generation. This effect appeared to be apoptosis-independent. Computational modelling identified a Th1 cell-like gene expression signature in Fas-deficient Th17 cells and nominated STAT1 as a top inferred transcriptional regulator of this signature. Indeed, Applicants showed that Fas physically interacted with STAT1 and loss of Fas increased STAT1 activation, while dual deficiency of STAT1 and Fas rescued the Th17 defect. Consistent with these in vitro data, EAE susceptibility was restored in mice repleted with Fas-STAT1 double-deficient CD4⁺ T cells. The findings thus emphasize an apoptosis-independent function of Fas protein, whereby Fas regulated the balance between the antagonizing STAT1 and STAT3 proteins and competing differentiation programs in different T helper subsets, and consequently organ specific autoimmunity.

The link Applicants established here between Fas- and STAT1-signalling provides a mechanistic explanation for the long-standing conundrum that Fas-deficient mice are almost completely protected from EAE (Waldner et al., 1997). The excessive STAT1 activation in Fas-deficient cells impaired the differentiation and stability of Th17 cells, that are an essential driver of EAE (Cua et al., 2003; Lee et al., 2012). The observations suggest a dual effect of Fas on STAT1: First, naïve Fas-deficient cells expressed more STAT1 protein indicating that Fas continuously repressed STAT1 activation and self-induction in naïve T cells. Second, cytokines (e.g. IL-6) further enhance STAT1 activation and this was dis-inhibited in the absence of Fas. Thus, loss of Fas resulted in overwhelming STAT1 activation even under Th17 cell differentiation conditions which resulted in repression of pro-inflammatory Th17 cells.

The function of IFN-γ producing T cells in the CNS during EAE is complex and partly unresolved (Korn and Kallies, 2017). Mice deficient in IFN-γ, IFN-γ receptor, and STAT1 all develop more severe EAE (Bettelli et al., 2004; Tran et al., 2000), which was one of the observations that initially sparked the discovery of Th17 cells and argues for a protective role of Th1 cells in EAE. In contrast, the occurrence of IL-17A and IFN-γ double producing cells in the CNS correlates with disease severity in EAE, which are mostly derived from IL-17A single producing cells (Hirota et al., 2011). Induction of IFN-γ in Th17 cells is thought to be induced by activation of STAT4 by IL-23 in Th17 cells. The IL-17A and IFN-γ double positive cells are thought to be pathogenic although deletion of T-bet reduces their occurrence without considerably affecting EAE severity (Brucklacher-Waldert et al., 2016; Duhen et al., 2013). Based on the fate reporting experiments, global Fas-deficiency induces IFN-γ single positive cells before they can reach the Th17 lineage, while Th17-restricted Fas-deficiency causes Th17-to-Th1 instability. Both these processes appear to inhibit development of EAE. Of note, Fas-deficiency induces a greater extent of protection from EAE than deficiency of IL-17A or IL-17F (Haak et al., 2009; Komiyama et al., 2006). Indeed, Applicants previously identified a molecular signature that distinguishes pathogenic vs. non-pathogenic Th17 cells (Gaublomme et al., 2015; Lee et al., 2012). Preferential activation of STAT1 in Fas-deficient mice not only induces T-bet and a Th1 cell-like phenotype, but also interferes with the STAT3-mediated Th17 cell differentiation program thereby altering the phenotype and reducing the pathogenicity of Th17 cells in vivo.

Th17 cell development is critically dependent on STAT3 (Harris et al., 2007; Mathur et al., 2007) and humans with dominant negative mutations of STAT3 develop Hyper-immunoglobulin E syndrome characterized by impaired Th17 differentiation and greater susceptibility to fungal infections (Ma et al., 2008; Milner et al., 2008). STAT1 and STAT3 heterodimerize and increased STAT1 signalling will inhibit STAT3-dependent transcription driving Th17 cells (Villarino et al., 2017). Two recent studies demonstrate that STAT1 not only cross-inhibits STAT3-dependent transcription, but also dictates the specificity of the cytokines signalling through both STAT1 and STAT3 (Hirahara et al., 2015; Peters et al., 2015). Indeed, patients with STAT1 gain-of-function mutations show an impaired Th17 cell responses and develop chronic fungal infections (Liu et al., 2011). Thus, loss-of-function of STAT3 and gain-of-function of STAT1 both impair Th17 cell responses. Applicants find here that Fas was critical for sequestering STAT1 therefore tipping the balance in favour of STAT3's promotion of the Th17 cell differentiation program. However, loss of Fas released STAT1 and Applicants predict that this excessive availability of STAT1 in turn inhibits the Th17 cell gene program and promotes Th1 cell differentiation. The data also suggest that Fas controls the differentiation of other T helper cell lineages and may thus emerge as a regulator of multiple Th cell lineages through cross-inhibition of STAT proteins.

How can differential expression of Fas and FasL among Th lineages be reconciled with these findings? Fas expression in CD4⁺ T cells is driven by TCR engagement and is required for activation induced cell death (Krammer et al., 2007). Information on cytokine dependent regulation of Fas expression are limited. Consistent with the findings, previous studies have shown a greater susceptibility of Th1 cells to Fas-induced cell death compared to Th17 cells (Zhang et al., 2008b) and have attributed this to higher FasL expression in Th1 cells (Cencioni et al., 2015; Fang et al., 2010) or differential expression of anti-apoptotic proteins (Yu et al., 2009) rather than differential expression of Fas receptor. In contrast to FasL, previous studies did not report regulation of Fas expression by Th1 or Th17 cell inducing cytokines, but also did not perform mRNA expression profiling at high temporal resolution.

Applicants find Fas expression to be induced by activation alone and to be additionally regulated by cytokines. Applicants identified TGF-β1 to actively represses Fas expression which may explain why iTreg cell differentiation is unaffected by Fas-deficiency and Th17 cells generated with TGF-β1, IL-6 are less affected than Th17 cells differentiated with IL-1β, IL-6, IL-23. Consequently, Th1 cells are equipped with a greater density of ligand and receptor molecules than Th17 cells. However, FasL and Fas co-expressing cells do not die when sufficient survival signals are present and Fas receptor binding can have pro-proliferative effects under suboptimal TCR stimulation conditions (Kennedy et al., 1999; Paulsen et al., 2011). It may be under such conditions that Fas expression in Th cells limits commitment to the Th1 cell differentiation pathway by inhibiting STAT1 activation.

Several observations argue against a link between the defect of Th17 cell differentiation and lupus-like systemic autoimmunity in Fas-deficient mice. First, Fas^{-/-} mice were protected from EAE already at 6-10 weeks of age, which is before autoimmunity manifests on this genetic background (Cohen and Eisenberg, 1991). Second, T cell specific Fas deficiency in Cd4^{cre}Fas^{fl/fl} mice was sufficient to impair Th17 cell responses, without triggering autoimmunity (Hao et al., 2004). Third, limiting Fas deficiency to Th17 cells by utilizing an IL-17A^{Cre} mouse line affected their function in a cell intrinsic manner, but also did not induce autoimmunity (data not shown). Applicants thus demonstrate here that the ability of Fas to suppress lupus-like autoimmunity can be segregated from its Th17 cell-promoting function.

Reciprocal and antagonizing development is an essential and common theme in the directed differentiation of T helper cell lineages (Bettelli et al., 2006b; O'Shea and Paul, 2010). The previous transcriptional model predicted that positive regulators of Th17 cell differentiation would negatively impact on other T helper cell lineages and vice versa (Yosef et al., 2013). How can this be achieved? On a cell-extrinsic level, cytokines produced by one subset can cross-regulate differentiation of the other and evidence for this cell-extrinsic cross-regulation has been overwhelming (e.g. (Zhu et al., 2010)). However, in this report Applicants have provided evidence of how this is achieved in a cell-intrinsic manner: Fas was induced during Th17 cell differentiation, sequestered STAT1 and thereby limited Th1 cell differentiation and indirectly promoted Th17 differentiation.

In conclusion, Applicants here identified Fas as a cell intrinsic switch that not only regulates apoptosis, but also regulates competing differentiation pathways of T helper cell subsets in a cell intrinsic manner. The study also provides a link between Fas- and STAT1-signalling, and a mechanistic explanation of why Fas-deficient mice are resistant to EAE, yet are susceptible to systemic autoimmunity.

### Example 8—STAR Methods

### Experimental Animals

B6.Cg-Tg(Cd4-cre)1Cwi/BfluJ (named Cd4^{cre}) mice were purchased from Taconic. C57BL/6J, B6.SJL-Ptprc^{a}Pepc^{b}/BoyJ (named CD45.1), B6.PL-Thy1^{a}/CyJ (named CD90.1), B6.MRL-Tnfrsf6^{lpr}/J (i.e. 1pr mice on the C57BL/6 background; named Fas^{-/-} in the manuscript), B6Smn.C3-Fasl^{gld}/J (i.e. gld mice on the C57BL/6 background; named Fasl^{-/-}), B6N.129S1-Casp3^{tmlFlv}/J (named Casp3^{-/-}), B6.129S7-Ragl^{tmlMom}/J (named Ragl^{-/-}), B6; 129S6-Gt(ROSA)26Sor^{tm9(CAG-tdTomato)Hze}/J (named R26^{RFP}), B6.129S(Cg)-Stat1^{tmlDlv}/J (named Stat1^{-/-}) mice, and Il17a^{tm1.1(icre)Stck}/J (named Il17a^{cre}) were purchased from The Jackson Laboratories; C57BL/6-Fas^{tmlCgn}/J (named Fas^{fl/fl}) were described before (Hao et al., 2004) and were provided by Dr. Jin Wang, Baylor College of Medicine. C57BL/6-Tg(Tcra2D2,Tcrb2D2)1Kucha (named 2D2) mice have been previously described (Bettelli et al., 2006a)). Fas^{-/-} mice were crossed with 2D2 mice to generate Fas^{-/-}2D2 mice and with Stat1^{-/-} mice to generate Fas^{-/-}Stat1^{-/-} mice. CD45.1 mice were crossed with 2D2 mice to generate CD45.1-2D2 mice. C57BL/6J mice were used as wildtype controls for Fas^{-/-} and Casp3^{-/-} mice. For repletion experiments, 4*10⁶ MACS purified CD4⁺ T cells were intravenously injected into Ragl^{-/-} mice. 10-12 days after repletion, peripheral blood was analyzed for the presence of CD3⁺CD4⁺ cells and EAE was actively induced. Cre negative Fas^{fl/fl} littermates were used as controls for Cd4^{cre}Fas^{fl/fl} mice. Littermates heterozygous for both the Il17a^{cre} allele and for the Fas^{flox} allele were used as controls for the Il17a^{cre}Fas^{fl/fl} mice. Genotyping was done by routine PCR from tail biopsy derived DNA. All experiments were approved by and carried out in accordance with guidelines of the Institutional Animal Care and Use Committee (IACUC) at Harvard Medical School.

### Experimental Autoimmune Encephalomyelitis

Mice (6-10 weeks old) of both sexes at balanced ratio were immunized s.c. in the flanks with an emulsion containing the myelin oligodendrocyte glycoprotein (MOG) peptide MOG₃₅₋₅₅ (100 μg/mouse, Quality Controlled Biochemicals, >80% purity) and M. tuberculosis H37Ra extract (5 mg/ml, Difco Laboratories) in CFA (200 μl/mouse). Pertussis toxin (200 ng/mouse, List Biological Laboratories) was administered i.p. on days 0 and 2. Adoptive transfer EAE of Th17 cell differentiated 2D2 cells was performed as described (Jager et al., 2009). Briefly, CD4⁺CD44^{low}CD62L^{high}CD25⁻ naïve CD4⁺ T cells were purified by FACS sorting from 2D2 donor mice and cultured at 2*10⁶/ml in the presence of irradiated antigen presenting cells, soluble anti-CD3 antibody (2.5 μg/ml), IL-6 (30 ng/ml), TGF-β1 (3 ng/ml), anti-IFN-γ antibody, anti-IL-4 antibody (both at 20 μg/ml) for 2 days. Cells were subsequently split when necessary using IL-23 (10 ng/ml) containing media for 3 additional days and then plated at 2*10⁶/ml onto plates coated with anti-CD3/anti-CD28 (both at 2 μg/ml) in the absence of cytokines for 2 days. 5*10⁶ cytokine producing cells as assessed on day 2 after initial plating were intravenously injected into C57BL/6 recipients (6-10 weeks old). Animals were sex matched in transfer experiments. Mice were monitored and assigned grades for clinical signs of EAE using the following scoring system: 0, healthy; 1, limp tail; 2, impaired righting reflex or ataxic gait; 3, hind limb paralysis; 4, total limb paralysis; 5, moribund or death. Mice with a score of >4 were euthanized. If mice died during the course of the experiment, their clinical score of 5 was included in the analysis for the remainder of the experiment.

### Isolation of CNS-Infiltrating Mononuclear Cells

Mice were intracardially perfused with cold PBS. The forebrain and cerebellum were dissected and spinal cords flushed out from the spinal canal with PBS by hydrostatic pressure. CNS tissue was cut into pieces and digested with collagenase D (2.5 mg/ml, Roche Diagnostics) and DNase I (1 mg/ml, Sigma) at 37° C. for 20 min. Mononuclear cells were isolated by passing the tissue through a 70 μm cell strainer, followed by a 70%/37% percoll gradient centrifugation. Mononuclear cells were removed from the interphase, washed and re-suspended in culture medium for further analysis.

### CNS Histology

Histological analysis of CNS infiltration was performed as previously described (Jager et al., 2009). Briefly, mice were sacrificed 28 days after EAE induction or adoptive transfer of T effector cells and brains and spinal cords were fixed in 10% neutral-buffered formalin. Tissues were processed routinely for paraffin embedment and slides were stained with Luxol fast blue-H&E stains. Inflammatory foci (>10 mononuclear cells) were counted in leptomeninges and parenchyma in a blinded fashion.

### Enzyme-Linked Immunosorbent Assay

Secreted cytokines were measured after 96h of culture by sandwich enzyme-linked immunosorbent assay (ELISA) as previously described (Jager et al., 2009). The following antibody clones were used and purchased from Biolegend: IL-17A (TC11-18H10.1), IFN-γ (R4-6A2), IL-17A (TC11-8H4, biotinylated), IFN-γ (XMG1.2, biotinylated).

### Flow Cytometry and Related Reagents

For intracellular cytokine staining, cells were stimulated for 4 h at 37° C. with phorbol 12-myristate 13-acetate (PMA, 50 ng/ml; Sigma), ionomycin (1 μg/ml; Sigma) and monensin (GolgiStop; 1 μg/ml; BD Biosciences). After staining for surface markers, cells were fixed and permeabelized according to the manufacturer's instructions (BD Biosciences). FoxP3 staining was performed without PMA and ionomycin and GolgiStop stimulation using FoxP3 staining buffer (eBioscience). Staining phosphorylated STAT proteins for flow cytometry was performed by fixing first with 4% paraformaldehyde, second with 90% methanol at −20° C. and then using FoxP3 staining buffer as previously described (Peters et al., 2015). All flow cytometric data were collected on a FACS Calibur or FACS LSR II (both BD Biosciences) and analyzed using FloJo analysis software v7.6.5 (Tree Star, Inc.). The following antibodies (clone names in brackets) with different fluorochrome labels were purchased from Biolegend: anti-CD4 (RM4-5), anti-CD25 (PC61), anti-CD44 (IM7), anti-CD62L (Me114), anti-CD90.1 (OX-7), anti-IL-17A (TC11-18H10.1), anti-IFNγ (XMG1.2), anti-IL-5 (TRFK5), anti-CD45.1 (A20), anti-Vα3.2 (RR3-16). The following reagents were purchased from BD and used following the manufacturer's instructions: anti-STAT1 pY701 (4a), anti-STAT3 pY705 (4/P-STAT3), anti-Vβ11 (RR3-15), 7AAD, propidium iodide (PI), fluorochrome conjugated Annexin V, Annexin V staining buffer, intracellular cytokine staining kit, Apoptosis Detection Kit (APO-BRDU), Vybrant FAM caspase-8 assay kit. The following antibodies were purchased from eBioscience: anti-Foxp3 (FJK-16s), anti-IL-13 (eBio13A). Inhibitors for Caspase 1 (Z-YVAD-FMK), Caspase 3 (Z-DEVD-FMK), Caspase 8 (Z-IETD-FMK), and of Fas to Fas ligand interaction (Kp7-6) were purchased from EMD Millipore. Kp7-6 was dissolved in H₂O and used at 1 mM final concentration.

### Isolation and Differentiation of Naïve CD4⁺ T Cells.

CD4⁺CD44^{low}CD62L^{high}CD25⁻ naïve CD4⁺ T cells were purified by FACS sorting following MACS bead isolation of CD4⁺ cells. Naïve T cells were activated with plate-bound anti-CD3 (2 μg/ml, 145-2C11) and anti-CD28 (2 μg/ml, PV-1) antibody in 96-well (1×10⁵ cells/well) or 24-well flat bottom plates. All antibodies for cell culture were provided by the Brigham and Women's Hospital Antibody Core Facility. In vitro T cell differentiation was performed for 96 h. For non-pathogenic Th17 cell differentiation, cultures were supplemented with IL-6 (20 ng/ml) and TGF-β1 (2 ng/ml). For pathogenic Th17 cells differentiation cultures were supplemented with IL-1β (20 ng/ml), IL-6 (20 ng/ml) and IL-23 (10 ng/ml). Th1 cell differentiation was performed with IL-12 (20 ng/ml) and anti-IL-4 (11B11; 20m/m1). TNFα was used at 20 ng/ml. Differentiation of iTreg cells and Th2 cells were performed with TGF-β1 (5 ng/ml) and with IL-4 (20 ng/ml), anti-IL-12 (C17.8; 20 μg/ml), respectively. All cytokines were from R&D Systems.

### Immunofluorescence Microscopy

CD4⁺CD44^{low}CD62L^{high}CD25⁻ sorted naive CD4⁺ T cells were incubated on poly-L-lysine slides for 30 minutes in the presence of IL-6 (20 ng/ml), washed once with PBS, and fixed and permeabilized using Cytofix and Cytoperm (BD Biosciences) for 30 minutes. Samples were blocked with PBS/5% donkey serum (Abcam) overnight at 4° C. and subsequently stained with primary antibodies diluted in PBS/5% donkey serum for 1 hour, followed by three wash steps with PBS. The secondary antibody was diluted in PBS/5% donkey serum and incubated for 1 hour. After additional three wash steps, cells were mounted using DAPI containing mounting medium (CST) and visualized with a confocal laser scanning microscope LSM710 (Zeiss) within 24 hours. All incubations were performed at room temperature unless indicated otherwise. Antibodies were: rabbit anti-pSTAT1 S727 antibody (clone D3B7, CST, 1:300), FITC conjugated donkey anti-rabbit (Jackson Immuno, 1:1000), PE-conjugated mouse anti-pSTAT3 Y705 (clone 4/P-STAT3, BD, 1:300). Images were processed and quantified using ImageJ v1.47 (NIH). Five randomly chosen areas of each slide were photodocumented and 12 cells from each photo were randomly picked for analysis in a blinded fashion. For analysis of total protein content, randomly chosen cells were manually outlined and mean fluorescence was measured. For analysis of nuclear phospho-STAT, the nucleus was manually outlined in the DAPI and fluorescence overlay and nuclear fluorescence was quantified in the respective channel. Fluorescence intensity was quantified on a scale from zero (i.e. no intensity) to 255 (i.e. maximum intensity). All slides were processed in parallel and a minimum of 50 cells was analyzed per slide.

### Constructs and Luciferase Assays

The pCMV-Stat1 and pCMV-Stat3 vectors containing mouse cDNA sequences were obtained from the PlasmID Repository of Harvard Medical School. The pCMV-Fas construct was generated by PCR amplifying the Fas cDNA from a mouse cDNA library of differentiated Th17 cells from C57BL/6 mice and cloning the fragment in to an empty pCMV vector using EcoRI/NotI sites. The coding region was verified by Sanger sequencing. The pGL4-Il17a luciferase reporter construct containing 2kb of the mouse Il17a promoter region was provided by Warren Strober (Addgene plasmid #20124) (Zhang et al., 2008a). The Renilla construct was purchased from Promega.

EL4 cells (5×10⁴ cells/well, 96 well flat bottom plate) were cultured in the presence of soluble anti-CD3 and anti-CD28 (each 1 μg/ml) and transiently transfected with Renilla control and pGL4-Il17a luciferase vectors using Fugene (Roche). Expression vectors (pCMV-Stat1, pCMV-Stat3, pCMV-Fas) were co-transfected at a range of concentrations (0, 10, 20, 40 ng/ml). Total transfected DNA amount was maintained constant at 125 ng/well by adding empty pCMV vector to substitute for expression vectors. 48 hours after transfection, luminescence was measured with the Dual-Luciferase Reporter Assay System (Promega). The Firefly luciferase activity was normalized to Renilla luciferase activity. Data were normalized between experiments as fold-change versus empty vector control.

RNA Quantification Using nCounter and qPCR Analysis.

Gene expression was analysed using the nanostring method as previously described (Lee et al., 2015). Briefly, total RNA extracted using the RNeasy MicroKit (Quiagen) from >50,000 cells was hybridized for 16 hours at 65° C. together with the predefined capture and reporter codeset. The samples were then transferred onto the nanostring cartridge using the nCounter prep station. Cartridges were read at maximum resolution. A set of 250 predefined Th17 cell-related transcripts was used (Yosef et al., 2013). Transcripts were only considered if expression was >10 reads and differed >2.0-fold between experimental conditions. Data were calculated as fold regulation of wildtype. Visualization using heatmaps of nanostring data was performed using the GENE-E software. Gene expression was analysed using qPCR by extacting mRNA and reverse transcribing RNA into cDNA using the MultiMACS cDNA Synthesis Kit (Miltenyi) following the manufacturers instructions. cDNA served as template in TaqMan based qPCR analysis run on a ViiA7 (LifeTechnologies). TaqMan predesigned primer and probe mixes for the indicated transcripts were purchased from Applied Biosystems.

### RNA-Sequencing

For RNA-sequencing (RNA-seq), total RNA was purified from naïve T cells differentiated in the presence of either no cytokines or TGF-β1 (2 ng/ml) and IL-6 (20 ng/ml) for 48 hours using the RNeasy kit (Quiagen) including DNaseI treatment. Preparation of cDNA libraries for sequencing was performed using the RNA ligation method as previously described (Levin et al., 2010). Briefly, 1 μg of total RNA was polyA-selected twice using oligo-dT beads, then Zinc fragmented, DNase treated, and dephosphorylated. Then 3′ RNA-adapter was ligated to the dephosphorylated RNA using T4 RNA Ligase and first strand cDNA synthesis was performed using rTd primers and Affinity Script reverse transcriptase. RNA was then hydrolysed using NaOH. After clean-up, 5′ adapter was ligated to the cDNA and the cDNA was PCR amplified using adapter-complementary primers. Clean-up steps in between reactions were performed using Silane beads. Sequencing was performed on an Illumina HiSeq 2000.

### Transcriptional and Gene Set Enrichment Analysis

Differential expression analysis was performed with the Tuxedo Suite. Specifically, Applicants used TopHat (Trapnell et al., 2009) (version 2.0.10), running Bowtie (Langmead and Salzberg, 2012) (version 2.1.0.0) to generate alignments to the mouse genome assembly version 9 (mm9). These were used as input to the Cuffdiff (Trapnell et al., 2010) (version 2.1.1) program for differential expression computations. Applicants used Gene Set Enrichment Analysis (GSEA) to identify gene sets enriched in the RNA-seq data (Subramanian et al., 2005). Specifically, Applicants used the pre-ranked analysis mode, with differentially expressed RNA transcripts ranked by test statistics derived from Cuffdiff. The most significantly over-expressed genes were at the top of the ranked list, while the most under-expressed were at the bottom. Applicants used curated gene sets from pathway databases as available in the Molecular Signature Database (MSigDB) (Subramanian et al., 2005).

### Inferred Transcriptional Regulators

Applicants inferred transcriptional regulators responsible for the observed differential pattern of transcription using several approaches. First, Applicants gathered known mouse transcription factors (TF) from the MGI databases (Eppig et al., 2015) (www.informatics.jax.org) using the Gene Ontology term “DNA binding”. Then, in the direct approach, Applicants identified all differentially expressed (DE) TFs in the data using False Discovery Rate (FDR) assignment from Cuffdiff (Trapnell et al., 2010)_ENREF_51. However, TFs causal for differential expression do not have to be differentially expressed themselves at the time point of mRNA expression measurement. Therefore, Applicants searched for differentially expressed genes with binding motifs in their promoters that are associated with known TFs. This was accomplished by first downloading TRANSFAC (Matys et al., 2006)-based TF motifs and corresponding regulated gene lists from MSigDB. Next, using a Kolmogorov-Smirnov (KS) test Applicants assessed the enrichment of each gene list among expressed genes (FPKM>3) ordered by DE statistic scores. Applicants used a more stringent FDR algorithm (Benjamini and Yekutieli, 2001) for multiple hypothesis testing correction to better address non-independent tests due to overlapping gene lists. In addition, Applicants used the GSEA algorithm (pre-ranked mode) to identify candidate transcriptional regulators in the data using gene modules and their regulators derived from the Ontogenet model, based on ImmGen (www.immgen.org) consortium data.

### Protein-Protein Interaction (PPI) Data and Network Reconstruction

Applicants used a network analysis approach to predict which transcription factors were most likely affected by Fas given public protein-protein interaction databases and RNA-seq data generated in this study.

Applicants started with a background PPI network collected from the BioGRID (version 3.2.116), HPRD (release 9), and PhosphoSite (2014 Sep. 3) databases. Applicants selected data obtained from human and mouse systems. In BioGRID, Applicants used the following data types: Affinity Capture (except RNA), Biochemical Activity, Co-crystal structure, Co-localization, FRET, Far Western, PCA, Protein-peptide, Proximity Label-MS, Reconstituted Complex, Two-hybrid. From HPRD Applicants used in vivo and two-hybrid data. In this study, Applicants were interested in intracellular paths, therefore Applicants removed secreted proteins (according to UniProt annotations). Overall, Applicants obtained 107,573 interactions across 12,324 proteins.

Next, Applicants noted that network analysis cannot be accomplished trivially by looking up the transcription factors (DNA-binding proteins) in the network neighborhood of Fas, as there were only 4 of them that were directly connected (Casp8ap2, Trp63, Mbd4, Rara), while as many as 191 were connected by at most one intermediate node (FIG. 4e) and 766 by two nodes. Applicants therefore ranked the TFs based on the strength of network support for paths connecting them with Fas. Applicants scored those paths based on support by both expression data and better prior knowledge. To assign scores, Applicants noted that protein-protein interaction databases collect interactions that were observed in different cell types (and species), under differing experimental conditions, using various assays, etc. Therefore, a path connecting two proteins is just a possibility that does not have to hold in a specific system being studied. Applicants observe, however, that each additional path linking two proteins increases the chances that the proteins do influence each other. Applicants therefore computed and scored all paths (containing no more than two intermediate nodes) connecting Fas to any other TF in mouse genome. Each path score reflected the approximate belief that path proteins actually participated in carrying the signal between terminal nodes. Thus, the weight of an edge was set to:

Applicants=s*(1−(0.25)ⁿ),

where n is the number of publications supporting the interaction, 0.25 indicates assumed probability of the interaction being false despite the measurement (59,162 of interactions in the network were supported only by two-hybrid or affinity capture MS), and s is an estimate that the interaction takes place in the particular system assuming that interacting proteins are expressed.

Applicants set the node weight as a product of two components wₙ=wₙ₁ Wₙ₂. wₙ₁ reflected the belief that a given protein was actually expressed in the system and was set to:

wₙ₁=0.05+0.95(1−exp(−FPKM)),

where 0.05 indicates the assumed probability of a protein being expressed despite an observed RNA-seq FPKM equal 0, and the entire formula provides for a rapid increase with increasing FPKM. The second component, wₙ₂ reflected the belief that a protein was actively involved in expression changes due to Fas deficiency. For network nodes that were not known to be transcription factors Applicants used a uniform weight of 0.1. Applicants estimated the chances that a given TF was one of the causal ones for the observed differential expression phenotype in two ways: (1) using enrichments of known DNA-binding motifs, and (2) direct measurements of differential expression (described in “Inferred transcriptional regulators” above). Applicants used minimum FDR from two approaches in the weight computation

wn2=0.5(1−minFDR)+0.1 minFDR,

where 0.5 indicates the assumed probability that the TF node is active when assessed as differentially expressed or having a binding motif associated with DE genes, and 0.1 otherwise (the node weight for Fas was set to 1). Applicants did not distinguish between positively and negatively differentially expressed genes to allow for possible negative regulation. Applicants computed the network path score as a product of node and edge weights (this score ranges between 0 and 1 by definition). The sum of path scores between Fas and a TF reflected the evidence that Fas could influence it. Applicants avoided independently estimating s by separately estimating the influence evidence for paths of specific length. Stat1 had the highest influence score with network paths of having one intermediate node (FIG. 4e) and the second highest (after June) for paths with two intermediate nodes (Supplemental Table 1).

Applicants estimated the statistical significance of influence scores using randomized networks. Applicants shuffled edges between nodes, while preserving node degrees (Maslov and Sneppen, 2002) and also shuffled node weights. Applicants obtained one thousand randomized networks in this way, and in each of them computed influence scores. For each TF Applicants computed the corresponding empirical p-value (Supplemental Table 1).

### Co-Immunoprecipitation and Western Blotting

For DISC assembly, 1*10⁸ EL4 cells or 5-7*10⁷ MACS sorted primary mouse CD4 cells were stimulated for 15 minutes with either biotinylated anti-Fas antibody (Jo2, 2.5 μg/ml) and streptavidin (5m/ml, Thermo Scientific) or with multimerized recombinant human Fas ligand (aa 139-281) fused at the N-terminus to mouse ACRP30headless (aa 18-111) and a FLAG-tag (MegaFasL, Adipogen) at a final concentration of 100 ng/ml as indicated in the figure legends. MegaFasL was dissolved in H₂O that was used as vehicle. For immunoprecipitation (IP) experiments, cells were then washed twice with ice-cold PBS, lysed in ice-cold IP buffer containing 30 mM Tris HCL, 1% Triton-X100, 150 mM NaCl, 2 mM EDTA, 10% glycerol, 1 mM PMSF, phosphatase inhibitors (HALT, Thermo Scientific) and protease inhibitors (Roche). Immuoprecipitation was performed immediately after lysis using protein G magnetic Dynabeads (Life Technologies) with three consecutive washes with IP buffer. For phosphorylation studies, sorted naïve T cells were stimulated with the cytokines as indicated, then washed with PBS, and either lysed in IP buffer or cytoplasmic and nuclear fractions were extracted using NE-PER reagents (Thermo Scientific) following the manufacturer's protocol.

For Western blotting, proteins were denatured in the presence of β-mercaptoethanol, separated on pre-cast 4-12% Bis-Tris SDS-polyacrylamide gels and transferred onto PVDF membranes. Membranes were blocked with 5% non-fat dry milk (Bio-Rad) in TBS-T and primary and HRP coupled secondary antibodies were diluted at 1:1000 in 5% BSA in TBS-T. Detection was performed using ECL substrate (Thermo Scientific) and radiography films. Antibodies for Western blotting were purchased from Cell Signalling Technologies: rabbit anti-STAT1, rabbit anti-5727-phospho-STAT1 (D3B7), rabbit anti-Y701-phospho-STAT1 (58D6), rabbit anti-cFlip (D16A8), rabbit anti-Y705-phospho-STAT3 (D3A7), rabbit anti-STAT3 (D3Z2G), rabbit anti-STAT4 (C46B10), mouse anti-β-Actin (8H10D10), rabbit anti-GAPDH (D16H11), rabbit anti-TBP; or Santa Cruz: goat anti-FADD (M19), rabbit anti-FADD (H-181); or EMD Millipore: rat anti-Fas (7C10); or BD: hamster anti-Fas (Jo2). HRP conjugated secondary antibodies against heavy and light chain were: preadsorbed goat anti-rabbit IgG (Abcam), rabbit F(ab′)2 Anti-Goat IgG (Southern Biotech), horse anti-mouse IgG (Cell Signaling), goat anti-rat IgG (Santa Cruz).

### Statistics

The percentage of cytokine positive cells in in vitro differentiations was compared using Student's t-test for paired samples. All other results were analyzed by unpaired Student's t test. p<0.05 was considered significant. Statistical analysis was performed using GraphPad Prism 5.0.

### Supplemental Table 1

Panel ‘significant genes’ contains genes identified in the following tests: genes having significant RNA-seq differential expression (DE) with adjusted p-value (false discovery rate (FDR))<0.2. Panel ‘significant TF’: List of transcription factors (TF) with enriched binding motifs among Th17 DE genes; genes found in IFN-gamma pathway; transcription factors found as significant in one of the network analyses or genes found in ImmGen sets (‘fine183’ and ‘fine269’) or listed by ImmGen as regulators of these sets (‘fine183.reg’ and ‘fine269.reg’). Of note, Stat1 had a q-value of ˜0.16, and was observed with two other factors with similar statistical significance (q-value <0.2).

### REFERENCES

Afkarian, M., Sedy, J. R., Yang, J., Jacobson, N. G., Cereb, N., Yang, S. Y., Murphy, T. L., and Murphy, K. M. (2002). T-bet is a STAT1-induced regulator of IL-12R expression in naive CD4⁺ T cells. Nature immunology 3, 549-557.
Benjamini, Y., and Yekutieli, D. (2001). The control of the false discovery rate in multiple testing under dependency. Ann Stat 29, 1165-1188.
Bettelli, E., Baeten, D., Jager, A., Sobel, R. A., and Kuchroo, V. K. (2006a). Myelin oligodendrocyte glycoprotein-specific T and B cells cooperate to induce a Devic-like disease in mice. The Journal of clinical investigation 116, 2393-2402.
Bettelli, E., Carrier, Y., Gao, W., Korn, T., Strom, T. B., Oukka, M., Weiner, H. L., and Kuchroo, V. K. (2006b). Reciprocal developmental pathways for the generation of pathogenic effector TH17 and regulatory T cells. Nature 441, 235-238.
Bettelli, E., Pagany, M., Weiner, H. L., Linington, C., Sobel, R. A., and Kuchroo, V. K. (2003). Myelin oligodendrocyte glycoprotein-specific T cell receptor transgenic mice develop spontaneous autoimmune optic neuritis. The Journal of experimental medicine 197, 1073-1081.
Bettelli, E., Sullivan, B., Szabo, S. J., Sobel, R. A., Glimcher, L. H., and Kuchroo, V. K. (2004). Loss of T-bet, but not STAT1, prevents the development of experimental autoimmune encephalomyelitis. The Journal of experimental medicine 200, 79-87.
Brucklacher-Waldert, V., Ferreira, C., Innocentin, S., Kamdar, S., Withers, D. R., Kullberg, M. C., and Veldhoen, M. (2016). Tbet or Continued RORgammat Expression Is Not Required for Th17-Associated Immunopathology. J Immunol 196, 4893-4904.
Cencioni, M. T., Santini, S., Ruocco, G., Borsellino, G., De Bardi, M., Grasso, M. G., Ruggieri, S., Gasperini, C., Centonze, D., Barila, D., et al. (2015). FAS-ligand regulates differential activation-induced cell death of human T-helper 1 and 17 cells in healthy donors and multiple sclerosis patients. Cell death & disease 6, e1741.
Cohen, P. L., and Eisenberg, R. A. (1991). Lpr and gld: single gene models of systemic autoimmunity and lymphoproliferative disease. Annual review of immunology 9, 243-269.
Cua, D. J., Sherlock, J., Chen, Y., Murphy, C. A., Joyce, B., Seymour, B., Lucian, L., To, W., Kwan, S., Churakova, T., et al. (2003). Interleukin-23 rather than interleukin-12 is the critical cytokine for autoimmune inflammation of the brain. Nature 421, 744-748.
Duhen, R., Glatigny, S., Arbelaez, C. A., Blair, T. C., Oukka, M., and Bettelli, E. (2013). Cutting edge: the pathogenicity of IFN-gamma-producing Th17 cells is independent of T-bet. J Immunol 190, 4478-4482.
Eppig, J. T., Blake, J. A., Bult, C. J., Kadin, J. A., Richardson, J. E., and Mouse Genome Database, G. (2015). The Mouse Genome Database (MGD): facilitating mouse as a model for human biology and disease. Nucleic acids research 43, D726-736.
Fang, Y., Yu, S., Ellis, J. S., Sharav, T., and Braley-Mullen, H. (2010). Comparison of sensitivity of Th1, Th2, and Th17 cells to Fas-mediated apoptosis. Journal of leukocyte biology 87, 1019-1028.
Fortner, K. A., and Budd, R. C. (2005). The death receptor Fas (CD95/APO-1) mediates the deletion of T lymphocytes undergoing homeostatic proliferation. J Immunol 175, 4374-4382.
Gaublomme, J. T., Yosef, N., Lee, Y., Gertner, R. S., Yang, L. V., Wu, C., Pandolfi, P. P., Mak, T., Satija, R., Shalek, A. K., et al. (2015). Single-Cell Genomics Unveils Critical Regulators of Th17 Cell Pathogenicity. Cell.
Ghoreschi, K., Laurence, A., Yang, X. P., Tato, C. M., McGeachy, M. J., Konkel, J. E., Ramos, H. L., Wei, L., Davidson, T. S., Bouladoux, N., et al. (2010). Generation of pathogenic T(H)17 cells in the absence of TGF-beta signalling. Nature 467, 967-971.
Haak, S., Croxford, A. L., Kreymborg, K., Heppner, F. L., Pouly, S., Becher, B., and Waisman, A. (2009). IL-17A and IL-17F do not contribute vitally to autoimmune neuro-inflammation in mice. The Journal of clinical investigation 119, 61-69.
Hao, Z., Hampel, B., Yagita, H., and Rajewsky, K. (2004). T cell-specific ablation of Fas leads to Fas ligand-mediated lymphocyte depletion and inflammatory pulmonary fibrosis. The Journal of experimental medicine 199, 1355-1365.
Harris, T. J., Grosso, J. F., Yen, H. R., Xin, H., Kortylewski, M., Albesiano, E., Hipkiss, E. L., Getnet, D., Goldberg, M. V., Maris, C. H., et al. (2007). Cutting edge: An in vivo requirement for STAT3 signaling in TH17 development and TH17-dependent autoimmunity. J Immunol 179, 4313-4317.
Hirahara, K., Onodera, A., Villarino, A. V., Bonelli, M., Sciume, G., Laurence, A., Sun, H. W., Brooks, S. R., Vahedi, G., Shih, H. Y., et al. (2015). Asymmetric Action of STAT Transcription Factors Drives Transcriptional Outputs and Cytokine Specificity. Immunity 42, 877-889.
Hirota, K., Duarte, J. H., Veldhoen, M., Hornsby, E., Li, Y., Cua, D. J., Ahlfors, H., Wilhelm, C., Tolaini, M., Menzel, U., et al. (2011). Fate mapping of IL-17-producing T cells in inflammatory responses. Nature immunology 12, 255-263.
Holzelova, E., Vonarbourg, C., Stolzenberg, M. C., Arkwright, P. D., Selz, F., Prieur, A. M., Blanche, S., Bartunkova, J., Vilmer, E., Fischer, A., et al. (2004). Autoimmune lymphoproliferative syndrome with somatic Fas mutations. The New England journal of medicine 351, 1409-1418.
Hu, X., and Ivashkiv, L. B. (2009). Cross-regulation of signaling pathways by interferon-gamma: implications for immune responses and autoimmune diseases. Immunity 31, 539-550.
Jager, A., Dardalhon, V., Sobel, R. A., Bettelli, E., and Kuchroo, V. K. (2009). Th1, Th17, and Th9 effector cells induce experimental autoimmune encephalomyelitis with different pathological phenotypes. J Immunol 183, 7169-7177.
Kano, S., Sato, K., Morishita, Y., Vollstedt, S., Kim, S., Bishop, K., Honda, K., Kubo, M., and Taniguchi, T. (2008). The contribution of transcription factor IRF1 to the interferon-gamma-interleukin 12 signaling axis and TH1 versus TH-17 differentiation of CD4⁺ T cells. Nature immunology 9, 34-41.
Kennedy, N. J., Kataoka, T., Tschopp, J., and Budd, R. C. (1999). Caspase activation is required for T cell proliferation. The Journal of experimental medicine 190, 1891-1896.
Komiyama, Y., Nakae, S., Matsuki, T., Nambu, A., Ishigame, H., Kakuta, S., Sudo, K., and Iwakura, Y. (2006). IL-17 plays an important role in the development of experimental autoimmune encephalomyelitis. J Immunol 177, 566-573.
Korn, T., Bettelli, E., Oukka, M., and Kuchroo, V. K. (2009). IL-17 and Th17 Cells. Annual review of immunology 27, 485-517.
Korn, T., and Kallies, A. (2017). T cell responses in the central nervous system. Nature reviews. Immunology 17, 179-194.
Krammer, P. H., Arnold, R., and Lavrik, I. N. (2007). Life and death in peripheral T cells. Nature reviews. Immunology 7, 532-542.
Langmead, B., and Salzberg, S. L. (2012). Fast gapped-read alignment with Bowtie 2. Nature methods 9, 357-359.
Lee, Y., Awasthi, A., Yosef, N., Quintana, F. J., Xiao, S., Peters, A., Wu, C., Kleinewietfeld, M., Kunder, S., Hafler, D. A., et al. (2012). Induction and molecular signature of pathogenic TH17 cells. Nature immunology 13, 991-999.
Lee, Y., Mitsdoerffer, M., Xiao, S., Gu, G., Sobel, R. A., and Kuchroo, V. K. (2015). IL-21R signaling is critical for induction of spontaneous experimental autoimmune encephalomyelitis. The Journal of clinical investigation.
Levin, J. Z., Yassour, M., Adiconis, X., Nusbaum, C., Thompson, D. A., Friedman, N., Gnirke, A., and Regev, A. (2010). Comprehensive comparative analysis of strand-specific RNA sequencing methods. Nature methods 7, 709-715.
Liu, L., Okada, S., Kong, X. F., Kreins, A. Y., Cypowyj, S., Abhyankar, A., Toubiana, J., Itan, Y., Audry, M., Nitschke, P., et al. (2011). Gain-of-function human STAT1 mutations impair IL-17 immunity and underlie chronic mucocutaneous candidiasis. The Journal of experimental medicine 208, 1635-1648.
Ma, C. S., Chew, G. Y., Simpson, N., Priyadarshi, A., Wong, M., Grimbacher, B., Fulcher, D. A., Tangye, S. G., and Cook, M. C. (2008). Deficiency of Th17 cells in hyper IgE syndrome due to mutations in STAT3. The Journal of experimental medicine 205, 1551-1557.
Maslov, S., and Sneppen, K. (2002). Specificity and stability in topology of protein networks. Science 296, 910-913.
Mathur, A. N., Chang, H. C., Zisoulis, D. G., Stritesky, G. L., Yu, Q., O'Malley, J. T., Kapur, R., Levy, D. E., Kansas, G. S., and Kaplan, M. H. (2007). Stat3 and Stat4 direct development of IL-17-secreting Th cells. J Immunol 178, 4901-4907.
Matys, V., Kel-Margoulis, O. V., Fricke, E., Liebich, I., Land, S., Barre-Dirrie, A., Reuter, I., Chekmenev, D., Krull, M., Hornischer, K., et al. (2006). TRANSFAC and its module TRANSCompel: transcriptional gene regulation in eukaryotes. Nucleic acids research 34, D108-110.
Meyer Zu Horste, G., Wu, C., Wang, C., Cong, L., Pawlak, M., Lee, Y., Elyaman, W., Xiao, S., Regev, A., and Kuchroo, V. K. (2016). RBPJ Controls Development of Pathogenic Th17 Cells by Regulating IL-23 Receptor Expression. Cell reports 16, 392-404.
Milner, J. D., Brenchley, J. M., Laurence, A., Freeman, A. F., Hill, B. J., Elias, K. M., Kanno, Y., Spalding, C., Elloumi, H. Z., Paulson, M. L., et al. (2008). Impaired T(H)17 cell differentiation in subjects with autosomal dominant hyper-IgE syndrome. Nature 452, 773-776.
O'Shea, J. J., and Paul, W. E. (2010). Mechanisms underlying lineage commitment and plasticity of helper CD4+ T cells. Science 327, 1098-1102.
Paulsen, M., Valentin, S., Mathew, B., Adam-Klages, S., Bertsch, U., Lavrik, I., Krammer, P. H., Kabelitz, D., and Janssen, 0. (2011). Modulation of CD4⁺ T-cell activation by CD95 co-stimulation. Cell death and differentiation 18, 619-631.
Peters, A., Fowler, K. D., Chalmin, F., Merkler, D., Kuchroo, V. K., and Pot, C. (2015). IL-27 Induces Th17 Differentiation in the Absence of STAT1 Signaling. J Immunol 195, 4144-4153.
Sabelko, K. A., Kelly, K. A., Nahm, M. H., Cross, A. H., and Russell, J. H. (1997). Fas and Fas ligand enhance the pathogenesis of experimental allergic encephalomyelitis, but are not essential for immune privilege in the central nervous system. J Immunol 159, 3096-3099.
Strasser, A., Jost, P. J., and Nagata, S. (2009). The many roles of FAS receptor signaling in the immune system. Immunity 30, 180-192.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proceedings of the National Academy of Sciences of the United States of America 102, 15545-15550.
Takahashi, T., Tanaka, M., Brannan, C. I., Jenkins, N. A., Copeland, N. G., Suda, T., and Nagata, S. (1994). Generalized lymphoproliferative disease in mice, caused by a point mutation in the Fas ligand. Cell 76, 969-976.
Tran, E. H., Prince, E. N., and Owens, T. (2000). IFN-gamma shapes immune invasion of the central nervous system via regulation of chemokines. J Immunol 164, 2759-2768.
Trapnell, C., Pachter, L., and Salzberg, S. L. (2009). TopHat: discovering splice junctions with RNA-Seq. Bioinformatics 25, 1105-1111.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nature biotechnology 28, 511-515.
Villarino, A. V., Kanno, Y., and O'Shea, J. J. (2017). Mechanisms and consequences of Jak-STAT signaling in the immune system. Nature immunology 18, 374-384.
Waldner, H., Collins, M., and Kuchroo, V. K. (2004). Activation of antigen-presenting cells by microbial products breaks self tolerance and induces autoimmune disease. The Journal of clinical investigation 113, 990-997.
Waldner, H., Sobel, R. A., Howard, E., and Kuchroo, V. K. (1997). Fas- and FasL-deficient mice are resistant to induction of autoimmune encephalomyelitis. J Immunol 159, 3100-3103.
Walsh, C. M., Wen, B. G., Chinnaiyan, A. M., O'Rourke, K., Dixit, V. M., and Hedrick, S. M. (1998). A role for FADD in T cell activation and development. Immunity 8, 439-449.
Wang, Y., Wu, T. R., Cai, S., Welte, T., and Chin, Y. E. (2000). Stat1 as a component of tumor necrosis factor alpha receptor 1-TRADD signaling complex to inhibit NF-kappaB activation. Molecular and cellular biology 20, 4505-4512.
Watanabe-Fukunaga, R., Brannan, C. I., Copeland, N. G., Jenkins, N. A., and Nagata, S. (1992). Lymphoproliferation disorder in mice explained by defects in Fas antigen that mediates apoptosis. Nature 356, 314-317.
Xiao, S., Yosef, N., Yang, J., Wang, Y., Zhou, L., Zhu, C., Wu, C., Baloglu, E., Schmidt, D., Ramesh, R., et al. (2014). Small-molecule RORgammat antagonists inhibit T helper 17 cell transcriptional network by divergent mechanisms. Immunity 40, 477-489.
Yang, X. O., Panopoulos, A. D., Nurieva, R., Chang, S. H., Wang, D., Watowich, S. S., and Dong, C. (2007). STAT3 regulates cytokine-mediated generation of inflammatory helper T cells. The Journal of biological chemistry 282, 9358-9363.
Yosef, N., Shalek, A. K., Gaublomme, J. T., Jin, H., Lee, Y., Awasthi, A., Wu, C., Karwacz, K., Xiao, S., Jorgolli, M., et al. (2013). Dynamic regulatory network controlling TH17 cell differentiation. Nature 496, 461-468.
Yu, Y., Iclozan, C., Yamazaki, T., Yang, X., Anasetti, C., Dong, C., and Yu, X. Z. (2009). Abundant c-Fas-associated death domain-like interleukin-1-converting enzyme inhibitory protein expression determines resistance of T helper 17 cells to activation-induced cell death. Blood 114, 1026-1028.
Zhang, F., Meng, G., and Strober, W. (2008a). Interactions among the transcription factors Runxl, RORgammat and Foxp3 regulate the differentiation of interleukin 17-producing T cells. Nature immunology 9, 1297-1306.
Zhang, Y., Xu, G., Zhang, L., Roberts, A. I., and Shi, Y. (2008b). Th17 cells undergo Fas-mediated activation-induced cell death independent of IFN-gamma. J Immunol 181, 190-196.
Zheng, Y., Ouaaz, F., Bruzzo, P., Singh, V., Gerondakis, S., and Beg, A. A. (2001). NF-kappa B RelA (p 65) is essential for TNF-alpha-induced fas expression but dispensable for both TCR-induced expression and activation-induced cell death. J Immunol 166, 4949-4957.
Zhu, J., Yamane, H., and Paul, W. E. (2010). Differentiation of effector CD4 T cell populations (*). Annual review of immunology 28, 445-489.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An isolated T cell modified to comprise altered FAS-STAT1 binding, preferably, wherein the T cell is a Th17 cell or naïve Th0 cell.

2. The isolated T cell of claim 1,
wherein the T cell is modified to express a recombinant polypeptide capable of antagonizing FAS-STAT1 interaction, preferably, wherein the polypeptide does not affect the binding of FAS to FAS-L and/or wherein the polypeptide does not affect the binding of FAS to FADD; or
wherein the T cell is modified to express a recombinant polypeptide that is capable of adopting a FAS ligand bound conformation, is inactivated for apoptotic signaling, and is able to bind to STAT1, preferably, wherein the polypeptide does not affect the binding of FAS to FAS-L and/or wherein the polypeptide does not affect the binding of FAS to FADD; or
wherein the T cell is modified to over-express STAT1; or
wherein the T cell is modified to abolish or knockdown expression or activity of STAT1 and is differentiated under Th17 conditions, preferably, wherein Th17 conditions comprise cultures supplemented with IL-6 and TGF-β1 or supplemented with IL-1β, IL-6 and IL-23; or
wherein the T cell comprises a genetic modifying agent targeting STAT1; or
wherein the T cell is modified to comprise a non-silent mutation in FAS and/or STAT1, wherein the mutation inhibits FAS-STAT1 binding, preferably, wherein the T cell comprises a genetic modifying agent targeting FAS and/or STAT1; or
wherein the T cell is modified to decrease, but not eliminate expression or activity of FAS, preferably, wherein the T cell is differentiated under Th17 conditions, more preferably, wherein Th17 conditions comprise cultures supplemented with IL-6 and TGF-β1 or supplemented with IL-1β, IL-6 and IL-23; or
wherein the T cell comprises a genetic modifying agent targeting FAS.

3-11. (canceled)

12. The isolated T cell of claim 2, wherein the genetic modifying agent comprises a CRISPR system, a zinc finger nuclease system, a TALEN, or a meganuclease, preferably,
wherein the CRISPR system comprises Cas9 or Cpf1 and targets the STAT1 gene; or
wherein the CRISPR system comprises a Cas13 system and targets STAT1 mRNA, more preferably, wherein the Cas13 system comprises Cas13-ADAR; or
wherein the CRISPR system comprises a Cas13 system and targets FAS and/or STAT1 mRNA, preferably, wherein the Cas13 system comprises Cas13-ADAR; or
wherein the CRISPR system comprises a Cas13 system and targets FAS mRNA, preferably, wherein the Cas13 system comprises Cas13-ADAR.

13-16. (canceled)

17. The isolated T cell of claim 2, wherein the non-silent mutation alters a post-translational modification site in FAS and/or STAT1 that alters FAS-STAT1 binding, preferably, wherein the mutation does not inhibit FAS apoptotic signaling.

18-31. (canceled)

32. The isolated T cell of claim 1, wherein the T cell is a tumor infiltrating lymphocyte (TIL); and/or
wherein the T cell expresses an endogenous T cell receptor (TCR) or chimeric antigen receptor (CAR) specific for a tumor antigen; and/or
wherein the T cell is expanded; and/or
wherein the T cell is modified to express a suicide gene, wherein the modified T cell can be eliminated upon administration of a drug.

33-35. (canceled)

36. A pharmaceutical composition comprising the isolated T cell of claim 1.

37-41. (canceled)

42. A method of treating cancer comprising administering the pharmaceutical composition of claim 36 to a subject in need thereof,
wherein a Th17 response is enhanced; or
wherein a Th1 response is enhanced.

43. (canceled)

44. A method of treating an inflammatory or autoimmune disease comprising administering the pharmaceutical composition of claim 36 to a subject in need thereof, wherein a Th17 response is reduced.

45. A method of modulating T cell balance, the method comprising perturbing FAS-STAT1 binding in a T cell or a population of T cells,
preferably, wherein the T cell or population of T cells comprise naïve Th0 T cells; and
wherein the cells are cultured under Th1 or Th17 conditions, preferably, wherein Th17 conditions comprise cultures supplemented with IL-6 and TGF-β1 or supplemented with IL-1β, IL-6 and IL-23.

46. The method of claim 45, wherein perturbing comprises introducing a genetic modifying agent targeting FAS and/or STAT1 to the T cell or population of T cells, preferably, wherein the genetic modifying agent comprises a CRISPR system, a zinc finger nuclease system, a TALEN, or a meganuclease, preferably, wherein the CRISPR system comprises a Cas13 system and targets FAS and/or STAT1 mRNA, more preferably, wherein the Cas13 system comprises Cas13-ADAR; or
wherein the method comprises contacting the T cell or population of T cells with an inhibitor of FAS-STAT1 binding; or
wherein the method comprises increasing expression of STAT1 in the T cell or population of T cells; or
wherein the method comprises providing the T cell or population of T cells with a FAS polypeptide, wherein said polypeptide is able to bind to STAT1, preferably, wherein the polypeptide adopts a FAS ligand bound conformation and is inactivated for apoptotic signaling.

47-49. (canceled)

50. The method of claim 46, wherein the T cell or population of T cells is modified to comprise a non-silent mutation in FAS and/or STAT1, wherein the mutation inhibits FAS-STAT1 binding, preferably, wherein the mutation alters a post-translational modification site in FAS and/or STAT1; or
wherein the T cell or population of T cells is modified to comprise a decrease or knockout in expression of STAT1; or
wherein the T cell or population of T cells is modified to comprise a decrease in expression of FAS, preferably, wherein FAS mRNA is targeted and the decrease is temporary.

51. (canceled)

52. The method of claim 45, wherein T cell differentiation is shifted towards Th1 cells and/or is shifted away from Th17 cells; or
wherein T cell differentiation is shifted towards Th17 cells and/or is shifted away from Th1 cells.

53-57. (canceled)

58. The method of claim 46, wherein the CRISPR system is administered as a ribonucleoprotein (RNP) complex.

59-64. (canceled)

65. The method of claim 45, wherein FAS is bound by FAS ligand.

66. A method of modulating an immune response in a subject in need thereof, the method comprising:
administering to the subject a therapeutically effective amount of an inhibitor of FAS-STAT1 binding; or
administering Th17 cells to the subject, wherein the Th17 cells are modified to comprise altered FAS-STAT1 binding.

67. The method of claim 66, wherein the method is for treating an aberrant immune response in said subject, preferably,
wherein the method is for treating an autoimmune disease, preferably, wherein the autoimmune disease is selected from Multiple Sclerosis (MS), Irritable Bowel Disease (IBD), Crohn's disease, spondyloarthritides, Systemic Lupus Erythematosus (SLE), Vitiligo, rheumatoid arthritis, psoriasis, Sjögren's syndrome, and diabetes; or
wherein the method is for treating an inflammatory disorder, preferably, wherein the inflammatory disorder is selected from psoriasis, inflammatory bowel diseases (IBD), allergic asthma, food allergies and rheumatoid arthritis.

68-71. (canceled)

72. The method of claim 66, wherein the inhibitor of FAS-STAT1 binding does not affect the binding of FAS to FAS-L; and/or
wherein the inhibitor of FAS-STAT1 binding does not affect the binding of FAS to FADD; and/or
wherein the inhibitor binds to the cytoplasmic domain of FAS; and/or
wherein the inhibitor does not bind to the extracellular domain of FAS.

73-75. (canceled)

76. The method of claim 66, wherein the inhibitor is an antibody, antibody fragment, intrabody, antibody-like protein scaffold, polypeptide, genetic modifying agent, or small molecule, preferably, wherein the genetic modifying agent comprises a CRISPR system, a zinc finger nuclease system, a TALEN, or a meganuclease.

77. (canceled)

78. A pharmaceutical composition comprising an inhibitor of FAS-STAT1 binding, preferably,
wherein the inhibitor of FAS-STAT1 binding does not affect the binding of FAS to FAS-L; and/or
wherein the inhibitor of FAS-STAT1 binding does not affect the binding of FAS to FADD; and/or
wherein the inhibitor binds to the cytoplasmic domain of FAS; and/or
wherein the inhibitor does not bind to the extracellular domain of FAS; and/or
wherein the inhibitor is an antibody, antibody fragment, intrabody, antibody-like protein scaffold, polypeptide, genetic modifying agent, or small molecule.

79-86. (canceled)

87. The method of claim 46, wherein providing a FAS polypeptide comprises providing a nucleic acid encoding the polypeptide, preferably, wherein said nucleic acid is provided as a vector.

88. (canceled)

89. The method of claim 46, wherein the polypeptide is a membrane bound polypeptide, preferably,
wherein the polypeptide does not bind to FAS-L; and/or
wherein the polypeptide does not comprise the extracellular domain of FAS; and/or
wherein binding of the polypeptide to STAT1 does not lead to phosphorylation of STAT1; and/or
wherein binding of the polypeptide to STAT1 prevents or reduces nuclear translocation of STAT1.

90-93. (canceled)

94. The method of claim 66, wherein the method is for treating cancer or an infectious disease in a subject in need thereof, the method comprising:
(a) isolating Th17 cells from the blood of the subject;
(b) transforming the isolated Th17 cells with one or more vectors encoding:
(i) a CAR or endogenous TCR directed against a tumor antigen or an infectious disease antigen, and
(ii) a CRISPR system targeting STAT1; and
(c) administering the transformed Th17 cells to the subject.

95. The method of claim 66, wherein the method is for treating autoimmunity in a subject in need thereof, the method comprising:
a) isolating Th17 cells from the blood of the subject;
b) transforming the isolated Th17 cells with one or more vectors encoding a CRISPR system targeting FAS; and
c) administering the Th17 FAS mutant cells to the subject.

96. A method of screening for agents capable of modulating FAS-STAT1 interaction comprising:
a) differentiating naïve Th0 T cells under Th17 conditions in the presence of one or more agents that specifically bind to FAS and/or STAT1, preferably, wherein the cells are differentiated under pathogenic Th17 conditions; and
b) detecting one or more Th17 or Th1 markers, wherein decreased Th17 markers or increased Th1 markers indicates an agent that modulates the interaction, preferably, wherein the one or more Th17 markers comprises IL-17A.

97-98. (canceled)


SEQUENCE LISTING (6 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Artificial Sequence
  Synthetic Peptide
IEVMYPPPY

SEQ ID NO: 3 - protein, 107 aa, Homo sapiens; identical to SEQ ID NO: 1

SEQ ID NO: 4 - protein, 356 aa, Homo sapiens
MGSGGGGSEGAPKKKRKVGSSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 5 - protein, 255 aa, Artificial Sequence
  Synthetic Peptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQL

SEQ ID NO: 6 - protein, 183 aa, Artificial Sequence
  Synthetic Peptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS